SEQUENCE LISTING (24 sequences)

SEQ ID NO: 1 - protein, 9 aa, Homo sapiens
LLGRNSFEV

SEQ ID NO: 2 - protein, 9 aa, Homo sapiens
STPPPGTRV

SEQ ID NO: 3 - protein, 6 aa, Artificial Sequence
  Description of Artificial Sequence Synthetic 6xHis tag
HHHHHH

SEQ ID NO: 4 - protein, 10 aa, Artificial Sequence
  Description of Artificial Sequence Synthetic peptide
ASGGGGSGGG

SEQ ID NO: 5 - protein, 20 aa, Artificial Sequence
  Description of Artificial Sequence Synthetic peptide
GGGGSGGGGSGGGGSGGGGS

SEQ ID NO: 6 - protein, 9 aa, Artificial Sequence
  Description of Artificial Sequence Synthetic peptide
EEEEYMPME

SEQ ID NO: 7 - DNA, 33 bp, Artificial Sequence
  Description of Artificial Sequence Synthetic oligonucleotide
tttcgtacgtcttgtcccagtcagtgacgcagc

SEQ ID NO: 8 - protein, 20 aa, Artificial Sequence
  Description of Artificial Sequence Synthetic peptide
METDTLLLWVLLLWVPGSTG

SEQ ID NO: 9 - protein, 113 aa, Mus sp.
QSVTQPDARVTVSEGASLQLRCKYSYSGTPYLFWYVQYPRQGLQLLLKYYSGDPVVQGVNGFEAEFSKSNSSFHLRKASVHWSDSAVYFCVLSEDSNYQLIWGSGTKLIIKPD

SEQ ID NO: 10 - protein, 24 aa, Artificial Sequence
  Description of Artificial Sequence Synthetic peptide
TSGGGGSGGGGSGGGGSGGGGSSS

SEQ ID NO: 11 - protein, 114 aa, Homo sapiens
NSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLSGGGTEVFFGKGTRLTVV

SEQ ID NO: 12 - protein, 130 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 13 - protein, 18 aa, Artificial Sequence
  Description of Artificial Sequence Synthetic peptide
VNAKTTAPSVYPLAPVSG

SEQ ID NO: 14 - protein, 133 aa, Homo sapiens [sequence omitted]

SEQ ID NO: 15 - protein, 552 aa, Artificial Sequence
  Description of Artificial Sequence Synthetic polypeptide
METDTLLLWVLLLWVPGSTGQSVTQPDARVTVSEGASLQLRCKYSYSGTPYLFWYVQYPRQGLQLLLKYYSGDPVVQGVNGFEAEFSKSNSSFHLRKASVHWSDSAVYFCVLSEDSNYQLIWGSGTKLIIKPDTSGGGGSGGGGSGGGGSGGGGSSSNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLSGGGTEVFFGKGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADVNAKTTAPSVYPLAPVSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 16 - protein, 532 aa, Artificial Sequence
  Description of Artificial Sequence Synthetic polypeptide
QSVTQPDARVTVSEGASLQLRCKYSYSGTPYLFWYVQYPRQGLQLLLKYYSGDPVVQGVNGFEAEFSKSNSSFHLRKASVHWSDSAVYFCVLSEDSNYQLIWGSGTKLIIKPDTSGGGGSGGGGSGGGGSGGGGSSSNSKVIQTPRYLVKGQGQKAKMRCIPEKGHPVVFWYQQNKNNEFKFLINFQNQEVLQQIDMTEKRFSAECPSNSPCSLEIQSSEAGDSALYLCASSLSGGGTEVFFGKGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADVNAKTTAPSVYPLAPVSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 17 - DNA, 60 bp, Artificial Sequence
  Description of Artificial Sequence Synthetic oligonucleotide
atggagacagacacactcctgttatgggtactgctgctctgggttccaggttccaccggt

SEQ ID NO: 18 - DNA, 339 bp, Mus sp.
cagtcagtgacgcagcccgatgctcgcgtcactgtctctgaaggagcctctctgcagctgagatgcaagtattcctactctgggacaccttatctgttctggtatgtccagtacccgcggcaggggctgcagctgctcctcaagtactattcaggagacccagtggttcaaggagtgaatggcttcgaggctgagttcagcaagagtaactcttccttccacctgcggaaagcctctgtgcactggagcgactctgctgtgtacttctgtgttttgagcgaggatagcaactatcagttgatctggggctctgggaccaagctaattataaagccagac

SEQ ID NO: 19 - DNA, 72 bp, Artificial Sequence
  Description of Artificial Sequence Synthetic oligonucleotide
actagtggtggcggtggcagcggcggtggtggttccggtggcggcggttctggcggtggcggttcctcgagc

SEQ ID NO: 20 - DNA, 342 bp, Homo sapiens
aattcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgtcagggggcggcacagaagttttctttggtaaaggaaccagactcacagttgta

SEQ ID NO: 21 - DNA, 390 bp, Homo sapiens
gaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagac

SEQ ID NO: 22 - DNA, 54 bp, Artificial Sequence
  Description of Artificial Sequence Synthetic oligonucleotide
gttaacgcaaagacaaccgccccttcagtatatccactagcgcccgtttccgga

SEQ ID NO: 23 - DNA, 402 bp, Homo sapiens
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaacttaa

SEQ ID NO: 24 - DNA, 1659 bp, Artificial Sequence
  Description of Artificial Sequence Synthetic polynucleotide
atggagacagacacactcctgttatgggtactgctgctctgggttccaggttccaccggtcagtcagtgacgcagcccgatgctcgcgtcactgtctctgaaggagcctctctgcagctgagatgcaagtattcctactctgggacaccttatctgttctggtatgtccagtacccgcggcaggggctgcagctgctcctcaagtactattcaggagacccagtggttcaaggagtgaatggcttcgaggctgagttcagcaagagtaactcttccttccacctgcggaaagcctctgtgcactggagcgactctgctgtgtacttctgtgttttgagcgaggatagcaactatcagttgatctggggctctgggaccaagctaattataaagccagacactagtggtggcggtggcagcggcggtggtggttccggtggcggcggttctggcggtggcggttcctcgagcaattcaaaagtcattcagactccaagatatctggtgaaagggcaaggacaaaaagcaaagatgaggtgtatccctgaaaagggacatccagttgtattctggtatcaacaaaataagaacaatgagtttaaatttttgattaactttcagaatcaagaagttcttcagcaaatagacatgactgaaaaacgattctctgctgagtgtccttcaaactcaccttgcagcctagaaattcagtcctctgaggcaggagactcagcactgtacctctgtgccagcagtctgtcagggggcggcacagaagttttctttggtaaaggaaccagactcacagttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagacgttaacgcaaagacaaccgccccttcagtatatccactagcgcccgtttccggagcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactaacttaa

(19) United States
(12) Patent Application Publication
(10) Pub. No.: US 2020/0283498 A1
(43) Pub. Date: Sep. 10, 2020

(54) T CELL RECEPTOR FUSIONS AND CONJUGATES AND METHODS OF USE THEREOF

(71) Applicant: Altor Bioscience LLC, Miramar, FL (US)

(72) Inventors: Hing C. Wong, Weston, FL (US); Peter Rhode, Miami, FL (US); Xiaoyun Zhu, Weston, FL (US)

(21) Appl. No.: 16/835,267

(22) Filed: Mar. 30, 2020

Related U.S. Application Data
(63) Continuation of application No. 16/530,748, filed on Aug. 2, 2019.
Application No. 16/530,748 is a continuation of application No. 16/508,279, filed on Jul. 10, 2019.
Application No. 16/508,279 is a continuation of application No. 14/303,026, filed on Jun. 12, 2014.
Application No. 14/303,026 is a continuation of application No. 12/933,644, filed on Dec. 6, 2010, now abandoned.
(60) Provisional application No. 61/070,100, filed on Mar. 19, 2008.

(51) Int. Cl. C07K 14/725; C07K 14/535; C07K 14/55; C07K 14/565; C07K 14/56; C07K 16/46

(52) U.S. Cl. CPC C07K 14/7051; C07K 14/535; C07K 14/55; A61K 38/00; C07K 14/56; C07K 16/46; C07K 14/565

(57) ABSTRACT

Featured is T cell receptor complexes designed to redirect the immune system against various diseases. The T cell receptor complexes of the invention have been engineered to recognize target antigen in a functionally bispecific nature. Fusion protein complexes and protein conjugate complexes are comprised of high affinity antigen-specific TCR and biologically active proteins and/or effector molecules. Also featured is methods of production of T cell receptor fusion and conjugate complexes as well as therapeutic compositions for use of the complexes.

### RELATED APPLICATIONS

This application is a Continuation of application Ser. No. 16/530,748 filed on Aug. 20, 2019; which is a Continuation of application Ser. No. 16/508,279 filed on Jul. 10, 2019. Application Ser. No. 16/508,279 is a Continuation of application Ser. No. 14/303,026 filed on Jun. 12, 2014. Application Ser. No. 14/303,026 is a Continuation of application Ser. No. 12/933,644 filed on Dec. 6, 2010. Application Ser. No. 12/933,644 is a national stage application filed under 35 U.S.C. § 371 of International Application PCT/US2009/001728 filed Mar. 19, 2009. Application PCT/US2009/001728 claims the benefit of U.S. Provisional Application 61/070,100 filed on Mar. 19, 2008. The entire contents of these applications are incorporated herein by reference in their entirety.

### INCORPORATION BY REFERENCE OF SEQUENCE LISTING

The contents of the sequence listing text file named “048277-519C04US_Sequence_Listing.txt”, which was created on Sep. 20, 2010 and is 22,164 bytes in size, are incorporated herein by reference in its entirety.

### FIELD OF INVENTION

The present invention relates to soluble T cell receptor complexes and more particularly to soluble T cell receptor fusion complexes and soluble T cell receptor conjugate complexes, as well as methods for making and using such molecules. The provided molecules are useful for a variety of therapeutic applications as well as diagnostic purposes.

### BACKGROUND OF THE INVENTION

Traditional approaches to the treatment of diseases such as cancers, autoimmune, and infective (including viral, bacterial, parasitic and fungal) diseases, have included surgery, radiation chemotherapy, antibiotics or combination therapies. However, such therapies have not proven effective against a majority of these indications. Development of alternate remedies for preventing and/or treating human diseases is crucial. In recent years immunotherapy and gene therapy approaches utilizing antibodies and T-lymphocytes have emerged as new and promising methods for treating human disease.

One such approach to treatment has included use of antibodies for targeting of therapeutic or diagnostic agents to particular targets. Numerous groups have made developments revolving around the use of antibodies as a targeting agent. Such developments have included construction of antibody fusion proteins and antibody conjugate molecules linking antibodies to various effector molecules, including radioactive molecules, chemotherapeutics agents, toxins, and additional bioactive proteins. Therapeutics or diagnostics developed using such molecules are designed to cause a particular effect which is targeted by the linked antibody.

Just as antibodies have been developed as therapeutics, additional primary effectors of the immune system, T cell receptors (TCR), have unique advantages as a platform for developing therapeutics. While antibodies are limited to recognition of pathogens in the blood and extracellular spaces or to protein targets on the cell surface, T cell receptors can recognize antigens displayed with MHC molecules on the surfaces of cells (including antigens derived from intracellular proteins). Depending on the subtype of T cells that recognize displayed antigen and become activated, T cell receptors and T cells harboring T cell receptors can participate in controlling various immune responses. For instance, T cells are involved in regulation of the humoral immune response through induction of differentiation of B cells into antibody producing cells. In addition, activated T cells act to initiate cell-mediated immune responses. Thus, T cell receptors can recognize additional targets not available to antibodies.

A T-cell response is modulated by antigen binding to a T-cell receptor (TCR). One type of TCR is a membrane bound heterodimer consisting of an α and β chain resembling an immunoglobin variable (V) and constant (C) region. The TCR α chain includes a covalently linked V-α and C-α chain, whereas the chain includes a V-β chain covalently linked to a C-β chain. The V-α and V-β chains form a pocket or cleft that can bind a superantigen or antigen in the context of a major histocompatibility complex (MHC) (known in humans as an HLA complex). See generally Davis Ann. Rev. of Immunology 3: 537 (1985); Fundamental Immunology 3rd Ed., W. Paul Ed. Rsen Press LTD. New York (1993).

The TCR is believed to play an important role in the development and function of the immune system. For example, the TCR has been reported to mediate cell killing, increase B cell proliferation, and impact the development and severity of various disorders including cancer, allergies, viral infections and autoimmune disorders.

Therapeutic use of cytokines if often limited by the short half life of the cytokine once administered to a subject. For example, the rhIL-2 has a very short half life, and the therapeutic use of IL-2 is limited. Patients receiving rhIL-2 drug need to be infused every 8 hours during the FDA-approved 5 day treatment cycle.

It thus would be desirable to provide novel targeting agents based on T cell receptors, as well as methods for producing and using such agents for therapeutic and diagnostic settings. It would be particularly desirable to provide such molecules that would have certain advantages in comparison to prior art complexes based on antibody targeting and that had improved pharmacokinetic properties that allowed for more favorable administration.

### SUMMARY OF THE INVENTION

We have now created a class of modified TCR complexes that have improved therapeutic utility. These modified TCRs have increased serum half life and/or increased cell surface residency time. Due to these properties, the complexes, e.g., fusion molecules, exhibit improved therapeutic activity. The modified TCRs can be used to guide, target or direct localized toxic agents to specific sites to intervene in a disease process. For example, a TCR, which specifically recognizes a peptide derived from a cancer associated protein that is displayed by an MHC molecule, can be fused or conjugated to a biologically active molecule and thereby guide that molecule to the cancer cell to effect a desirable therapeutic outcome.

The TCRs of the invention can be modified in ways that link the TCR to the biologically active molecule. This invention teaches the use of genetic fusions and chemical conjugation as methods for effecting such linkage. The TCR to which the biologically active molecule can be attached is a native TCR heterodimer or soluble versions thereof, or more preferably soluble, single-chain TCR. The biologically active molecules can be a variety of bioactive effector molecules, polypeptides and non-protein molecules including, but not limited to, cytokines, chemokines, growth factors, protein or non-protein toxins, immunoglobulin domains, cytotoxic agents, chemotherapeutic agents, radioactive materials, detectable labels, and the like.

In some instances, the soluble sc-TCR proteins will include one or more fused effectors or tags. For example, in some cases the tags can be used to help purify the TCR protein fusion complex from naturally-occurring cell components which typically accompany the fusion protein. In other cases, the protein tag can be used to introduce a pre-determined chemical or proteolytic cleavage site into the soluble protein. Particularly, contemplated is introduction of a segment encoding a tag into a DNA vector, e.g., between sequence encoding the fusion complex and the effector molecule chain or suitable fragment so that the TCR molecule can be cleaved (i.e. separated) from the effector chain or fragment if desired.

Particularly preferred T cell receptor molecules for use in the invention are single chain T cell receptors.

In a preferred aspect of the invention, a TCR fusion complex is covalently linked to an immunoglobulin such as IgG, IgM, or IgA or fragment thereof (e.g., Fab, Fab′, F(ab′)₂). Suitably the TCR fusion complex is linked to constant regions of the immunoglobulin.

In another preferred aspect of the invention, a TCR fusion complex is covalently linked to a cytokine, such as IL-2 for example.

Yet another preferred aspect of the invention includes, a TCR fusion complex is covalently linked to a chemokines, such as MIP-1β for example.

Further, another preferred aspect of the invention, a TCR fusion complex is covalently linked to a growth factors, such as GM-CSF or G-CSF for example.

In another preferred aspect of the invention, a TCR fusion complex is covalently linked to a protein or non-protein toxin, such as ricin for example.

Further, in another preferred aspect of the invention, a TCR fusion complex is covalently linked to a cytotoxic agent, such as doxorubicin for example.

In another preferred aspect of the invention, a TCR fusion complex is covalently linked to a radioactive materials, such as I¹²⁵ for example.

Still another preferred aspect of the invention includes, a TCR fusion complex is covalently linked to a detectable labels, such as fluorescent, radioactive or electron transfer agents, for example.

Specifically provided are soluble TCR fusion proteins and TCR conjugate complexes that include an effector that is a cell toxin or a detectably-labelled atom or compound suitable for diagnostic, imaging, or therapeutic studies. The TCR fusion complexes and TCR conjugate complexes can be used in a variety of applications including detection and/or imaging cells or tissue in vivo, as well as therapeutic uses such as damaging or killing cells in vitro or in vivo. In general, targeted cells or tissue will include one or more ligands capable of selectively binding the TCR. Exemplary cells include tumor cells such as melanoma and virally-infected cells (e.g., cells infected with a primate DNA or RNA virus such as cytomegalovirus or the AIDS virus, respectively).

Other aspects and embodiments of the invention are discussed below.

### BRIEF DESCRIPTION OF THE DRAWINGS

FIG. 1A, FIG. 1B, and FIG. 1C are a construct design and expression of soluble 264 single-chain (sc) T cell receptor-kappa constant chain fusion protein (TCR-κ).
FIG. 1A is a schematic representing the 264 TCR constructed as a three-domain scTCR covalently linked to the kappa constant chain region.
FIG. 1B is a coomassie blue stain of a protein gel containing purified 264 scTCR-κ fusion protein run under reduced and non-reduced conditions.
FIG. 1C is an immuno-blot of purified 264 scTCR-κ fusion protein probed with an anti-kappa-horseradish peroxidase (HRP)-labeled conjugate.

FIG. 2A, FIG. 2B, and FIG. 2C represent the construct design and expression of a soluble 264 scTCR-IL-2 fusion protein.
FIG. 2A is a schematic showing the 264 scTCR gene covalently linked to the IL-2 gene with the EE peptide tag included to facilitate detection of the molecule.
FIG. 2B is a coomassie blue stain of a protein gel containing purified 264 scTCR-IL-2 and 264 scTCR-κ fusion proteins.
FIG. 2C is an immunoblot analysis of the purified 264 scTCR-IL-2 fusion protein probed with an anti-EE tag mAb and a goat anti-mouse-HRP conjugate.

FIG. 3 is demonstrative results of IL-2 activity in a bioassay.

FIG. 4A and FIG. 4B are demonstrative results of an antigen presenting cell stained with the 264 scTCR-IL2 fusion protein.

FIG. 5A, FIG. 5B, FIG. 5C, and FIG. 5D are demonstrative results of a cell conjugation assay.

FIG. 6 is a schematic for formats for T cell receptor based therapeutic agents.

FIG. 7 is a schematic of tumor cell killing mediated by scTCR targeted drug delivery.

FIG. 8 is a schematic of tumor cell killing mediated by Fc dependent cell-mediated cytotoxicity.

FIG. 9 is a schematic illustration of the pNAG2 vector.

FIG. 10 is a schematic drawing showing the pSUN27 vector.

FIG. 11 is a drawing showing preferred bispecific hybrid molecules pBISP/D011.10 and pBISP/149.

FIG. 12A is a schematic drawing showing a method for making a chimeric bispecific antibody molecule. The method uses a hybridoma-expressing cell (145-2C11 hybridoma) to produce antibody chains (heavy lines) that combine with an sc-TCR/Ig fusion molecule (light chain) inside the cell. FIG. 12B is a schematic illustrating a preferred structure for the sc-TCR/Ig molecule.

FIG. 13 is a schematic drawing showing the vector pSUN7 vector.

FIG. 14A and FIG. 14B depict MHC/peptide binding ability of the TCR portion of 264scTCR/IL-2 fusion protein. T2 cells were loaded with p53 (aa 264-272) peptide (green line) or p53 (aa 149-157) peptide (red line), and stained with either 264scTCR/IL-2 fusion protein and anti-TCR Cβ mAb as shown in FIG. 14rA or 264scTCR/IL-2 fusion protein and anti-IL-2 mAb as shown in FIG. 14B.

FIG. 15A and FIG. 15B depict IL-2 receptor binding ability of the IL-2 portion of 264scTCR/IL-2 fusion protein. FIG. 15A is a graph wherein stimulated mouse splenocytes were labeled with 264scTCR/IL-2 fusion protein and stained with HLA-A2 p53 (aa 264-272) tetramers (green line). The control is splenocytes stained with HLA-A2 p53 (aa 264-272) tetramers only (red line). FIG. 15B graph wherein CTLL-2 mouse cytotoxic T lymphocytes were labeled with 264scTCR/IL-2 fusion protein and stained with HLA-A2 p53 (264-272) tetramers (green line). The control is CTLL-2 cells stained with a 264scTCR/kappa fusion protein (red line).

FIG. 16 depicts cell surface residency time of TCR/IL-2 fusions. Cell surface binding of rhIL-2, 264scTCR/IL-2 and MART-1scTCR/IL-2 to IL-2R+ CTLL-2 cells was assessed by flow cytometry. Data points represent normalized level of protein remaining bound to CTLL-2 surface. Curves represent fit of data to a one-phase exponential decay model. Representative results of four independent experiments.

FIG. 17 depicts the in vitro immune cell proliferative activity of scTCR/IL-2 fusion proteins. CTLL-2 cells were subjected to WST-1 proliferation assays after 2 days of culture with 264scTCR/IL-2, MART-1scTCR/IL-2 or rhIL-2. Each curve represents normalized proliferative responses. Data points are means±SE of triplicate determinations. EC50 and 95% CI interval were estimated by four-parameter logistic curve fitting analysis.

FIG. 18A and FIG. 18B depict an in vitro immune stimulatory activity of scTCR/IL-2 fusion proteins. FIG. 18A is a graph showing the lytic activity of scTCR/IL-2 fusion protein-activated nude mouse splenocytes was compared to unstimulated (control) or rhIL-2-activated splenocytes in calcein release assays using YAC-1 target cells at different E:T ratios. The specific cytotoxicity was calculated using the following formula: percentage of cytotoxicity=(fluorescent intensity (FI) of cell supernatant of test sample−FI of target cells with medium)÷(FI of target cells treated with 0.04% Triton X-100−FI of target cells with medium)×100. Representative results of two independent experiments. FIG. 18B is a depiction of the lytic activity of human PBMCs incubated for 11 hours with 260 IU/mL of 264scTCR/IL-2 or rhIL-2 was compared to unstimulated human PBMCs in calcein release assays using K562 target cells at different E:T ratios.

FIG. 19A and FIG. 19B depict in vivo activation of immune cells in mice following scTCR/IL-2 treatment. Activation of splenocytes was compared following repeated administration of 264scTCR/IL-2 or MART-1scTCR/IL-2 in nude mice bearing A375 xenografts. A375 tumor-bearing nude mice injected i.v. with 264scTCR/IL-2 or MART-1scTCR/IL-2 at 0, 24, 48, and 72 h. Splenocytes were isolated at the indicated times and analyzed by flow cytometry for expression of CD25, CD122 or pan-NK markers. The plots indicate the percentage of CD25, CD122 or pan-NK-positive staining (dark line) compared with splenocyte staining with an isotype control mAb (red line).

FIG. 20A, FIG. 20B, FIG. 20C, and FIG. 20D depict in vitro conjugation of IL-2 receptor-bearing immune cells with antigen presenting target cells mediated by scTCR/IL-2 fusion protein. T2 cells were loaded with either p53 (aa 264-272) or p53 (aa 149-157) (control) peptides and then labeled with HE. CTLL-2 cells were labeled with calcein AM. Labeled cells were mixed and incubated in the presence or absence of 264scTCR/IL-2 fusion protein and the samples were analyzed by flow cytometry. Assay conditions including loading peptide used and presence or absence of fusion protein are indicated beneath each histogram. Single stained regions are marked 1 and 3 and the double stained cell poplulation is marked 2.

FIG. 21A and FIG. 21B are line graphs showing a comparison of the pharmacokinetic profiles of scTCR/IL-2 fusion proteins and rhIL-2. FIG. 21A is a line graph wherein 264scTCR/IL-2 or rhIL-2 were administered intravenously to HLA-A2.1/K^{b}-transgenic mice and serum levels of the proteins were measured by ELISA. The anti-IL2 Ab ELISA measures the concentration of intact 264scTCR/IL-2 molecule whereas the W4F Ab ELISA measures serum concentration of the TCR Cβ domain. The observed concentrations are represented by symbols and the model-fitted curves are represented by lines. FIG. 21B is a line graph wherein comparative pharmacokinetics of MART-1scTCR/IL-2, 264scTCR/IL-2 and rhIL-2 proteins administered intravenously in HLA-A2.1/K^{b}-transgenic mice.

FIG. 22A and FIG. 22B depict the effect of scTCR/IL-2 on growth of primary tumors in nude mice. A375 tumor cells (1×10⁶ cells) were injected s.c. into nude mice on study day 1 and tumors were allowed to establish for three days (FIG. 22A), allowed to grow to 200 mm3 (FIG. 22B). Mice were then treated i.v. with 264scTCR/IL-2 (1.6 mg/kg/dose) or rhIL-2 (0.4 mg/kg/dose) daily for 4 days, followed by treatment every other day for a total of 9 doses. Tumors were measured at least twice a week and tumor volumes (mean±SD) were plotted.

FIG. 23A and FIG. 23B depict the dose dependent effect of scTCR/IL-2 fusion protein on growth of primary tumors in nude mice. FIG. 23A is a line graph wherein subcutaneous A375 tumors were established in nude mice as described. Mice were treated with molar equivalents of IL-2, 264scTCR/IL-2, or PBS for 4 days, followed by treatment every other day for a total of 9 doses. Tumors were measured twice a week. FIG. 23B is a line graph wherein tumor weights following study completion plotted for each tumor for each treatment group. Bars represent SD; *, P<0.05 compared with IL-2 (0.4 mg/kg).

FIG. 24A and FIG. 24B depict the effect of scTCR/IL-2 on growth of primary tumors in nude mice. FIG. 24A is a line graph wherein subcutaneous A375 tumor bearing nude mice were treated i.v. with rhIL-2, 264scTCR/IL-2, or the dose volume equivalent of PBS for four daily injections, followed by a 10-day rest period and then four more daily injections. FIG. 24B is a line graph wherein subcutaneous A375 tumor bearing nude mice were treated with 0.5 mg/kg 264scTCR/IL-2 (dosing indicated by solid arrows) or with a 3.2-fold higher molar equivalent amount per dose (0.4 mg/kg) of rhIL-2 every 8 hrs for five days followed by a 10-day rest period and then every 8 hrs for five more daily injections (dosing indicated by dashed arrows). Tumor volumes were measured at least twice a week and were plotted as mean±SEM.

FIG. 25A, FIG. 25B, FIG. 25C, and FIG. 25D depict the effects of scTCR/IL-2 fusion proteins on growth of primary p53+ HLA-A2+ tumors in nude mice. FIG. 25A is a line graph wherein subcutaneous A375 tumor bearing nude mice were treated i.v. with either 0.5 mg/kg 264scTCR/IL-2 or control TCR/IL-2 fusion (MART1scTCR/IL-2) or a molar equivalent of rhIL-2 with four daily injections, followed by a 10-day rest period and then four more daily injections. FIG. 25B-FIG. 25D is a series of line graphs wherein nude mice were injected s.c. with p53+/HLA-A2.1+ MDA-MB-231 tumor cells (FIG. 25B), p53+/HLA-A2.1+ PANC1 tumor cells (FIG. 25C), or p53−/HLA-A2.1− AsPC1 tumor cells (FIG. 25D) and tumors were allowed to establish to ˜25-100 mm³. Mice were then randomized (5-8 mice per group) and injected i.v. with molar equivalents of rhIL-2, 264scTCR/IL-2, or the dose equivalent volume of PBS for 4 days, followed by every other day for a total of 9 doses. Tumor volumes were measured at least twice a week and are plotted as mean±SEM.

FIG. 26 demonstrates that scTCR/IL-2 effects are mediated via cells of the innate immune system. Tumors from nude mice treated with 264scTCR/IL-2 or rhIL-2 were fixed in zinc fixative, embedded in paraffin, sectioned, and stained with an anti-CD45R antibody detecting NK or B cells and photographed at ×200. Representative staining of A375 (left) and AsPC1 (right) tumor sections are shown for each treatment as indicated. Distinct populations of CD45R+ cells are observed within the tumor proper of A375 tumors treated with 264scTCR/IL-2 (brown stained cells) (upper left). However, in IL-2 treated A375 tumors (lower left), as well as 264scTCR/IL-2 and IL-2 treated AsPC1 tumors (upper and lower right), these cells remained in the mouse stroma.

FIG. 27 depicts scTCR/IL-2 treatment-dependent infiltration of immune effector cells into tumors in nude mice. CD45R+ NK cells stained by immunohistochemistry were quantified in A375 tumors obtained from nude mice 24 h after last of four daily treatments with 264scTCR/IL-2, MART-1scTCR/IL-2 or PBS. The density of CD45R+NK cells per field of view were tabulated and plotted. Boxes, interquartile range of distribution (25th-75th percentile); line with in box, median; whisker, 5^{th}-95^{th} percentile. *, p=0.028; **, p=0.028. At least three tumors per group and 10 fields per tumor were analyzed.

FIG. 28 depicts targeted tumor infiltration of adoptively transferred splenocytes pre-coated with scTCR/IL-2. Quantitation of CD45R+ cell density (left) and human TCR-coated cells (right) in A375 tumors obtained from nude mice 24 hours after adoptive transfer of 264scTCR/IL-2 or MART-1scTCR/IL-2 treated splenocytes. Boxes, interquartile range of distribution (25^{th}-75^{th} percentile); line within box, median; whisker, 5^{th}-9 percentile. *, P<0.0001; **, P<0.0001. At least three tumors per group and 10 fields per tumor were analyzed.

FIG. 29 depicts the effects of in vitro incubation of NK cells with scTCR/IL2 fusion on the survival of xenograft tumor-bearing nude mice. Athymic nude mice were injected with human NSCLC A549-A2 cells to allow establishment of lung metastases. Purified NK cells isolated from spleens of allogenic donor mice were incubated in vitro with rhIL-2, MART1scTCR-IL2, c264scTCR-IL2 or c264scTCR-IL15/c264scTCR-IL15Rα and adoptively transferred into the tumor-bearing mice that had been pretreated with cyclophosphamide (CTX), as indicated in the figure legend. The percent survival following treatment was plotted.

FIG. 30 depicts MHC/peptide binding ability of the TCR portion of 264scTCR/GM-CSF fusion protein. T2 cells were loaded with p53 (aa 264-272) peptide (pink line), and stained with 264scTCR/GM-CSF fusion protein and anti-GM-CSF mAb. Other lines represent 264scTCR/GM-CSF staining of T2 cells loaded with p53 (aa 149-157) peptide.

FIG. 31 depicts the in vitro immune cell proliferative activity of scTCR/GM-CSF fusion proteins. TF-1 cells were subjected to WST-1 proliferation assays after culture with equivalent amounts of 264scTCR/GM-CSF or GM-CSF, as indicated in the figure legend. Each curve represents the level of cell proliferation as measured by absorbance of WST-1 product comparing the amounts of added fusion protein or GM-CSF.

FIG. 32 depicts the in vitro inhibition of cancer cell proliferation by scTCR/IFN-α fusion proteins. 5T33 or 5T33GP33 myeloma cells were subjected to WST-1 proliferation assays after culture with equivalent amounts of LCMVscTCR/IFNα or IFNα. Each curve represents the level of cell proliferation as measured by absorbance of WST-1 product comparing the amounts of added fusion protein or IFNα.

FIG. 33A is a line graph showing serum IFN-γ levels (determined by ELISA analysis) of subjects following treatment with 0.04 mg/kg ALT-801 (MTD level). FIG. 33B is a line graph showing IFN-γ ELISPOT analysis of PBMCs for subjects treated with 0.04 mg/kg ALT-801 (MTD level). Peripheral blood mononuclear cells (PBMCs) were prepared from samples collected pre-dosing (Cycles 1 & 2) and 2 hours after the drug infusion of dose 3 and 7. Following overnight incubation in medium alone or medium containing ALT-801, p53-positive A375 tumor cells or ALT-801+A375 tumor cells, activated immune effector cells were measured using ELISPOT for IFN-γ production

FIG. 34 sets forth Table 1 depicting a comparison of common Adverse Events reported for high-dose rhIL-2 treatment with those observed for ALT-801 administered at the MTD dose level (0.04 mg/kg).

FIG. 35 sets forth Table 2 depicting preliminary tumor assessment for evaluable subjects treated with 0.015, 0.04 or 0.08 mg/kg ALT-801. PD, progressive disease (>+25% change in sum of longest diameter (LD) of target lesions compared to baseline measurement); SD, stable disease (≤+25% and >−30% change in sum of longest diameter (LD) of target lesions compared to baseline measurement). For SD (highlighted), change in target lesions compared to baseline is indicated at week 7 or 11 after the first dose.

FIG. 36A and FIG. 36B set forth the amino acid sequence of ALT-801 (SEQ ID NOS 8-16, respectively, in order of appearance).

FIG. 37A and FIG. 37B sets forth the nucleic acid sequence of ALT-801 (SEQ ID NOS 17-24, respectively, in order of appearance).

### DETAILED DESCRIPTION OF THE INVENTION

In an attempt to improve upon the performance of antibody-based molecules, we have developed a class of antigen-specific therapeutics based on use of T cell receptors (TCRs). Previous work demonstrates that TCR-based reagents have several advantages over antibody molecules. First, antibody-based therapies are often associated with lower than expected killing efficiency of tumor cells due to shedding of tumor antigens. Although there are reports of MHC shedding, the levels of specific MHC/tumor peptide in circulation are much lower than free circulating tumor antigen. Second, antibody molecules fail to recognize many potential tumor antigens because they are not exposed on the surface of the cells or not accessible to the antibody molecule. Many potential tumor specific proteins are intracellular but are normally processed within the cell into peptides which are then presented in the context of either MHC class I or MHC class II molecules on the surface of the tumor cell. Unlike TCRs, antibodies do not generally recognize these processed antigens occupying the binding clefts of MHC molecules. Third, many of the antigens recognized by antibodies are heterogeneic by nature, which limits the effectiveness of an antibody to a single tumor histology. In contrast, many T cell epitopes are common to a broad range of tumors originating from several distinct tissues.

As summarized above, we have now created a class of TCR fusion and conjugate complexes that have significantly increased utility as therapeutic molecules. Specifically, the new class of fusion molecules has been created that has increased cell surface residency time, and improved pharmacokinetic profiles, e.g., these molecules have a longer plasma half life. In certain embodiments, the molecules have half lives that are 5, 10, 15, 20, 25, 30, 35, 40, 45, 50, 100 or more times longer than the components of the fusion molecules alone. The invention also provides expression vectors that encode such complexes, that comprise a TCR molecule covalently linked to a biologically active polypeptide or molecule, and methods for production and use of such fusion and conjugate complexes and expression vectors and conjugate complexes.

A T cell recognizes antigen presented on the surfaces of cells by means of the T cell receptors expressed on their cell surface. TCRs are disulfide linked heterodimers, most consisting of α and β chain glycoproteins. T cells use mechanisms to generate diversity in their receptor molecules similar to those mechanisms for generating antibody diversity operating in B cells (Janeway and Travers; Immunobiology 1997). Similar to the immunoglobulin genes, TCR genes are composed of segments that rearrange during development of T cells. TCR polypeptides consist of amino terminal variable and carboxy terminal constant regions. While the carboxy terminal region functions as a transmembrane anchor and participates in intracellular signaling when the receptor is occupied, the variable region is responsible for recognition of antigens. The TCR α chain contains variable regions encoded by V and D segments only, while the chain contains additional joining (J) segments. The rearrangement of these segments and the mutation and maturation of the variable regions results in a diverse repertoire of TCRs capable of recognizing an incredibly large number of different antigens displayed in the context of different TCR molecules.

Technology has been developed previously to produce highly specific T cell receptors (TCR) which recognize particular antigen. For example, the pending U.S. patent application Ser. No. 08/813,781 and U.S. Ser. No. 09/422,375, incorporated herein by reference; and International publications PCT/US98/04274 and PCT/US99/24645, and references discussed therein disclose methods of preparing and using specific TCRs. Additionally, particular specific TCRs have been produced by recombinant methods as soluble, single-chain TCRs (scTCR). Methods for production and use of scTCRs have been disclosed and are described in pending U.S. patent application Ser. No. 08/943,086, and International application PCT/US98/20263 which are incorporated herein by reference. Such TCRs and scTCRs can be altered so as to create fusions or conjugates to render the resulting TCRs and scTCRs useful as therapeutics. The TCR complexes of the invention can be generated by genetically fusing the recombinantly produced TCR or scTCR coding region to genes encoding biologically active polypeptide or molecules to produce TCR fusion complexes. Alternatively, a TCR or scTCRs can also be chemically conjugated with biologically active molecules to produce TCR conjugate complexes.

By the term “fusion molecule” as it is used herein is meant a TCR molecule and an effector molecule usually a protein or peptide sequence covalently linked (i.e. fused) by recombinant, chemical or other suitable method. If desired, the fusion molecule can be fused at one or several sites through a peptide linker sequence. Alternatively, the peptide linker may be used to assist in construction of the fusion molecule. Specifically preferred fusion molecules are fusion proteins. The fusion molecules of the invention exhibit improved characteristics that make them better therapeutic molecules.

The term “increased cell surface residency time” as used herein is meant to indicate tha the claimed fusion molecules associate with proteins on the surface of cell for a longer period of time than any component of the fusion molecule does alone. In certain embodiments, the cell surface residency time is increased by 20%, 30%, 40%, 50%, 60%, 70%, 80%, 90%, 100% or more.

The term “serum half life” or “plasma half life” as used herein is intended to indicate the amount of time that is required for the concentration or amount of fusion molecule of the invention when in the body to be reduced to exactly one-half of a given concentration or amount. The fusion molecules of the invention display significantly longer half lives than the biological effector molecule when not in a fusion molecule. For example, the serum half life of the disclosed molecules can increase by 20%, 30%, 40%, 50%, 60%, 70%, 80%, 90%, 100%, 200%, 300%, 400%, 500%, 750%, 1000%, 1250%, 1500%, 1750%, 2000% or more over the serum half life of the components of the claimed molecules when not part of a fusion protein.

A “polypeptide” refers to any polymer preferably consisting essentially of any of the 20 natural amino acids regardless of its size. Although the term “protein” is often used in reference to relatively large proteins, and “peptide” is often used in reference to small polypeptides, use of these terms in the field often overlaps. The term “polypeptide” refers generally to proteins, polypeptides, and peptides unless otherwise noted. Peptides useful in accordance with the present invention in general will be generally between about 0.1 to 100 KD or greater up to about 1000 KD, preferably between about 0.1, 0.2, 0.5, 1, 2, 5, 10, 20, 30 and 50 KD as judged by standard molecule sizing techniques such as centrifugation or SDS-polyacrylamide gel electropheresis.

By the term “conjugate molecule” as it is used herein is meant a TCR molecule and an effector molecule usually a chemical or synthesized molecule covalently linked (i.e. fused) by chemical or other suitable method. If desired, the conjugate molecule can be fused at one or several sites through a peptide linker sequence or a carrier molecule. Alternatively, the peptide linker or carrier may be used to assist in construction of the conjugate molecule. Specifically preferred conjugate molecules are conjugate toxins or detectable labels.

TCR fusion and TCR conjugate complexes of the invention comprise a biologically active or effector molecule (terms to be used herein interchangeably) covalently linked to the TCR molecule. As used herein, the term “biologically active molecule” or “effector molecule” is meant an amino acid sequence such as a protein, polypeptide or peptide (also referred to herein as “biologically active polypeptides”]; a sugar or polysaccharide; a lipid or a glycolipid, glycoprotein, lipoprotein or chemical agent that can produce the desired effects as discussed herein. Also contemplated are effector molecule nucleic acids encoding a biologically active or effector protein, polypeptide, or peptide. Thus, suitable molecules include regulatory factors, enzymes, antibodies, or drugs as well as DNA, RNA, and oligonucleotides. The biologically active or effector molecule can be naturally-occurring or it can be synthesized from known components, e.g., by recombinant or chemical synthesis and can include heterologous components. A biologically active or effector molecule is generally between about 0.1 to 100 KD or greater up to about 1000 KD, preferably between about 0.1, 0.2, 0.5, 1, 2, 5, 10, 20, 30 and 50 KD as judged by standard molecule sizing techniques such as centrifugation or SDS-polyacrylamide gel electropheresis. Desired effects of the invention include, for example, either to induce cell proliferation or cell death, initiate an immune response or to act as a detection molecule for diagnostic purposes as determined by the assays disclosed below, including an assay that includes sequential steps of culturing cells to proliferate same, and contacting the cells with a TCR fusion complex of the invention and then evaluating whether the TCR fusion complex inhibits further development of the cells.

Preferred biologically active molecules or effector molecules of the invention may include factors such as cytokines, chemokines, growth factors, protein toxins, immunoglobulin domains or other bioactive proteins such as enzymes. Biologically active molecules that bind to cell surface proteins are preferred proteins of the invention.

Other biologically active molecules or effector molecules of the invention are compounds such as non-protein toxins, cytotoxic agents, chemotherapeutic agents, detectable labels, radioactive materials and such.

Cytokines of the invention are defined by any factor produced by cells that affect other cells and are responsible for any of a number of multiple effects of cellular immunity. Examples of cytokines include but are not limited to the IL-2 family, interferon (IFN), IL-10, IL-1, IL-17, TGF and TNF cytokine families, and to IL-1 through IL-35, IFN-α, IFN-β, IFN-γ, TGF-β, TNF-α and TNFβ.

Chemokines of the invention, similar to cytokines, are defined as any chemical factor or molecule which when exposed to other cells are responsible for any of a number of multiple effects of cellular immunity. Suitable chemokines may include but are not limited to the CXC, CC, C, and CX3C chemokine families and to CCL-1 through CCL-28, CXC-1 through CXC-17, XCL-1, XCL-2, CX3CL1, MIP-1β, IL-8, MCP-1, and Rantes.

Growth factors include any molecules which when exposed to a particular cell induce proliferation and/or differentiation of the affected cell. Growth factors include proteins and chemical molecules, some of which include: GM-CSF, G-CSF, growth factor and stem cell growth factor. Additional growth factors may also be suitable for uses described herein.

Toxins or cytotoxic agents include any substance which has a lethal effect or an inhibitory effect on growth when exposed to cells. More specifically, the effector molecule can be a cell toxin of, e.g., plant or bacterial origin such as, e.g., diphtheria toxin (DT), shiga toxin, abrin, cholera toxin, ricin, saporin, pseudomonas exotoxin (PE), pokeweed antiviral protein, or gelonin. Biologically active molecules of such toxins are well known in the art and include, e.g., DT A chain and ricin A chain. Additionally, the toxin can be an agent active at the cell surface such as, e.g., phospholipase enzymes (e.g., phospholipase C).

Further, the effector molecule can be a chemotherapeutic drug such as, e.g., vindesine, vincristine, vinblastin, methotrexate, adriamycin, bleomycin, or cisplatin.

Additionally, the effector molecule can be a detectably-labelled molecule suitable for diagnostic or imaging studies such as a fluorescent label such as green fluorescent protein, phycoerythrin, cychome, or texas red; or a radionuclide e.g., iodine-131, yttrium-90, rhenium-188 or bismuth-212. See e.g., Moskaug, et al. J. Biol. Chem. 264, 15709 (1989); Pastan, I. et al. Cell 47, 641, 1986; Pastan et al., Recombinant Toxins as Novel Therapeutic Agents, Ann. Rev. Biochem. 61, 331, (1992); “Chimeric Toxins” Olsnes and Phil, Pharmac. Ther., 25, 355 (1982); published PCT application no. WO 94/29350; published PCT application no. WO 94/04689; and U.S. Pat. No. 5,620,939 for disclosure relating to making and using proteins comprising effectors or tags.

A TCR fusion or conjugate complex that includes a covalently linked effector molecule has several important uses. For example, the TCR fusion or conjugate complex can be employed to deliver the effector molecule to certain cells capable of specifically binding the TCR. Accordingly, the TCR fusion or conjugate complex provide means of selectively damaging or killing cells comprising the ligand. Examples of cells or tissue capable of being damaged or killed by the TCR fusion or conjugate complexes include tumors and virally or bacterially infected cells expressing one or more ligands capable of being specifically bound by the TCR. Cells or tissue susceptible to being damaged or killed can be readily assayed by the methods disclosed herein.

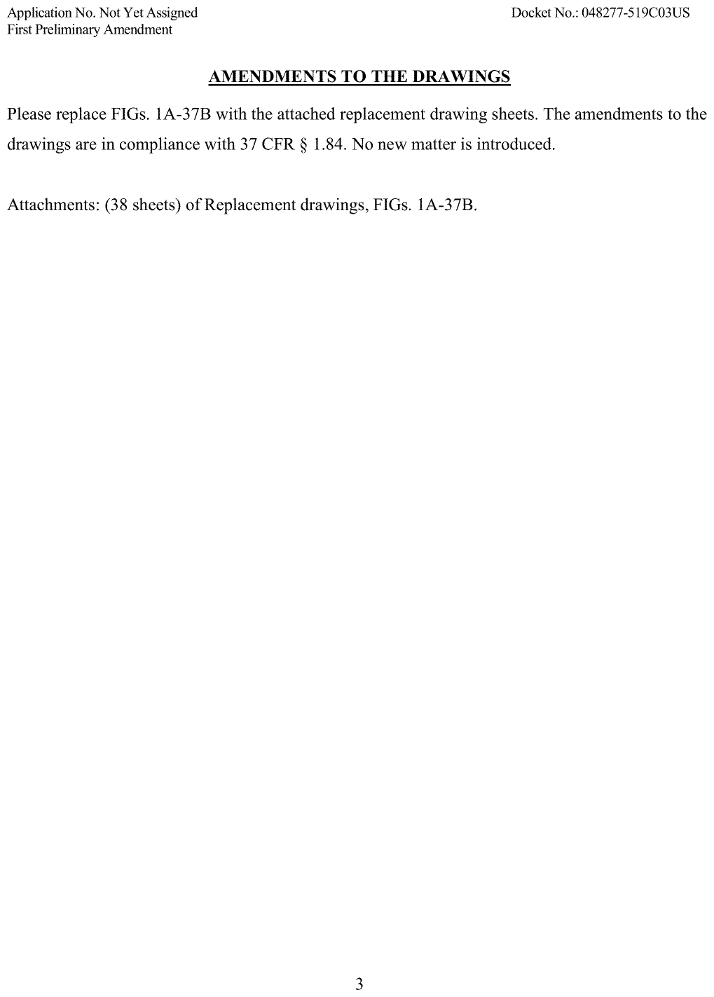

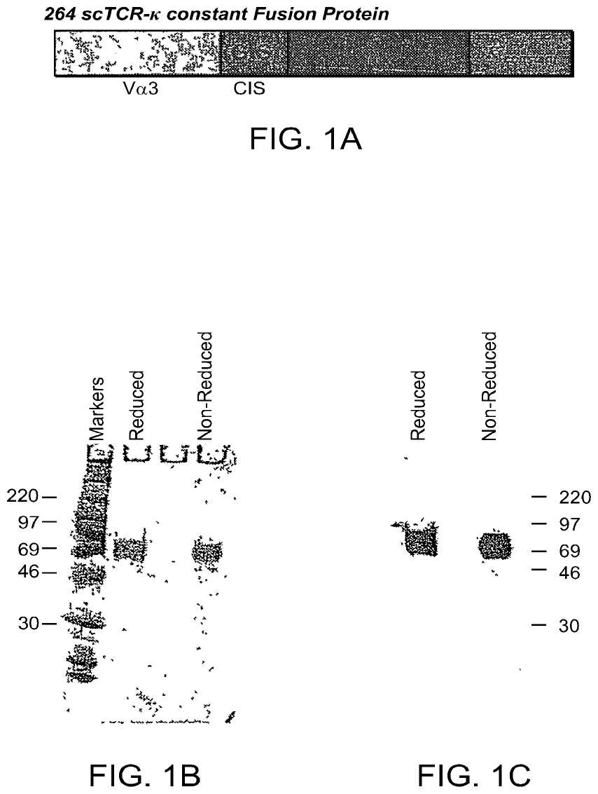

A specific example of a TCR fusion complex fused to an effector molecule is as follows: an sc-TCR such as the 264sc-TCR disclosed below in Examples 5 below can be produced by transfecting mammalian cells with 264scTCR DNA vector illustrated in FIG. 1. The 264scTCR and 149scTCR protein fusion complexes each recognize a processed peptide fragment from human wild-type p53 tumor suppressor protein presented in the context of human HLA antigen; HLA-2.1. The 264scTCR and its peptide ligand have been described in Card et al., Cancer Immunol Immunother (2004) 53: 345. The human p53 (aa264-aa272) peptide sequence (referred to herein as 264 peptide or p264) recognized by 264scTCR is LLGRNSFEV (SEQ ID NO: 1). The human p53 (aa149-aa157) peptide sequence (referred to herein as 149 peptide or p149) recognized by 149scTCR is STPPPGTRV (SEQ ID NO: 2). Expression of tumor suppressor protein p53, is upregulated on malignant cells. It has been shown that 50% of all tumors expressed increased levels of p53 on the surface (Holliston, M. D., et al., Science (1991), 253:49). Therefore, scTCR molecules specific for this epitope could be labeled with a toxin that could than be delivered to the malignant cells expressing the p53 peptide fragment HLA-2.1 ligand. This target specific immunotherapy could be effective at killing only malignant cells. Methods for measuring cytotoxicity in vitro are well-known and include conventional viability assays as described below.

Other fusion molecules of the invention comprises scTCR specific for tumor associated or viral peptide antigens including those derived from MART-1, gp100, MAGE, HIV, Hepatitis A, B or C, CMV, AAV, LCMV, JCV, Influenza, HTLV and other viruses, wherein the scTCR is linked to an effector molecule, either directly or through a linker.

A sc-TCR molecule comprising 2645c-TCR linked to an effector has other important uses. For example, the sc-TCR molecule can be used to selectively kill human breast cancer cells expressing p53 peptide. In vitro studies can be conducted in which the ability of the toxin labeled 264scTCR molecule to kill breast cancer cells is evaluated using a non-radioactive cell cytotoxic assay using a Eu³⁺ release cytotoxicity assay (Bouma, G. J., et al., (1992) Hum. Immunol. 35:85). A sc-TCR molecule comprising a fused effector molecule can be readily tested in vivo. For example, in vitro studies can be carried out by grafting p264/HLA.A21 expressing breast cancer cells into HLA/A2 transgenic mouse. (Theobald, et al., (1995) supra). Toxin labeled 264scTCR molecules can be injected into mice at pre-determined dosages and the effect on tumor size can be measured to indicate efficacy of the sc-TCR molecules. In addition, extension of life can be used as a second criterion to evaluate the efficiency of the novel anti-tumor therapy.

Other suitable effector or tag molecules are known. For example, one tag is a polypeptide bearing a charge at physiological pH, such as, e.g., 6xHIS (SEQ ID NO: 3). In this instance, the TCR fusion or conjugate complex can be purified by a commercially available metallo-sepharose matrix such as Ni-sepharose which is capable of specifically binding the 6xHIS (SEQ ID NO: 3) tag at about pH 6-9. The EE epitope and myc epitope are further examples of suitable protein tags, which epitopes can be specifically bound by one or more commercially available monoclonal antibodies.

In some settings it can be useful to make the TCR fusion or conjugate complexes of the present invention polyvalent, e.g., to increase the valency of the sc-TCR. Briefly stated, the polyvalent TCR protein is made by covalently linking together between one and four proteins (the same or different) by using e.g., standard biotin-streptavidin labeling techniques, or by conjugation to suitable solid supports such as latex beads. Chemically cross-linked proteins (for example cross-linked to dendrimers) are also suitable polyvalent species. For example, the protein can be modified by including sequences encoding amino acid residues with chemically reactive side chains such as Cys or His. Such amino acids with chemically reactive side chains may be positioned in a variety of positions in the fusion protein, preferably distal to the antigen binding region of the TCR. For example, the C-terminus of a C-β chain fragment of a soluble fusion protein can be covalently linked to a protein purification tag or other fused protein which includes such a reactive amino acid(s). Suitable side chains can be included to chemically link two or more fusion proteins to a suitable dendrimer particle to give a multivalent molecule. Dendrimers are synthetic chemical polymers that can have any one of a number of different functional groups of their surface (D. Tomalia, Aldrichimica Acta, 26:91:101 (1993)). Exemplary dendrimers for use in accordance with the present invention include e.g. E9 starburst polyamine dendrimer and E9 combust polyamine dendrimer, which can link cysteine residues.

As used herein, the term “cell” is intended to include any primary cell or immortalized cell line, any group of such cells as in, a tissue or an organ. Preferably the cells are of mammalian and particularly of human origin, and can be infected by one or more pathogens. A “host cell” in accord with the invention can be an infected cell or it can be a cell such as E. coli that can be used to propagate a nucleic acid described herein.

Covalently linking the effector molecule to the TCR peptide in accordance with the invention provides a number of significant advantages. TCR fusion complexes of the invention can be produced that contain a single effector molecule, including such a peptide of known structure. Additionally, a wide variety of effector molecules can be produced in similar DNA vectors. That is, a library of different effector molecules can be linked to the TCR molecule for presentation of infected or diseased cells. Further, for therapeutic applications, rather than administration of an TCR molecule to a subject, a DNA expression vector coding for the TCR molecule linked to the effector peptide can be administered for in vivo expression of the TCR fusion complex. Such an approach avoids costly purification steps typically associated with preparation of recombinant proteins and avoids the complexities of antigen uptake and processing associated with conventional approaches.

As noted, components of the fusion proteins disclosed herein, e.g., biologically active molecules such as cytokines, chemokines, growth factors, protein toxins, immunoglobulin domains or other bioactive molecules and any peptide linkers, can be organized in nearly any fashion provided that the fusion protein has the function for which it was intended. In particular, each component of the fusion protein can be spaced from another component by at least one suitable peptide linker sequence if desired. Additionally, the fusion proteins may include tags, e.g., to facilitate identification and/or purification of the fusion protein. More specific fusion proteins are in the Examples described below. TCR fusion complexes of the invention preferably also include a flexible linker sequence interposed between the TCR protein and the biologically active molecule. The linker sequence should allow effective positioning of the biologically active molecule with respect to the TCR molecule binding groove so that the T cell receptor can recognize presenting MHC-peptide complexes and can deliver the biologically active molecules to a desired site. Successful presentation of the effector molecule can modulate the activity of a cell either to induce or to inhibit T-cell proliferation, or to initiate or inhibit an immune response to a particular site, as determined by the assays disclosed below, including the in vitro assays that includes sequential steps of culturing T cells to proliferate same, and contacting the T cells with a TCR fusion complex of the invention and then evaluating whether the TCR fusion complex inhibits further development of the cells.

The TCR fusion molecules of the invention have the surprising ability to increase either the plasma half life of the molecule (above the plasma half life of the biological effector molecule alone) or the surface residency time for the fusion molecules (above the surface residency time of the biological effector molecule alone) that bind to a cell surface protein, e.g., a cell surface receptor. The TCR fusion molecules of the invention may have the ability to increase the plasma half life of the molecule and increase the surface residency time of the molecule, thereby leading to significant increases in therapeutic potential for the claimed molecules. These molecules also are capable of significantly decreasing the often severe side effects of associated with administration of the component molecules.

In a specific embodiment, the invention provides a soluble humanized scTCR fusion molecules comprising a scTCR specific for amino acid residues 264-272 when presented in the context of HLA-A2.1 and IL-2. The sequence of this fusion molecule is set forth in the figures and the clinical data is set forth in the examples.

In general, preparation of the TCR fusion complexes of the invention can be accomplished by procedures disclosed herein and by recognized recombinant DNA techniques involving, e.g., polymerase chain amplification reactions (PCR), preparation of plasmid DNA, cleavage of DNA with restriction enzymes, preparation of oligonucleotides, ligation of DNA, isolation of mRNA, introduction of the DNA into a suitable cell, transformation or transfection of a host, culturing of the host. Additionally, the fusion molecules can be isolated and purified using chaotropic agents and well known electrophoretic, centrifugation and chromatographic methods. See generally, Sambrook et al., Molecular Cloning: A Laboratory Manual (2nd ed. (1989); and Ausubel et al., Current Protocols in Molecular Biology, John Wiley & Sons, New York (1989) for disclosure relating to these methods.

The invention further provides nucleic acid sequences and particularly DNA sequences that encode the present fusion proteins. Preferably, the DNA sequence is carried by a vector suited for extrachromosomal replication such as a phage, virus, plasmid, phagemid, cosmid, YAC, or episome. In particular, a DNA vector that encodes a desired fusion protein can be used to facilitate preparative methods described herein and to obtain significant quantities of the fusion protein. The DNA sequence can be inserted into an appropriate expression vector, i.e., a vector which contains the necessary elements for the transcription and translation of the inserted protein-coding sequence. A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used. See generally Sambrook et al., supra and Ausubel et al. supra.

In general, a preferred DNA vector according to the invention comprises a nucleotide sequence linked by phosphodiester bonds comprising, in a 5′ to 3′ direction a first cloning site for introduction of a first nucleotide sequence encoding a TCR chain, operatively linked to a sequence encoding an effector molecule.

In most instances, it will be preferred that each of the fusion protein components encoded by the DNA vector be provided in a “cassette” format. By the term “cassette” is meant that each component can be readily substituted for another component by standard recombinant methods. In particular, a DNA vector configured in a cassette format is particularly desirable when the encoded fusion complex is to be used against pathogens that may have or have capacity to develop serotypes.

To make the vector coding for a TCR fusion complex, the sequence coding for the TCR molecule is linked to a sequence coding for the effector peptide by use of suitable ligases. DNA coding for the presenting peptide can be obtained by isolating DNA from natural sources such as from a suitable cell line or by known synthetic methods, e.g. the phosphate triester method. See, e.g., Oligonucleotide Synthesis, IRL Press (M. J. Gait, ed., 1984). Synthetic oligonucleotides also may be prepared using commercially available automated oligonucleotide synthesizers. Once isolated, the gene coding for the TCR molecule can be amplified by the polymerase chain reaction (PCR) or other means known in the art. Suitable PCR primers to amplify the TCR peptide gene may add restriction sites to the PCR product. The PCR product preferably includes splice sites for the effector peptide and leader sequences necessary for proper expression and secretion of the TCR-effector fusion complex. The PCR product also preferably includes a sequence coding for the linker sequence, or a restriction enzyme site for ligation of such a sequence.

The fusion proteins described herein are preferably produced by standard recombinant DNA techniques. For example, once a DNA molecule encoding the TCR protein is isolated, sequence can be ligated to another DNA molecule encoding the effector polypeptide. The nucleotide sequence coding for a TCR molecule may be directly joined to a DNA sequence coding for the effector peptide or, more typically, a DNA sequence coding for the linker sequence as discussed herein may be interposed between the sequence coding for the TCR molecule and the sequence coding for the effector peptide and joined using suitable ligases. The resultant hybrid DNA molecule can be expressed in a suitable host cell to produce the TCR fusion complex. The DNA molecules are ligated to each other in a 5′ to 3′ orientation such that, after ligation, the translational frame of the encoded polypeptides is not altered (i.e., the DNA molecules are ligated to each other in-frame). The resulting DNA molecules encode an in-frame fusion protein.

Other nucleotide sequences also can be included in the gene construct. For example, a promoter sequence, which controls expression of the sequence coding for the TCR peptide fused to the effector peptide, or a leader sequence, which directs the TCR fusion complex to the cell surface or the culture medium, can be included in the construct or present in the expression vector into which the construct is inserted. An immunoglobulin or CMV promoter is particularly preferred.

The components of the fusion protein can be organized in nearly any order provided each is capable of performing its intended function. For example, in one embodiment, the TCR is situated at the C or N terminal end of the effector molecule.

Preferred effector molecules of the invention will have sizes conducive to the function for which those domains are intended. The effector molecules of the invention can be made and fused to the TCR by a variety of methods including well-known chemical cross-linking methods. See e.g., Means, G. E. and Feeney, R. E. (1974) in Chemical Modification of Proteins, Holden-Day. See also, S. S. Wong (1991) in Chemistry of Protein Conjugation and Cross-Linking, CRC Press. However it is generally preferred to use recombinant manipulations to make the in-frame fusion protein.

As noted, a fusion molecule or a conjugate molecule in accord with the invention can be organized in several ways. In an exemplary configuration, the C-terminus of the TCR is operatively linked to the N-terminus of the effector molecule. That linkage can be achieved by recombinant methods if desired. However, in another configuration, the N-terminus of the TCR is linked to the C-terminus of the effector molecule.

Alternatively, or in addition, one or more additional effector molecules can be inserted into the TCR fusion or conjugate complexes as needed.

Preferred fusion and conjugate complexes in accord with the present invention typically include operatively linked in sequence (N to C terminus): 1) a TCR/one or more linker molecules/and a biologically active molecule; 2) TCR/linker molecule/and a biologically active molecule; and 3) TCR/a first linker molecule/a first biologically active molecule subunit/a second linker molecule/and a second biologically active molecule subunit. In addition, one or more protein tags such as EE, HA, Myc, and polyhistidine, particularly 6Xhis (SEQ ID NO: 3), can be fused to the N-terminus of the TCR chains as desired, e.g., to improve solubility or the facilitate isolation and identification of the TCR fusion and conjugate complexes.

The linker sequence is preferably a nucleotide sequence that codes for a peptide that can effectively position the binding groove of the TCR molecule for recognition of a presenting antigen. As indicated above, preferable linker sequence also allow effective positioning of the biologically active molecule with respect to the TCR molecule. As used herein, the phrase “effective positioning of the biologically active molecule”, or other similar phrase, is intended to mean the biologically active molecule linked to a TCR protein is positioned so that the biologically active molecule is capable of interacting with effector cells and modulating the activity of a presenting cell, either to induce cell proliferation, to initiate or inhibit an immune reaction, or to inhibit or inactivate cell development as determined by an assay disclosed below, including the assay that includes sequential steps of culturing cells to proliferate same, and contacting the cells with a TCR fusion complex of the invention and then evaluating whether the TCR fusion complex inhibits further development of the cells.

Preferably the linker sequence comprises from about 1 to 20 amino acids, more preferably from about 1 to 16 amino acids. The linker sequence is preferably flexible so as not hold the biologically active molecule in a single undesired conformation. The linker sequence can be used, e.g., to space the recognition site from the fused molecule. Specifically, the peptide linker sequence can be positioned between the TCR chain and the effector peptide, e.g., to chemically cross-link same and to provide molecular flexibility. The linker is preferably predominantly comprises amino acids with small side chains, such as glycine, alanine and serine, to provide for flexibility. Preferably about 80 or 90 percent or greater of the linker sequence comprises glycine, alanine or serine residues, particularly glycine and serine residues. For a TCR fusion complex that contains a heterodimer TCR, the linker sequence is suitably linked to the β chain of the TCR molecule, although the linker sequence also could be attached to the α chain of the TCR molecule. Alternatively, linker sequence may be linked to both α and β chains of the TCR molecule. For covalently linking an effector molecule peptide to a TCR β chain molecule, the amino sequence of the linker should be capable of spanning suitable distance from the N-terminal residue of the TCR β chain to the C-terminal residue of the effector molecule peptide. When such a β+ peptide chain is expressed along with the α chain, the linked TCR-effector peptide should fold resulting in a functional TCR molecule as generally depicted in FIG. 1. One suitable linker sequence is ASGGGGSGGG (SEQ ID NO: 4) (i.e., Ala Ser Gly Gly Gly Gly Ser Gly Gly Gly (SEQ ID NO: 4)), preferably linked to the first amino acid of the β domain of the TCR. Different linker sequences could be used including any of a number of flexible linker designs that have been used successfully to join antibody variable regions together, see Whitlow, M. et al., (1991) Methods: A Companion to Methods in Enzymology 2:97-105. Suitable linker sequences can be readily identified empirically. Additionally, suitable size and sequences of linker sequences also can be determined by conventional computer modeling techniques based on the predicted size and shape of the TCR molecule.

A number of strategies can be employed to express TCR fusion complexes of the invention. For example, the TCR gene fusion construct described above can be incorporated into a suitable vector by known means such as by use of restriction enzymes to make cuts in the vector for insertion of the construct followed by ligation. The vector containing the gene construct is then introduced into a suitable host for expression of the TCR fusion peptide. See, generally, Sambrook et al., supra. Selection of suitable vectors can be made empirically based on factors relating to the cloning protocol. For example, the vector should be compatible with, and have the proper replicon for the host that is being employed. Further the vector must be able to accommodate the DNA sequence coding for the TCR fusion complex that is to be expressed. Suitable host cells include eukaryotic and prokaryotic cells, preferably those cells that can be easily transformed and exhibit rapid growth in culture medium. Specifically preferred hosts cells include prokaryotes such as E. coli, Bacillus subtillus, etc. and eukaryotes such as animal cells and yeast strains, e.g., S. cerevisiae. Mammalian cells are generally preferred, particularly J558, NSO, SP2-0 or CHO. Other suitable hosts include, e.g., insect cells such as Sf9. Conventional culturing conditions are employed. See Sambrook, supra. Stable transformed or transfected cell lines can then be selected. Cells expressing a TCR fusion complex of the invention can be determined by known procedures. For example, expression of a TCR fusion complex linked to an immunoglobulin can be determined by an ELISA specific for the linked immunoglobulin and/or by immunoblotting.

As mentioned generally above, a host cell can be used for preparative purposes to propagate nucleic acid encoding a desired fusion protein. Thus a host cell can include a prokaryotic or eukaryotic cell in which production of the fusion protein is specifically intended. Thus host cells specifically include yeast, fly, worm, plant, frog, mammalian cells and organs that are capable of propagating nucleic acid encoding the fusion. Non-limiting examples of mammalian cell lines which can be used include CHO dhfr-cells (Urlaub and Chasm, Proc. Natl. Acad. Sci. USA, 77:4216 (1980)), 293 cells (Graham et al., J Gen. Virol., 36:59 (1977)) or myeloma cells like SP2 or NSO (Galfre and Milstein, Meth. Enzymol., 73(B):3 (1981)).

Host cells capable of propagating nucleic acid encoding a desired fusion protein encompass non-mammalian eukaryotic cells as well, including insect (e.g., Sp. frugiperda), yeast (e.g., S. cerevisiae, S. pombe, P. pastoris., K. lactis, H. polymorpha; as generally reviewed by Fleer, R., Current Opinion in Biotechnology, 3(5):486496 (1992)), fungal and plant cells. Also contemplated are certain prokaryotes such as E. coli and Bacillus.

Nucleic acid encoding a desired fusion protein can be introduced into a host cell by standard techniques for transfecting cells. The term “transfecting” or “transfection” is intended to encompass all conventional techniques for introducing nucleic acid into host cells, including calcium phosphate co-precipitation, DEAE-dextran-mediated transfection, lipofection, electroporation, microinjection, viral transduction and/or integration. Suitable methods for transfecting host cells can be found in Sambrook et al. supra, and other laboratory textbooks.

The present invention further provides a production process for isolating a fusion protein of interest. In the process, a host cell (e.g., a yeast, fungus, insect, bacterial or animal cell), into which has been introduced a nucleic acid encoding the protein of the interest operatively linked to a regulatory sequence, is grown at production scale in a culture medium in the presence of the fusion protein to stimulate transcription of the nucleotides sequence encoding the fusion protein of interest. Subsequently, the fusion protein of interest is isolated from harvested host cells or from the culture medium. Standard protein purification techniques can be used to isolate the protein of interest from the medium or from the harvested cells. In particular, the purification techniques can be used to express and purify a desired fusion protein on a large-scale (i.e. in at least milligram quantities) from a variety of implementations including roller bottles, spinner flasks, tissue culture plates, bioreactor, or a fermentor.

An expressed TCR fusion complex can be isolated and purified by known methods. Typically the culture medium is centrifuged and then the supernatant is purified by affinity or immunoaffinity chromatography, e.g. Protein-A or Protein-G affinity chromatography or an immunoaffinity protocol comprising use of monoclonal antibodies that bind the expressed fusion complex such as a linked TCR or immunoglobulin region thereof. The fusion proteins of the present invention can be separated and purified by appropriate combination of known techniques. These methods include, for example, methods utilizing solubility such as salt precipitation and solvent precipitation, methods utilizing the difference in molecular weight such as dialysis, ultra-filtration, gel-filtration, and SDS-polyacrylamide gel electrophoresis, methods utilizing a difference in electrical charge such as ion-exchange column chromatography, methods utilizing specific affinity such as affinity chromatograph, methods utilizing a difference in hydrophobicity such as reverse-phase high performance liquid chromatograph and methods utilizing a difference in isoelectric point, such as isoelectric focusing electrophoresis, metal affinity columns such as Ni-NTA. See generally Sambrook et al. and Ausubel et al. supra for disclosure relating to these methods.

It is preferred that the fusion proteins of the present invention be substantially pure. That is, the fusion proteins have been isolated from cell substituents that naturally accompany it so that the fusion proteins are present preferably in at least 80% or 90% to 95% homogeneity (w/w). Fusion proteins having at least 98 to 99% homogeneity (w/w) are most preferred for many pharmaceutical, clinical and research applications. Once substantially purified the fusion protein should be substantially free of contaminants for therapeutic applications. Once purified partially or to substantial purity, the soluble fusion proteins can be used therapeutically, or in performing in vitro or in vivo assays as disclosed herein. Substantial purity can be determined by a variety of standard techniques such as chromatography and gel electrophoresis.

Truncated TCR fusion complexes of the invention contain a TCR molecule that is sufficiently truncated so the TCR fusion complex can be secreted into culture medium after expression. Thus, a truncated TCR fusion complex will not include regions rich in hydrophobic residues, typically the transmembrane and cytoplasmic domains of the TCR molecule. Thus, for example, for a preferred truncated DR1 TCR molecule of the invention, preferably from about residues 199 to 237 of the β chain and from about residues 193 to 230 of the a chain of the TCR molecule are not included in the truncated TCR fusion complex.

The term “misfolded” as it relates to the fusion proteins is meant a protein that is partially or completely unfolded (i.e. denatured). A fusion protein can be partially or completely misfolded by contact with one or more chaotropic agents as discussed below. More generally, misfolded fusion proteins disclosed herein are representative of a high Gibbs free energy (4G) form of the corresponding native protein. Preferred are native fusion protein which is usually correctly folded, it is fully soluble in aqueous solution, and it has a relatively low AG. Accordingly, that native fusion protein is stable in most instances.

It is possible to detect fusion protein misfolding by one or a combination of conventional strategies. For example, the misfolding can be detected by a variety of conventional biophysical techniques including optical rotation measurements using native (control) and misfolded molecules.

By the term “soluble” or similar term is meant that the fusion molecule and particularly a fusion protein that is not readily sedimented under low G-force centrifugation (e.g. less than about 30,000 revolutions per minute in a standard centrifuge) from an aqueous buffer, e.g., cell media. Further, the fusion molecule is soluble if the it remains in aqueous solution at a temperature greater than about 5-37° C. and at or near neutral pH in the presence of low or no concentration of an anionic or non-ionic detergent. Under these conditions, a soluble protein will often have a low sedimentation value e.g., less than about 10 to 50 svedberg units.

Aqueous solutions referenced herein typically have a buffering compound to establish pH, typically within a pH range of about 5-9, and an ionic strength range between about 2 mM and 500 mM. Sometimes a protease inhibitor or mild non-ionic detergent is added. Additionally, a carrier protein may be added if desired such as bovine serum albumin (BSA) to a few mg/ml. Exemplary aqueous buffers include standard phosphate buffered saline, tris-buffered saline, or other well known buffers and cell media formulations.

The present TCR fusion and conjugate complexes are suitable for in vitro or in vivo use with a variety of cells that are infected or that may become infected by one or more diseases.

As an illustration of the use of the TCR fusion/conjugate therapeutics, a cultured cell can be infected by a pathogen of a single serotype. The infected cell is then contacted by a specified fusion protein in vitro. As discussed previously, the fusion protein is configured so that the toxic domain is presented to the infected cell by the association of the TCR. After providing for introduction of the bioactive molecule to the cell (generally less than about 30 minutes), the cells are allowed to cause a desired effect for a time period of about up to about 2 to 24 hours, typically about 18 hours. After this time, the cells are washed in a suitable buffer or cell medium and then evaluated for viability. The time allotted for cell killing or injury by the fusion protein will vary with the particular effector molecule chosen. However viability can often be assessed after about 2 to 6 hours up to about 24 hours. As will be explained in more detail below, cell viability can be readily measured and quantified by monitoring uptake of certain well-known dyes (e.g., trypan blue) or fluors.

Cells transduced by the fusion molecules of the present invention can be assayed for viability by standard methods. In one approach, cell viability can be readily assayed by measuring DNA replication following or during transduction. For example, a preferred assay involves cell uptake of one or more detectably-labeled nucleosides such as radiolabelled thymidine. The uptake can be conveniently measured by several conventional approaches including trichloroacetic acid (TCA) precipitation followed by scintillation counting. Other cell viability methods include well know trypan blue exclusion techniques.

The TCR molecules of the fusion complexes of the invention suitably correspond in amino acid sequence to naturally occurring TCR molecules, e.g. TCR molecules of a human, mouse or other rodent, or other mammal.

Accordingly, one treatment method of the invention for inhibition of an autoimmune or inflammatory response would include a TCR complex which comprises a T cell receptor effector molecule. Preferably, a “truncated” soluble TCR complex is administered, i.e. the TCR complex does not contain a transmembrane portion. The effector molecule of the administered soluble TCR fusion complex can be selected that are specific for certain cells or specific to generate a desired result. Such effector molecules, including anti-inflammatory molecules or cytokines, can be readily identified and selected by the methods of one of skill in the art. A TCR fusion complex that contains an effector peptide that is a T cell receptor antagonist or partial agonist is particularly useful for treatment of allergies and autoimmune diseases such as multiple sclerosis, insulin-dependent diabetes mellitus and rheumatoid arthritis.

Another treatment method of the invention for induction of an immune response provides for the administration of an effective amount of one or more TCR fusion complexes of the invention. In one embodiment, the effector molecule of the TCR fusion complex is a immunostimulatory molecule such as a cytokine capable of inducing a desired immune response at the location of a diseased cells or tissue that presents the antigen which binds the TCR. The TCR fusion complex may be a truncated form and be administered as a soluble protein as described above. Alternatively, the TCR fusion complex may be full length, i.e. will contain a transmembrane portion. Treatment with these complexes will comprise administration to a mammal an effective amount of a DNA sequence that comprises a DNA vector encoding the full length TCR fusion complex of the invention and a effector molecule.

Different therapies of the invention also may be used in combination as well as with other known therapeutic agents such as anti-inflammatory drugs to provide a more effective treatment of a disorder. For example, immunosuppressive TCR fusion complexes that can be used in combination with anti-inflammatory agents such as corticosteroids and nonsteroidal drugs for the treatment of autoimmune disorders and allergies.

Compounds of the invention will be especially useful to a human patient who has or is suspected of having a malignant disease, disorder or condition, or an infectious disease, disorder or condition. Compounds of the invention will be particularly useful in targeting particular tumor antigens in human patients. Specific examples of diseases which may be treated in accordance with the invention include cancers, e. g. breast, prostate, etc; viral infections, e.g. HCV, HIV, CMV, Hepatitis, JCV, etc. as well as other specific disorders of conditions mentioned herein.

TCR fusion complexes of the invention will be especially useful as therapeutic agents with improved therapeutic activity or safety properties than any component of the fusion complex does alone. Without wishing to be bound by theory, it is believed that increased serum half life and/or cell surface residency time of the TCR fusion complexes will provide improved therapeutic activity or safety properties. Additionally improved therapeutic activity or safety properties could be the result of the activity of the TCR component interact with antigen presented on the diseased cells or tissues, thereby localizing the biological active molecule at those sites. These effects could also include localization or activation of effector cells that interact with the biological active molecule of the TCR fusion complexes. In one embodiment, as described in Examples, TCR fusion complex comprising a tumor antigen-specific TCR and a cytokine molecule exhibited increased serum half life, increased cell surface residency time, increased ability to localize immune cells to the tumor site, less toxicity and/or improved antitumor activity than the cytokine molecule alone or a non-targeted TCR fusion complex.

Additionally, the fusion molecules of the invention significantly decrease the adverse clinical side effects associated with the administration of, for example, cytokine molecules. This allows for the administration of, for example, cytokine-based therapy in an out patient setting, thereby significantly increasing the feasibility of this type to therapy.

Without wishing to be bound by theory, it is believed the multiple and distinct covalently linked compounds of this invention (i.e. at least one identified anti-cancer drug in combination with at least one TCR) can significantly enhance efficacy of the anti-cancer drug, e.g., by increasing targeting of drug to target antigen in subject individuals.

Moreover, by virtue of the covalent linkage, the conjugates of the invention present the anti-cancer drug and the TCR to the subject cell essentially simultaneously, an effect that may not be readily achieved by administering the same compounds in a drug “cocktail” formulation without covalently linking the compounds.

It also has been reported that treatment with treatment with one drug can in turn sensitize a patient to another drug. Accordingly, the essentially simultaneous presentation to the subject cell of an anti-cancer drug and TCR via a conjugate of the invention may enhance drug activity, e.g., by providing synergistic results and/or by enhancing production an immune response.

Administration of compounds of the invention may be made by a variety of suitable routes including oral, topical (including transdermal, buccal or sublingal), nasal and parenteral (including intraperitoneal, subcutaneous, intravenous, intradermal or intramuscular injection) with oral or parenteral being generally preferred. It also will be appreciated that the preferred method of administration and dosage amount may vary with, for example, the condition and age of the recipient.

Components of the invention may be used as part of or in combination with cell-based therapies. TCR fusion complexes of the invention may be used in expansion, activation or targeting of cells prior to or after adoptive cell therapies. In one embodiment, TCR fusion complexes may be used to activate and bind cells following in vitro incubation prior to adoptive transfer into a patient with a disease or disorder. Activated cells bound to the TCR fusion complexes may be effectively localized to the diseased site such that the therapy more effectively treats the disease condition. Cells used in such an approach could include peripheral blood mononuclear cells, T-cells, B-cells, NK cells, LAK cells, stem cells or cells obtained from bone marrow, spleen, liver, lymph nodes, cord blood or other tissues, or derivates of such cells including gene-modified cells or those carrying a transgene. Such cells can be autologous (syngeneic), allogeneic or xenogeneic with respect to the relation between the donor and recipient.

Compounds of the invention may be used in therapy alone or in conjunction with other medicaments such those with recognized pharmacological activity to treat the desired indications. Exemplary medicaments include recognized therapeutics such as surgery, radiation, chemotherapy and other forms of immunotherapy (e.g. vaccines, antibody based therapies). The compounds of this invention can be administered before, during or after such therapies as needed.

While one or more compounds of the invention may be administered alone, they also may be present as part of a pharmaceutical composition in mixture with conventional excipient, i.e., pharmaceutically acceptable organic or inorganic carrier substances suitable for parenteral, oral or other desired administration and which do not deleteriously react with the active compounds and are not deleterious to the recipient thereof. Pharmaceutical compositions of the invention in general comprise one or more TCR fusion complexes of the invention or DNA constructs coding for such fusion complexes together with one or more acceptable carriers. The carriers must be “acceptable” in the sense of being compatible with other ingredients of the formulation and not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

For parenteral application, particularly suitable are solutions, preferably oily or aqueous solutions as well as suspensions, emulsions, or implants, including suppositories. Ampules are convenient unit dosages.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

Therapeutic compounds of the invention also may be incorporated into liposomes. The incorporation can be carried out according to known liposome preparation procedures, e.g. sonication and extrusion. Suitable conventional methods of liposome preparation are also disclosed in e.g. A. D. Bangham et al., J. Mol. Biol., 23:238-252 (1965); F. Olson et al., Biochim. Biophys. Acta, 557:9-23 (1979); F. Szoka et al., Proc. Nat. Acad. Sci., 75:4194-4198 (1978); S. Kim et al., Biochim. Biophys. Acta, 728:339-348 (1983); and Mayer et al., Biochim. Biophys. Acta, 858:161-168 (1986).

The invention also provides methods for invoking an immune response in a mammal such as a human, including vaccinating a mammal such as a human against an infectious agent or a targeted disorder such as cancer.

These methods comprise administering to a mammal an effective amount of a DNA sequence that comprises a DNA vector that codes for a TCR fusion complex of the invention. Preparation of expression vectors of TCR fusion complexes is described above and in the Examples which follow. Methods for administration of plasmid DNA, uptake of that DNA by cells of the administered subject and expression of protein has been reported. See Ulmer, J. B., et al., Science (1993) 259: 1745-1749.

DNA vectors that encode TCR fusion complexes of the invention are suitably administered to a mammal including a human preferably by intramuscle injection. Administration of cDNA to skeletal muscle of a mammal with subsequent uptake of administered expression vector by the muscle cells and expression of protein encoded by the DNA has been described by Ulmer et al. and represents an exemplary protocol [Ulmer, J. B., et al., Science 259: 1745-1749]. The optimal dose for a given therapeutic application can be determined by conventional means.

In addition to treatment of human disorders, TCR fusion and conjugate complexes of the invention and DNA constructs of the invention that encode such fusion complexes will have significant use for veterinary applications, e.g., treatment of disorders of livestock such as cattle, sheep, etc. and pets such as dog and cats.

In an exemplary embodiment, the fusion molecules of the inventions can be administered by intravenous bolus for the first 4 days (cycle 1), followed by a 10-day rest period of no treatment (rest period) and then once daily as an intravenous bolus for an additional 4 days (cycle 2). The dosing regime has a starting dose of 0.015 mg/kg (equivalent to approximately 50,000 IL-2 IU/kg), 0.04 mg/kg or 0.08 mg/kg Further administration can be as necessary and as determined by a physician.

In specific embodiments, the fusion molecules of the invention halt tumor growth in a subject. In other embodiments, the fusion molecules decrease tumor volume by at least 10, 15, 20, 25, 30, 35, 40, 45, 50, 55, 60, 65, 70, 75, 80, 85, 90 or more percent.

The administration of the fusion molecules of the invention have also shown no or very small amounts of serum TNF-α in the treated subjects' blood samples. TNFα is known to be induced in patients' sera following rhIL-2 treatment. and is suspected of being the causative agent of many of the adverse side effects associated with cytokine therapy. Accordingly, in one aspect of the invention, the fusion molecules do not increase the amount of TNFα in a subjects blood.

It will be appreciated that actual preferred amounts of a given TCR fusion complex of the invention or DNA construct coding for same used in a given therapy will vary according to the particular active compound or compounds being utilized, the particular compositions formulated, the mode of application, the particular site of administration, the patient's weight, general health, sex, etc., the particular indication being treated, etc. and other such factors that are recognized by those skilled in the art including the attendant physician or veterinarian. Optimal administration rates for a given protocol of administration can be readily determined by those skilled in the art using conventional dosage determination tests conducted e.g. with regard to the foregoing guidelines and the assays disclosed herein.

All documents mentioned herein are fully incorporated herein by reference in their entirety. The following non-limiting examples are illustrative of the invention.

### Example 1

### Construction of 264 Single-Chain (Sc) TCR

The T cell clone, 264, recognizes a peptide fragment (aa 264-272; LLGRNSFEV (SEQ ID NO: 1)) of the human wild-type tumor suppresser protein p53 restricted by HLA-A2.1. The T cell receptor gene was cloned into a three domain single-chain format previously shown to produce soluble TCR and functional receptor molecules.

In brief, mRNA was isolated from the T cell clone and cDNA was made using the Marathon cDNA Amplification Kit (Clontech). Sequencing of cDNA clones identified two distinct V alpha chains (Valpha 3 and V alpha 13) and a single V beta chain (V beta 3). The cDNA was used as a template in polymerase chain reaction (PCR) with primers KC228 and KC229 or KC226 and KC227 to produce 5′SfiI-3′SpeI V alpha 3 or V alpha 13 fragments respectively. The same DNA was then used as a PCR template with primers PRIB4 and KC176 to generate a 5′XhoI-3′XmaI V beta C beta chain fragment. The C beta chain was truncated just before the cysteine residue at amino acid 127 of the full length C beta chain.

The alpha and beta chain fragments were cloned into the pGEM-T Easy Vector System (Promega) for DNA sequence determination. Correct fragments were restriction digested and cloned into expression vector pKC60 (described previously in pending U.S. patent application Ser. No. 08/813,731) to create two V alpha-(G₄S)₄ ((G₄S)₄ disclosed as SEQ ID NO: 5)V beta C beta scTCR molecules, 264-A (with V alpha 3) and 264-B (with V alpha 13).

The DNA constructs described above (264-A and 264-B) were reamplified by PCR with primers ET-TCRF1 and KC170 or ET-TCRF2 and KC170, respectively, to generate 5′AgeI-3′ClaI DNA fragments. The fragments were cloned into the pGEM-T Easy Vector System for DNA sequence determination.

The 5′AgeI-3′ClaI fragments were then used as the template DNA in PCR with primers KC232 and KC208 or KC231 and KC208, respectively, to produce 5′AgeI-3′HpaI DNA fragments for cloning into the CD3 zeta fusion molecule (described below) and eventually the 264scTCR/IL-2 fusion molecule (described below).

### Example 2

### Construction of the CD3 Zeta Fusion Vector

To determine which of the two V alpha chains was functional, both the 264-A and 264-B scTCR were expressed as CD3 zeta fusion molecules.

Construction of a shuttle vector has been previously described in pending U.S. application Ser. No. 09/422,375.

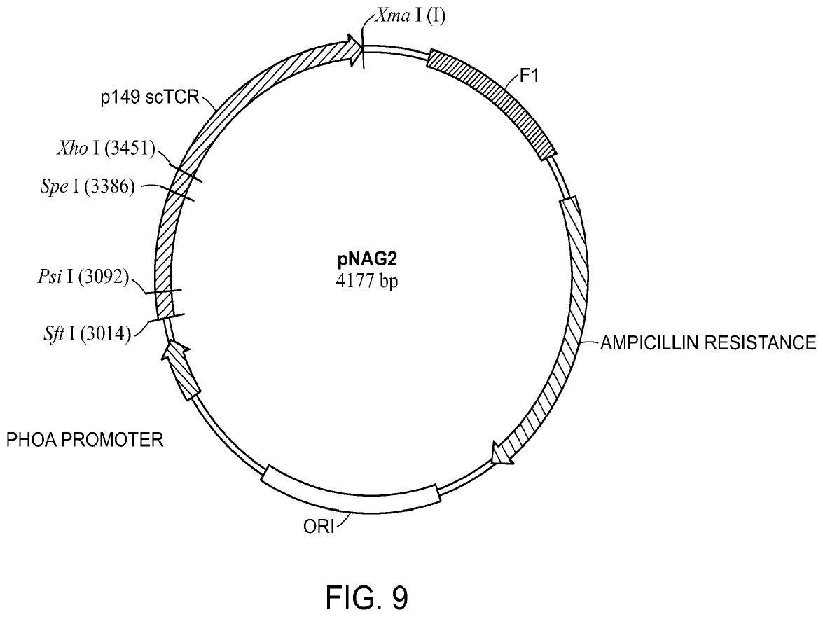

Briefly, alpha and beta chain TCR fragments were cloned into the into the expression vector pKC60 to create a V alpha-(G₄ S)₄ V beta C beta scTCR molecule. The new vector was named pNAG2 (FIG. 9). pNAG2 was then reamplified by PCR with primers KC203 and KC208 to generate a 5′AgeI-3′HpaI/BspEI/NruI/ClaI DNA fragment. The scTCR fragment was cloned into the pGEM-T Easy Vector System and this new pGEM-based vector was then used as a “shuttle vector” for introduction of other DNA fragments to create a bispecific sc molecule.

Sc-Fv DNA was then restriction digested and cloned into the “shuttle vector” downstream of the scTCR. To connect the scTCR and scSc-Fv together as a single-chain fusion protein, the “shuttle vector” was digested with the appropriate restriction enzymes to drop out the previous linker DNA fragment and allow for ligation of linker sequences between the scTCR and the Sc-Fv.

In the “shuttle vector” design outlined above, a stop codon and splice site were introduced between the NruI and ClaI restriction sites as part of the PCR amplification of the scTCR with “back” primer KC208. To aid in downstream purification of the bispecific sc protein, a set of annealed oligos (KC237 and KC238) was designed to introduce a 3′ EE tag (EEEEYMPME (SEQ ID NO: 6)) with stop codon and splice site. The annealed oligo pair was cloned 5′NruI-3′ClaI into the “shuttle vector” already encoding for the complete bispecific sc molecule.

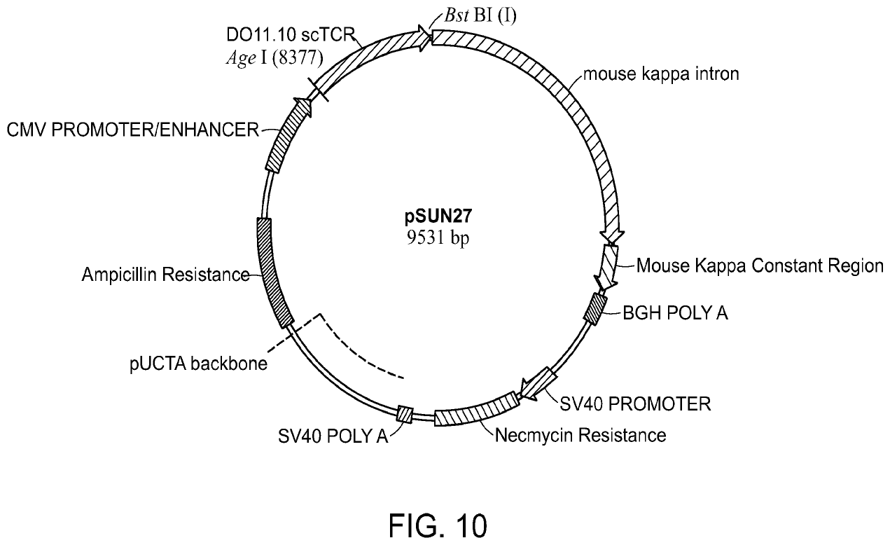

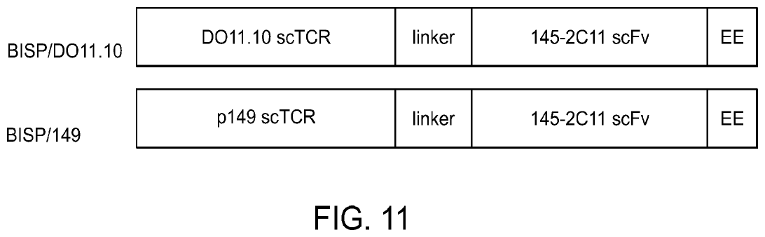

After cloning the scTCR, Sc-Fv, linker, and tag DNA fragments into the “shuttle vector” to complete the bispecific sc molecule design, the DNA was restriction digested (AgeI-ClaI) and cloned into the mammalian cell expression vector pSUN27 (FIG. 10) (previously described in the pending U.S. application Ser. No. 08/943,086 to create pBISP/149 (FIG. 11).

### Construction of the CD3 Zeta Fusion Vector

In brief, murine cDNA was used as the template in polymerase chain reaction (PCR) with primers KC312 and KC304 to produce a 5′HpaI-3′ClaI murine CD3 zeta fragment.

The murine CD3 zeta fragment was cloned into the pGEM-T Easy Vector System for DNA sequence determination. The correct fragment was restriction digested and cloned into the “shuttle vector”, effectively removing the existing linker, scFV, and EE tag.

After cloning the CD3 zeta gene into the “shuttle vector”, the DNA was digested AgeI-HpaI to allow for ligation with the 264-A and 264-B scTCR fragments (described above), creating two new scTCR/CD3 zeta fusions. Lastly, the new DNA preparations were restriction digested (AgeI-ClaI) and cloned into the mammalian cell expression vector pSUN28 (pBISP/DO II. 10 vector), FIG. 11 previously described in pending patent U.S. application Ser. No. 09/422,375.

### Example 3

Expression of 264 scTCR/CD3 Zeta Fusion Molecules

Jurkat cells were prepared for transfection by washing with cold DPBS. The cells were resuspended in DPBS and mixed with 20 ug of PvuI linearized 264-A/CD3 zeta or 264-B/CD3 zeta DNA. After five minutes on ice, the cells were electroporated using a Gene Pulser (BioRad) set to deliver one pulse of 250 volts, 960 u Fd or 0.25 u Fd. The pulsed cells were placed on ice for five minutes. The cells were diluted into 10 ml of 10% IMDM medium (IMDM, 10% FBS, 2 mM glutamine) and grown in a T-25 cm²TC flask overnight at 37 C with 5% CO₂. The next day, the cells were plated in 96 well plates with selective medium (10% IMDM plus 1.0 mg/ml G418). After 1 week, the concentration of G418 was increased to 2 mg/ml. The growing colonies were refed approximately two weeks after transfection and screened about one week later.

The transfected Jurkat cells were screened for surface expression of scTCR using flow cytometry analysis. Positive transfectants were identified by staining with a fluorescent-tagged mAb (H57-597) which detects a portion of the C beta domain of murine TCR.

### Example 4

Identification of the Correct 264 scTCR V Alpha Domain

Transfected Jurkat cells which expressed either the 264-A or 264-B version of the CD3 zeta fusion molecule were used in a cell activation assay. In the assay, the HLA-A2 presenting cell line T2 was used as the APC. The T2 cells were loaded with 264 peptide (or irrelevant peptide) overnight at 37 C with 5% CO2. The following day, the transfected Jurkat lines were added and allowed to interact with the peptide-pulsed APCs overnight.

Specific stimulation of the transfectants by p264-loaded APCs was assessed using an IL-2 ELISA. An anti-human IL-2 mAb was coated passively overnight on a 96 well plate. The plate was washed and blocked with 10% FBS/DPBS for 1 hour. The blocking reagent was flicked out and supernatants from the assay were added to the plate for 1 hour at 37 C. After washing, the bound IL-2 was detected using another anti-IL-2 mAb conjugated to biotin. Following 45 minutes at 37 C, the plate was washed and strepavidin-HRP was added for 15 minutes. Finally, the plate was washed and developed using ABTS substrate. Absorbance was read at 405 nm.

Based on the cell activation assay, the V alpha 3 domain is functional. Only the 264-A molecule was stimulated to produce IL-2 in the presence of p264 peptide-loaded APCs.

### Example 5

Construction of the 264 scTCR/IL-2 Fusion Molecule

To generate the scTCR/IL-2 fusion molecule, the human IL-2 gene needed to be cloned into a DNA expression vector.

In brief, total RNA was isolated from human Jurkat cells using the Mini Total RNA Kit (Qiagen) and Qiashredder (Qiagen). The RNA was concentrated and used in a reaction with reverse transcriptase and a specific back primer, KC328B, to generate cDNA. The cDNA was used as the template in PCR with primers KC327B and KC328B to produce a 5′BspI-3′NruI human IL-2 gene fragment.

The human IL-2 fragment was cloned into the pGEM-T Easy Vector System for sequence determination. The correct fragment was restriction digested and cloned into the “shuttle vector”, effectively removing the existing scFv gene.

The “IL-2 modified shuttle vector” was then restriction digested (BspI-NruI) and the scTCR (described above) was ligated in to complete the scTCR/IL-2 design. Finally, the DNA was cut AgeI-ClaI and cloned into the mammalian cell expression vector pSUN28.

### Example 6

Construction of the 149 scTCR/IL-2 Fusion Molecule

To create the 149 scTCR (described in detail in patent application Ser. No. 09/422,375) version of the IL-2 fusion, the 149 scTCR was cut out of the “shuttle vector” (see example 7 of patent application Ser. No. 09/422,375) as an 5′AgeI-3′HpaI fragment and then ligated into the “IL-2 modified shuttle vector” (described above). The 149 scTCR/IL-2 fragment was then restriction digested (AgeI-ClaI) and cloned into the mammalian cell expression vector pSUN28.

### Example 7

Expression of the scTCR/IL-2 Fusion Molecules

CHO cells were prepared for transfection by washing with cold DPBS. The cells were resuspended in DPBS and mixed with 20 ug of PvuI linearized 264 scTCR/IL-2 or 149 scTCR/IL-2. After five minutes on ice, the cells were electroporated using a Gene Pulser set to deliver one pulse of 250 volts, 960 u Fd or 0.25 u Fd. The pulsed cells were placed on ice for five minutes. The cells were diluted into 10 ml of 10% IMDM medium (IMDM, 10% FBS, 2 mM glutamine) and grown in a T-25 cm² TC flask overnight at 37 C with 5% CO₂. The next day, the cells were plated in 96 well plates with selective medium (10% IMDM plus 1 mg/ml G418) and refed after approximately 7 days.

Transfectants were screened for expression of soluble fusion molecules in an ELISA assay format. An anti-human IL-2 antibody was passively coated overnight onto a 96 well plate. On assay day, the plates were blocked with 10% FBS/PBS for one hour. The wells were washed and supernatant from the transfectants was added to the plate. After incubating and washing, biotinylated anti-C beta mAb H57-597 (cell line was purchased from ATCC) was added to the plate, followed by washing and incubation with streptavidin-HRP. Positive wells were identified by the addition of TMB substrate, quenched with 1N sulfuric acid, and read at an absorbance of 450 nM. A small number of positive clones were selected for expansion and limiting dilution cloning was carried out to establish stable transfected cell lines.

Transfectants could also be screened for the expression of fusion molecules in an ELISA assay format using mAbs which specifically recognize each of the scTCRs followed by detection with biotinylated anti-C beta mAb and streptavidin-HRP. For the 149scTCR fusion molecule, a conformational mAb to the V alpha domain (B20.1, Pharmagen) was used as the coating antibody. The 264scTCR fusion molecule could be detected using the a conformational mAb to its V beta domain (KJ25, Pharmingen).

### Example 8

Purification of scTCR/IL-2 Fusion Protein.

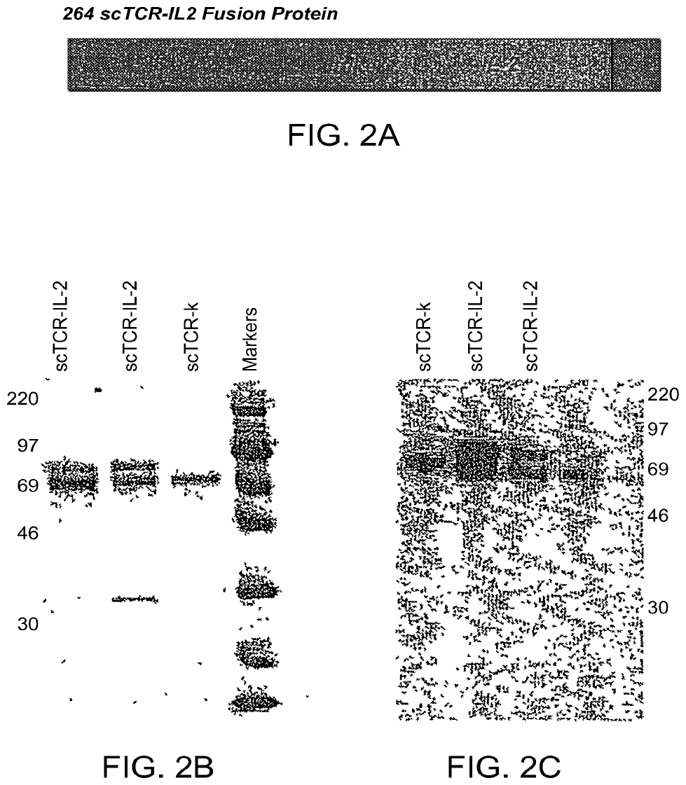

TCR/IL-2 fusion proteins were purified from transfectant supernatant using standard affinity chromatography methods. The fusion proteins were applied to an anti-TCR Cβ mAb-specific CNBr-coupled agarose column for enrichment. In brief, supernatant was passed over the column bed one time. After washing with PBS, the bound protein was eluted off the column by the addition of low pH glycine buffer (pH3.0) and immediately neutralized by the addition of a 1 to 10 dilution of 2M Tris, pH 8.0. The purified protein was then buffered exchanged into PBS using a 30 kD MW cut-off concentration unit. The final protein concentration was determined by an OD280 reading. Coomassie blue staining of the purified protein (FIG. 2) and immunoblot analysis of the purified protein (FIG. 2) shows enrichment for the 264 scTCR-IL-2 fusion protein.

### Example 9

### CTLL-2 Proliferation Assay.

The IL-2 dependent murine T cell line, CTLL-2, was used to evaluate the IL-2 activity of the 264 scTCR-IL-2 fusion protein using a non-radioactive cell proliferation assay. The 264 scTCR-C-kappa fusion protein was used in the assay as a negative control. Briefly, CTLL-2 cells used for the assay were seeded at 10⁴ cells/ml and allowed to grow for 48 hrs in order to deplete residual IL-2. Over the 48 hr period the cells grew to a density of 1.15×10⁵ cells/ml. Cells were then harvested and washed several times using 10% IMDM (w/o IL-2) to remove any remaining IL-2. A 96 well flat bottom plate was used for the assay. First, 50 ul of media (10% IMDM), media w/IL-2 or purified 264scTCR-IL-2 or 264scTCR-C-kappa fusion protein was added to each well. The CTLL-2 cells were added to wells at 10⁵ cells/50 ul. An IL-2 standard was run on the same plate. The plate was incubated overnight at 37° C. with 5% CO2. The following day, cell death was clearly evident using a microscope. Cell proliferation/viability was assessed using the Celltiter Assay. The CellTiter96 assay is an aqueous non-radioactive cell proliferation assay marketed by Promega Corp. The assay is composed of solutions of a novel tetrazolium compound, MTS, and an electron coupling reagent; PMS. MTS is bioreduced by cells into a formazan that is soluble in tissue culture medium. The absorbance of the formazan at 490 nm can be measured directly from the 96 well assay plates without additional processing. The conversion of MTS into the aqueous soluble formazan is accomplished by dehydrogenase enzymes found in metabolically active cells. The quantity of formazan product measured by the amount of 490 nm absorbance is directly proportional to the number of living cells in culture. The 264scTCR/IL-2 and 264scTCR-C-kappa fusion proteins were tested for activity by diluting from 1.25 μg/well to 0.0098 μg/well.

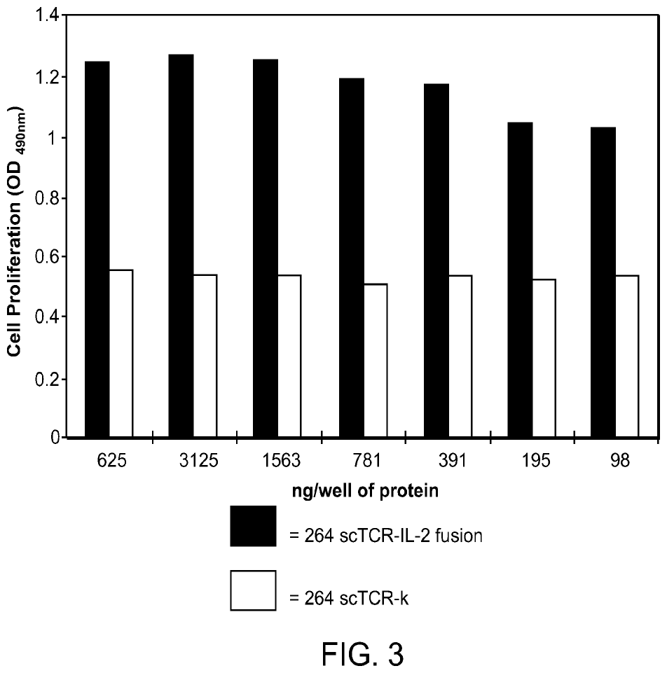

The results from one experiment are shown in FIG. 3. The IL-2 dependent murine T cell line, CTLL-2, was used to evaluate the IL-2 activity of the 264 scTCR-IL-2 fusion protein using a non-radioactive cell proliferation assay. The 264 scTCR-C-kappa fusion protein was used in the assay as a negative control.

### Example 10

Staining of Peptide-Pulsed Cells with the 264 scTCR-IL-2 Fusion Protein Demonstrates a Functional scTCR.

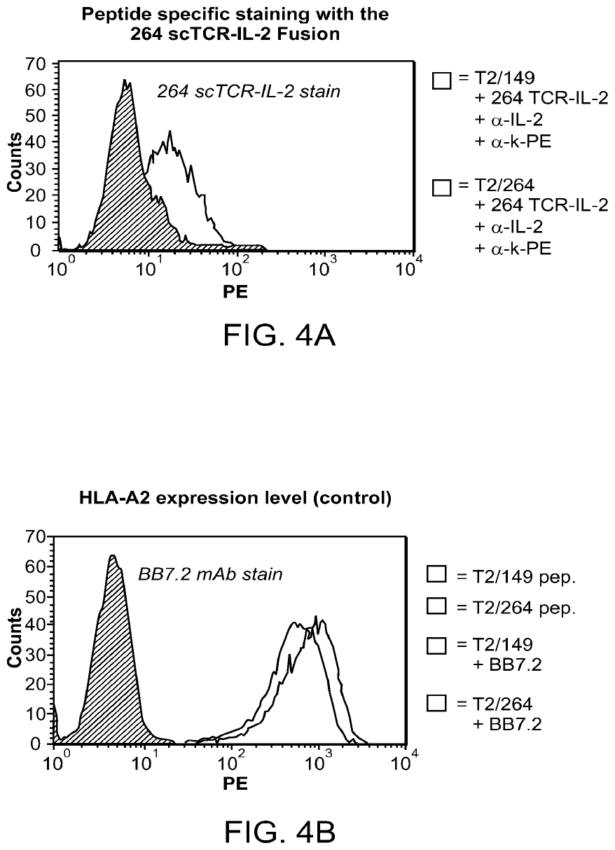

Flow cytometry and immunofluoresence staining were used to show direct binding of the fusion protein via its TCR to peptide/HLA-A2 complexes on the surface of the human B lymphoid cell line T2 (FIG. 4)
A) Staining of 149 or 264 peptide pulsed T2 cells with 0.5 μg (10 μg/ml) of 264 scTCR-IL-2 fusion protein. The 264 scTCR-IL-2 fusion protein binds specifically to T2 cells displaying the 264 peptide but not the 149 peptide.
B) T2 cells were pulsed with 50 μg of either 149 or 264 peptide. To evaluate A2 loading of each peptide, the cells were stained with the HLA-A2 specific mAb BB7.2 (0.05 μg) after overnight incubation of the cells with peptide. The results from these experiments show an equivalent level of HLA-A2 surface expression on T2 cells pulsed with either peptide indicating efficient HLA-A2 binding of both peptides.

### Example 11

Cell-Cell Conjugation Mediated Specifically by the 264 scTCR-IL-2 Fusion Protein.

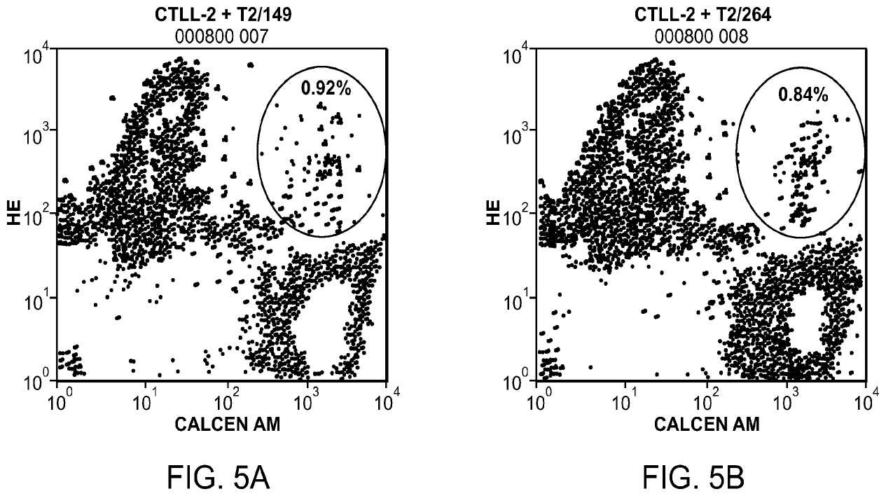

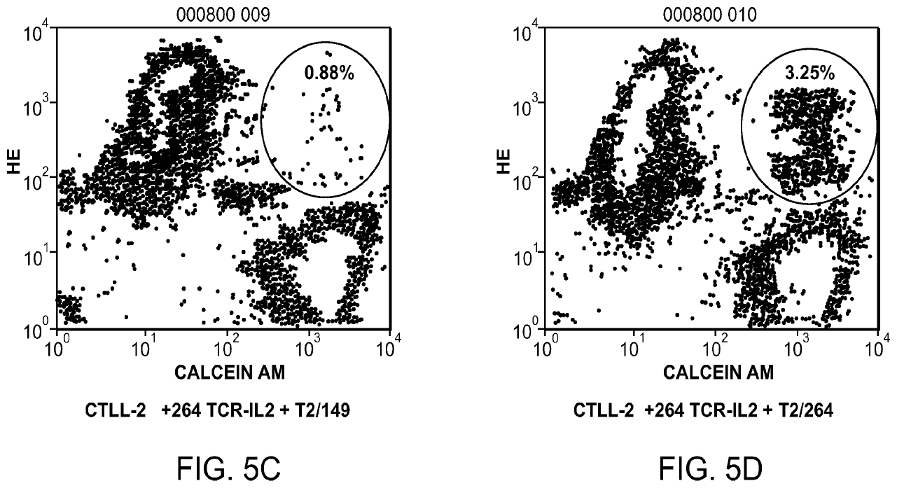

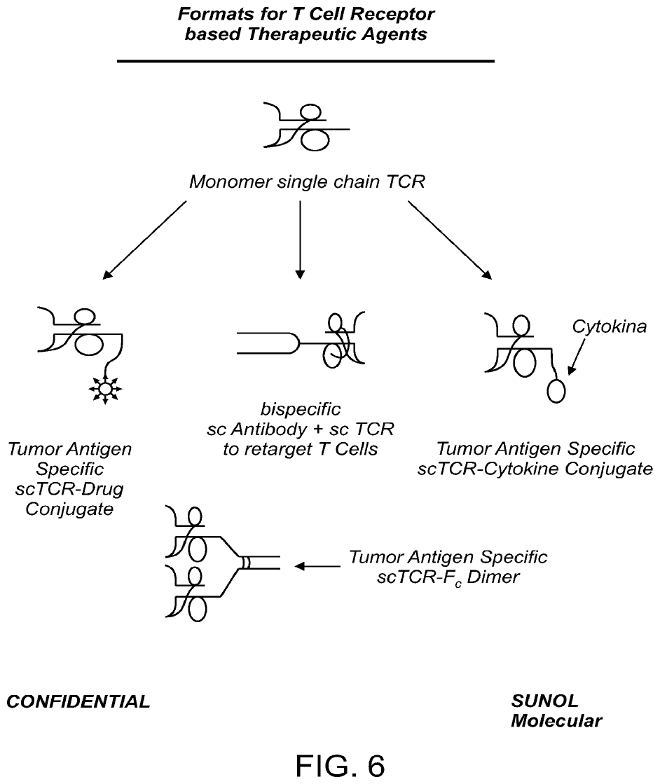

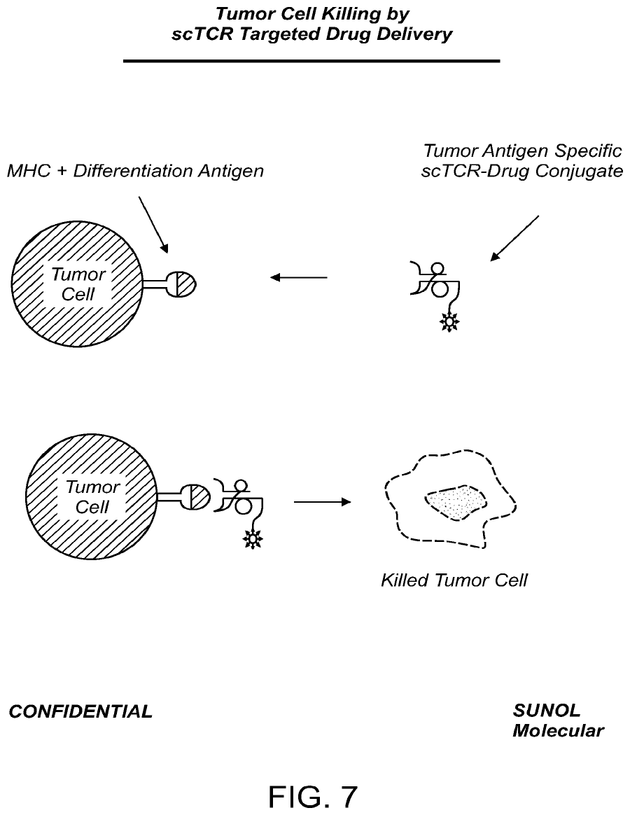

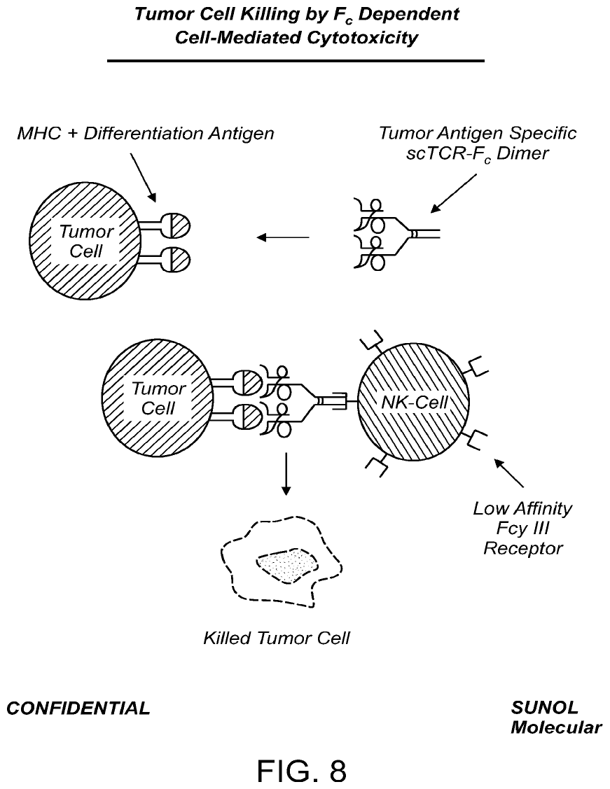

In this experiment, T2 cells pulsed with either the 149 or 264 peptide. CTLL-2 cells were hydrodiethidium (HE) labeled and incubated for 20 minutes at RT with an equal number of calcein-AM labeled T2 cells pulsed with either 50 μg of 149 or 264 peptide. FIG. 5 shows conjugation between cells when 1 μg of fusion protein was added to the incubation mixture containing CTLL-2 cells and 264 peptide-loaded T2 cells (A; 3.25%). In contrast, conjugate formation was not observed with the mixture that included the 149 peptide pulsed T2 cells (B; 0.88%). Cell samples were washed one time before analysis on the flow cytometer.

### Example 12

Construction of scTCR/IgG (murine) Fusion Molecules, have been previously described in pending U.S. application Ser. No. 09/422,375, incorporated herein by reference.

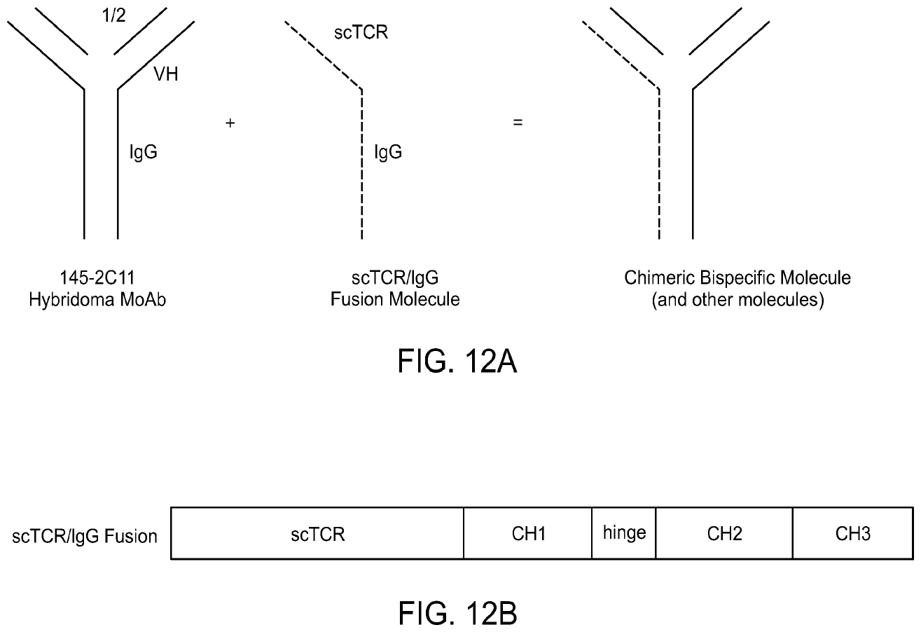

There has been recognition that the expression of the 145-2CII scSc-Fv alone, i.e. not as part of a bispecific sc molecule, is very low. Without wishing to be bound to theory, the low level of sc-Fv expression may be a limiting factor in the expression of bispecific molecules. Native 145-2C 11 hybridoma cell line was used as antibody source and cells were transfected with scTCR fused with murine IgG2b heavy chain (FIG. 12). The transfected hybridoma cell line should secrete some 145-2C11/scTCR chimeric molecules if the host's hamster IgG can pair efficiently with murine IgG2b heavy chain.

To clone the 149scTCR as an IgG fusion, an internal EcoRI restriction site was first mutated using site-directed mutagenesis. Briefly, a pair of complimentary oligonucleotides, KC293 and KC294, were designed containing the desired mutation. The pNAG2 DNA construct was amplified by PCR with the primers using Pfu DNA polymerase. The resulting PCR product was digested with DpnI which digests the parental DNA template, leaving the mutated DNA intact. The mutated scTCR DNA was sequenced and then reamplified by PCR with primers KC276 and KC268 to generate a 5′NruI-3′EcoRI DNA fragment. The mutated scTCR DNA was cloned into the pGEM-T Easy Vector System for DNA sequence determination. The correct scTCR DNA was restriction digested and cloned into the mammalian cell expression vector pSUN7 to create the p149 scTCR/IgG fusion molecule.

Construction of DO 11.10 scTCR/IgG Fusion Molecule

The pKC60 DNA construct was reamplified by PCR with primers KC275 and KC268 to generate a 5′NruI-3′EcoRI DNA fragment. The scTCR fragment was cloned into the pGEM-T Easy Vector System for DNA sequence determination. The correct scTCR DNA was restriction digested and cloned into the mammalian cell expression vector pSUN7 to create the DO 11.10 scTCR/IgG fusion molecule (See FIGS. 12A/12B).

### Construction of the Murine IgG2b Expression Vector

The construction of the murine IgG2b (heavy chain) expression vector was as follows. The backbone of the vector was the plasmid pCDNA3 (Invitrogen). The plasmid was cut with HindIII and XhoI and a “light chain polylinker” DNA fragment was inserted to create the starting “light chain vector” pCDNA3.LCPL. This linker contained the restriction sites HindIII, KpnI, ClaI, PmII, EcoRV, XmaI, BarnHI, and XhoI to facilitate subsequent cloning steps. A SmaI-BcII DNA fragment containing a light chain leader, mouse anti-CKMB kappa light chain genomic fragment, and 3′ UTR was cloned into the EcoRV-BarnHI sites of pCDNA3.LCPL. Mutagenesis was then performed to eliminate an NruI MluI, and BstBI site and to introduce an NheI and BarnHI site to create the plasmid pCDNA3mut.LCPL.LCVK.

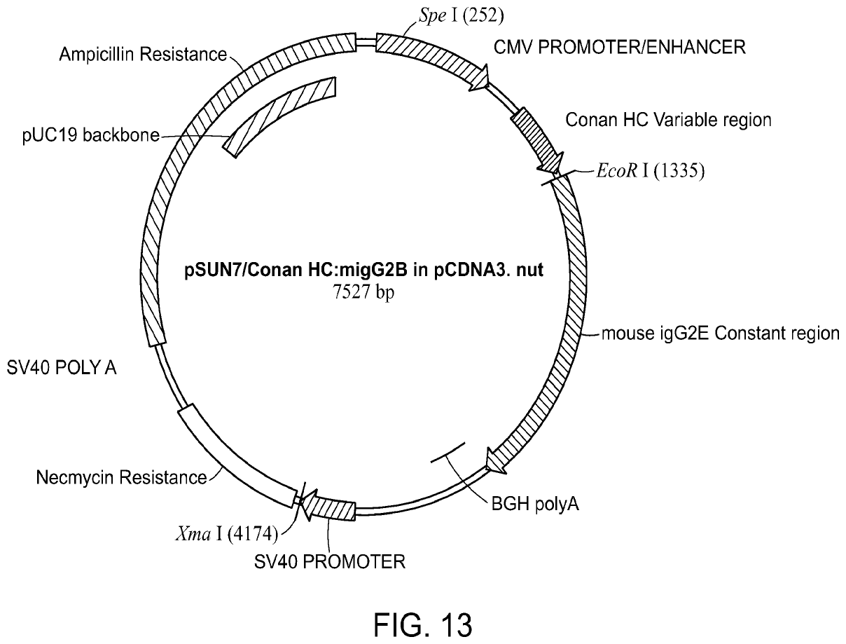

The “heavy chain vector” pCDNA3mut.HCPL was constructed from the pCDNA3mut.LCPL.LCVK plasmid by replacing the light chain expression region (HindIII-XhoI) with a “heavy chain polylinker” consisting of restriction sites HpaI, BspEI, EcoRV, KpnI, and XhoI. This plasmid was digested with EcoRv and KpnI. A SmaIKpnI digested DNA fragment containing a heavy chain leader and an anti-CKMB IgG2b mouse heavy chain genornic fragment (see Near et al., Molecular Immun., 1990) was then ligated into the EcoRV-KpnI digested plasmid. A KpnI-SalI oligonucleotide fragment containing a 3′UTR and a NotI site upstream of the SalI site was subsequently cloned into the KpnI-XhoI digested plasmid (knocking out the XhoI site) to create the plasmid pCDNA3mut.HCPL.HCV2b, also known as the murine IgG2b expression vector pSUN7 (FIG. 13).

### Example 13—Construction of scTCR/IgG (Human) Fusion Molecules. (Cloning of scTCR 264 into pJRS355 and Expression as an IgG1 Fusion)

A DNA preparation of the 264 scTCR provided by Kim Card was used as a template for the PCR amplification of this scTCR construct. Reamplification of the scTCR was carried out using the primer set of 264 TCR1s and KC268. The newly designed 264 TCR1s sequence reads as follows, 5′-TTTCgTACgTCTTgTCCCAgTCAgTgACgCAgC-3′ (SEQ ID NO: 7). This oligonucleotide has been designed with a Bsi WI restriction endonuclease site and a B6.2 leader. Takara ExTag polymerase was used in the amplification reaction following standard PCR protocol. The amplification profile was as follows, 96° C./2 min for 1 cycle; 96° C./30 sec, 62 C/15 sec, 72 C/30 sec for 5 cycles; and 96 C/30 sec, 68 C/1 min for 30 cycles. The proper MW (˜1.3 Kb) DNA band was gel purified following the Clonetech protocol and cloned into Promega's pGem-T easy vector. After ligation and transformation into XL1-Blue cells, six clones were picked and screened by diagnostic PCR using two primers, KC 285 and KC 288, provided by Kim Card. Five clones out of six, produced a DNA band of the proper MW. DNA sequence analysis was carried out on two clones, scTCR264/pGem A and B, with each clone found to be correct. Double digest (Bsi WI and Eco RI) reactions were set up for clones A and B. The proper DNA fragments were gel purified and pooled together. The purified 264 scTCR was cloned into a previously prepared pJRS355 vector DNA. After ligation and transformation into XL1-Blue cells, two colonies were picked (A2 and B1). An Alw NI digest of their DNA showed the proper restriction pattern. Transient transfection using A2 DNA produced a 264 scTCR/IgG1 molecule as determined using an ELISA assay with antibodies specific to the TCR and to the IgG1 isotype.

### Example 14. Demonstration of Anti-Tumor Effects of Modified TCR In Vitro

In example 11 we showed the TCR/IL-2 fusion protein able to mediate conjugation between a T cell and an antigen presenting cell pulsed with the correct peptide. Now we are interested in whether the crosslinking mediated by the fusion protein results in the destruction of the target cell. To determine if indeed this is the case, we will use an in vitro killing assay. Briefly, effector cells are generated from isolated murine splenocytes and cultured for three days at 370 C in 5% CO2 in the presence of stimulation with soluble recombinant human IL-2 (50 ng/ml) and anti-CD3-zeta mAb (145-2C11; 10 ng/ml). After three days in culture with stimulation, double staining of cells is carried out using anti-CD8 and anti-CD25 mAbs and flow cytometry. Detection of a double positive population is indicative of successful generation of effector CTL.

The killing assay is carried out using labeled target cells (e.g. peptide pulsed T2 cells, various carcinomas) with calcein-AM dye. Live cells (targets) incorporate the dye, are then washed and added to a 96 well plate containing effector cells and either the TCR/IL-2 fusion protein or positive or negative control proteins IL-2 and TCR-k fusion respectively. The ratios of effector to target cell will generally be 5:1, 10:1 and 20:1. The assay components are then incubated for 2 to 4 hours at 37° C. and the release of calcein-AM to the culture supernatant is measured. The specific release of calcein-AM is measured or compared to the non-specific control of spontaneous released calcein-AM.

### Example 15

### In Vivo Demonstration of Anti-Tumor Effects of Modified TCR.

In order to test the ability of the TCR/IL-2 fusion protein to facilitate elimination of human tumors that naturally express both A2 and p53, we will use a model in which such tumors are established in Nude and SCID mice. This model has also been used in Dr. Sherman's laboratory to test the efficacy of T cell clones and immunocytokines directed against human tumors. Tumors known to express p53 and to be specifically killed by p264 specific CTL will be tested for their ability to grow in Nude and SCID mice. Candidates include MDA-238, BT549, MCF-7, Caski (cervical carcinoma), and HepG2 cells. The cells (1×10⁶) will be implanted subcutaneously into Nude and SCID mice and allowed to establish for 7 days prior to treatment. Initially we will determine for each construct under evaluation, whether tumor growth is inhibited when mice receive the TCR-IL-2 fusion molecule alone. Although we anticipate that T cells will be required as effector cells for tumor elimination, other lymphoid cells present in the Nude and SCID due to leakiness may have effector function that can be triggered by the fusion protein, and this could inhibit tumor growth. This is particularly true in the presence of bispecific antibody molecules that have Fc or cytokines capable of stimulating nonlymphoid components of the innate immune system. Once we have determined the ability of the fusion protein to affect tumor growth, we will then test different T cell populations for effector function. The previous experiments in the murine tumor model will provide the information necessary to decide if naïve or activated T cells will be delivered. For control purposes, we will deliver activated T cells as effectors into mice without concurrent TCR-IL-2 fusion treatment.

### Example 16

### Preparation of Doxorubicin (Dox) Conjugates and In Vitro Characterization of Anti-Tumor Properties.

The purpose of this example is to develop an immunoconjugate using the 264 scTCR-C-kappa and a cytotoxic drug such as Doxorubicin. Dox, a member of the anthracycline family of drugs, is one of the most potent anti-cancer drugs known but its clinical application has been limited due to its cardio-toxicity. An attempt to overcome the cardio-toxicity has been to attach the Dox to a carrier molecule, such as an anti-tumor mAb, to deliver the drug specifically to tumor sites. Results from studies in pre-clinical models have demonstrated that Dox-immunoconjugates can kill tumor cells more effectively with less toxicity than equivalent doses of the free drug.

In this example, we will prepare and purify a 264 scTCR-Dox conjugate and then carry out several studies with the immunoconjugate to characterize its tumor killing activity in vitro and in vivo. First, the optimal number of Dox molecules coupled to the scTCR-k fusion protein will be determined. The amount of Dox internalized by a cell will directly influence the rate and efficiency of tumor killing. Therefore, if we assume the number of peptide/MHC targets displayed on the tumor cell is limiting, then coupling increasing numbers of Dox molecules onto the scTCR may be desirable. However, the stability of the linkage between the scTCR and the Dox group will have to be sufficiently high enough to prevent non-specific cell cytotoxicity in vivo associated with shedding of Dox. Collectively, findings from these studies may be used to predict the performance of the TCR-Dox conjugate in pre-clinical studies.

### Example 17

### Construction and Generation of Single-Chain TCR/IL-2 Fusion Proteins.

We have constructed a soluble TCR fusion protein comprising a three domain single-chain TCR linked to human IL-2 via a peptide linker. For the single-chain TCR (scTCR) fusion protein construct, DNA encoding the Va and Vβ/Cβ regions was generated either by RT-PCR from RNA isolated from a T cell clone or by PCR from an isolated TCR gene. For the 264scTCR, the TCR domain is specific to human p53 (aa 264-272) peptide presented in the context of HLA-A2.1. The TCR domains for the MART-1scTCR and CMVscTCR are specific to MART-1 (aa27-35) and CMV-pp65 (aa495-503), respectively, presented in the context of HLA-A2.1. The single-chain TCR gene encodes a protein wherein the carboxyl-terminal end of the variable region of the TCRα chain was fused via a flexible linker (G₄S)₄ (SEQ ID NO: 5) to the N-terminus of the Vβ to generate the antigen binding portion of the TCR. The Cβ domain, which is directly linked to the Vβ domain, was truncated at the amino acid residue just prior to the final cysteine, removing the transmembrane and cytoplasmic domains, to generate a soluble single-chain TCR molecule. The scTCR/IL-2 gene was created that encodes human IL-2 fused to the scTCR portion via a 19 amino acid short linker sequence. The scTCR/IL-2 gene was cloned with a mammalian cell expression vector where transcription is driven by a CMV promoter, secretion is directed by an antibody light chain leader sequence, and vector selection is carried out by G418 resistance. To generate the scTCR/IL-2 fusion protein, the scTCR/IL-2 expression vector was stably transfected into CHO-K1 cells. Stable transfectants secreting scTCR/IL-2 fusion protein were selected using ELISA assays with antibodies specific to the TCR and/or IL-2 domains. The scTCR/IL-2 fusion protein was purified from cell media by immunoaffinity chromatography with anti-TCR antibody conjugated to Sepharose. Based on SDS-PAGE analysis, the purified fusion protein exhibited the correct molecular weight of the intact scTCR/IL-2 molecule.

### Example 18

MHC/Peptide Binding Ability of the TCR Portion of the scTCR/IL-2 Fusion Protein

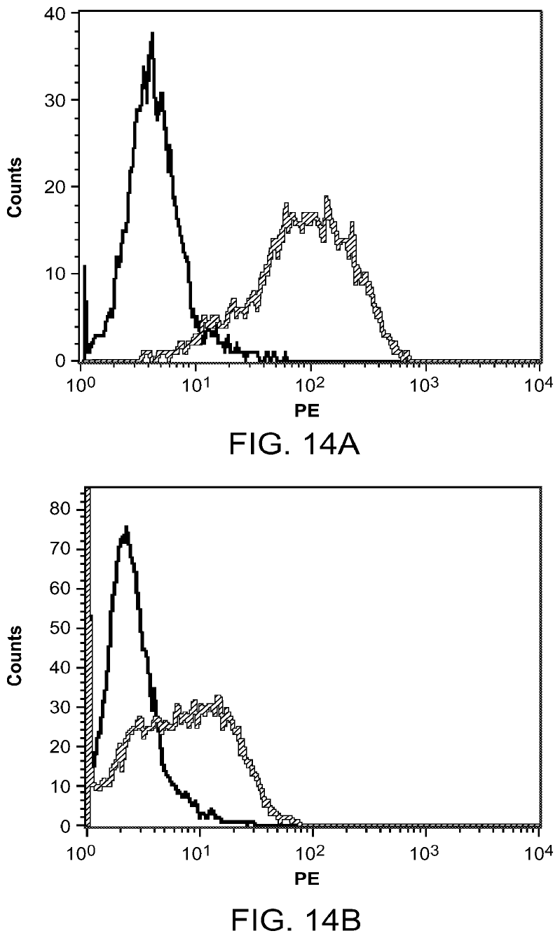

The ability of the 264scTCR/IL-2 fusion protein to bind to peptide loaded MHC was determined by flow cytometry. T2 cells were loaded with p53 (aa 264-272) or p53 (aa 149-157) (control) peptide and stained with 264scTCR/IL-2 fusion protein. Cells loaded with p53 (aa 264-272) stained positively with 264scTCR/IL-2 when detected with either the anti-TCR Cβ mAb or the anti-IL-2 detection antibody (FIGS. 14A and B). Cells loaded with p53 (aa 149-157) control peptide did not stain with 264scTCR/IL-2 plus anti-TCR Cβ mAb or anti-IL-2 detection antibodies. However, loading the T2 cells with either peptide stabilized HLA-A2 complexes on the cell surface as demonstrated by staining with BB7.2, an anti-HLA-A2 monoclonal antibody, suggesting that both the p53 (aa 149-157) and p53 (aa 264-272) peptides bind similarly to HLA-A2 complexes on these cells. These data indicate that the TCR portion of the 264scTCR/IL-2 fusion protein is capable of recognizing its specific peptide in the context of HLA-A2. Equivalent results demonstrating peptide specific staining were observed for the MART-1scTCR/IL-2 and CMVscTCR/IL-2 fusion proteins.

### Example 19

IL-2 Receptor Binding Ability of the IL-2 Portion of the scTCR/IL-2 Fusion Protein

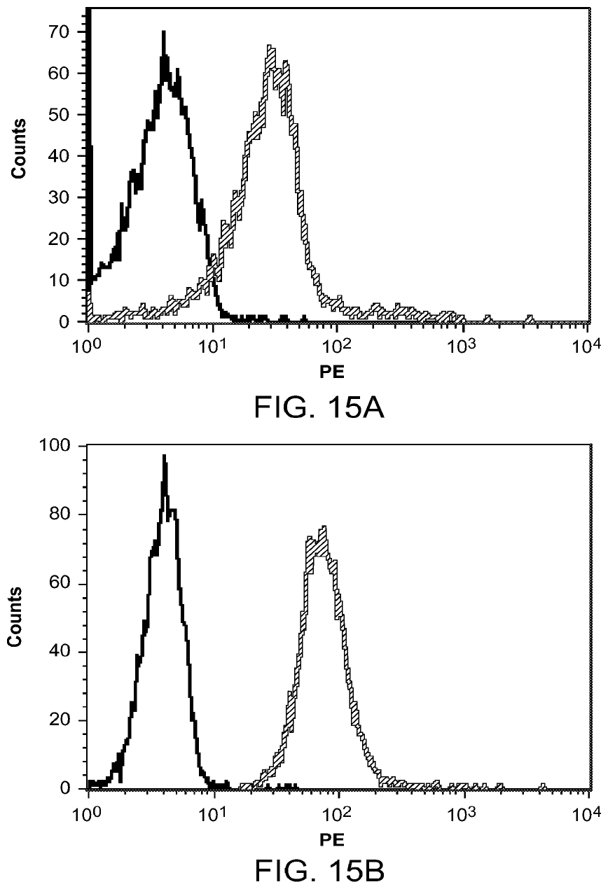

The IL-2 receptor (IL-2R) binding capability of the IL-2 portion of the 264scTCR/IL-2 fusion protein was studied by flow cytometry. Primary mouse splenocytes were isolated and stimulated with IL-2 and anti-CD3 to generate T cell blasts. Stimulated splenocytes that express IL-2R stained positively with 264scTCR/IL-2 fusion protein followed by detection of the 264scTCR domain with p53 (aa 264-272):HLA-A2 tetramers (FIG. 15A). Likewise, CTLL-2 mouse cytotoxic T lymphocytes, which constitutively express IL-2R, stained positively with the 264scTCR/IL-2 fusion protein but not with a 264scTCR/kappa fusion protein followed by p53 (aa 264-272):HLA-A2 tetramers (FIG. 15B). The lack of signal from the CTLL-2 cells incubated with a 264scTCR/mouse kappa chain fusion protein indicates that staining of these cells is mediated by the IL-2 portion of the 264scTCR/IL-2 fusion protein. These data demonstrate that the IL-2 portion of the 264scTCR/IL-2 fusion protein is capable of binding to the IL-2R and that the cell-bound TCR domain can be detected by peptide/HLA reagents. Thus, both scTCR and IL-2 domains of the fusion protein are active. Equivalent results demonstrating bifunctional binding activity were observed for the MART-1scTCR/IL-2 and CMVscTCR/IL-2 fusion proteins.

### Example 20

### Enhanced Binding of TCR/IL-2 Fusion Proteins to IL-2R Bearing Murine and Human Cells as Evidenced by an Extended Cell Surface Residency Time

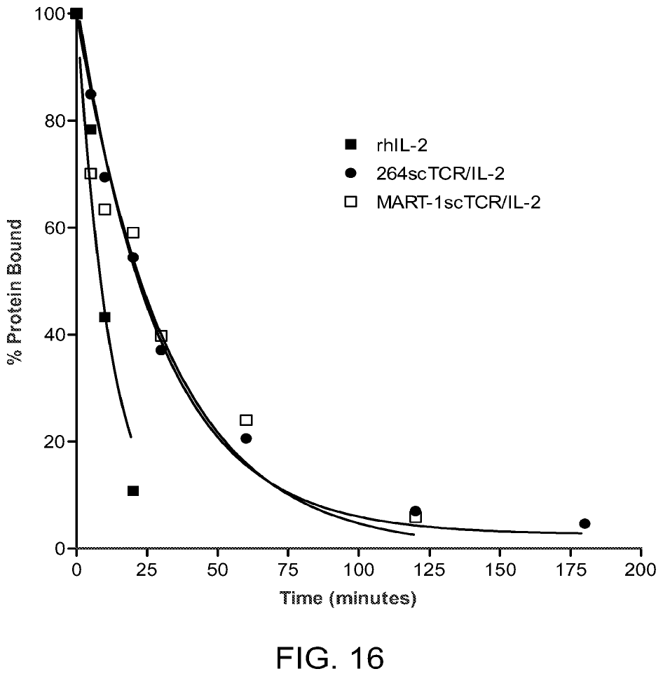

The cell surface residency time of the scTCR/IL-2 fusion protein on the IL-2R-bearing cells may influence the ability of the fusion protein to target or bridge effector cells with the TCR-specific tumor cells. To investigate this, binding of the scTCR/IL-2 fusion proteins and recombinant human IL-2 (rhIL-2) to IL-2R bearing CTLL-2 cells was directly compared by flow cytometry. FIG. 16 shows the results of such a study in which 264scTCR/IL-2, MART-1scTCR/IL-2 or rhIL-2-coated cells were incubated in media at 37° C. for up to 180 min and the level of proteins remaining on the cell surface was detected with anti-IL-2 mAb. As shown at the initial time point (t=0), addition of equivalent molar amounts of 264scTCR/IL-2, MART-1scTCR/IL-2 or rhIL-2 to CTLL-2 cells resulted in specific staining with PE-labeled anti-IL-2 mAb. Incubation at 37° C. resulted in cellular internalization and/or dissociation of the proteins as measured by a decrease in anti-IL-2 mAb binding. The cell surface half-life of rhIL-2 was determined to be 8.2 min by this method, a value similar to the 10 min t_{1/2} reported by Robb et al. 1987. J. Exp. Med. 165:1201. The calculated half-lives of 264scTCR/IL-2 and MART-1scTCR/IL-2 interaction with CTLL-2 IL-2R were approximately 20.3 and 22.7 min, respectively. Similar studies done with activated human PBMC indicated that 264scTCR/IL-2 specifically binds to the human IL-2R bearing cells with a half-life of about 20 min (data not shown), equivalent to the half-life seen for CTLL-2 cells.

These results indicate that the IL-2 domain of c264scTCR/IL-2 and MART-1scTCR/IL-2 showed more stable binding to IL-2R on cells than was observed for rhIL-2. This more stable interaction may allow the scTCR/IL-2 fusion protein to bind IL-2R-bearing immune cells and more effectively direct the scTCR/IL-2-coated cells against diseased cells expressing the peptide/HLA target.

### Example 21

Biological Activity of scTCR/IL-2 Fusion Protein In Vitro and In Vivo

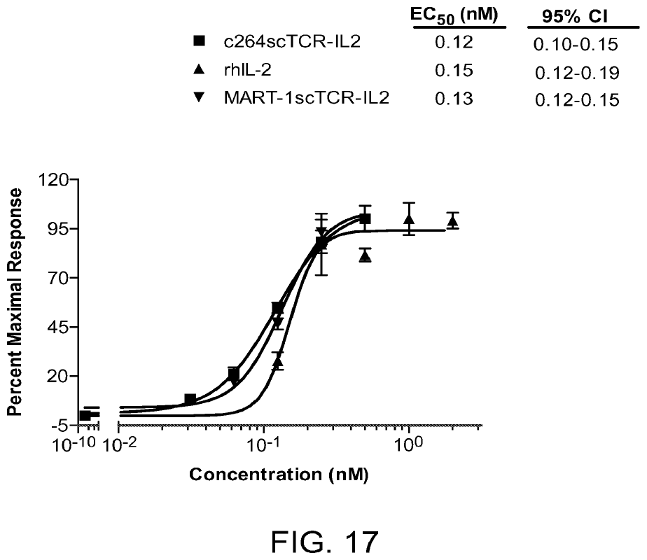

To demonstrate biological activity of the IL-2 portion of the scTCR/IL-2 fusion protein, IL-2 dependent CTLL-2 cells were cultured with either 264scTCR/IL-2, MART-1scTCR/IL-2 or rhIL-2 at various concentrations and cell viability was assessed using WST-1. For each protein, a dose-dependent response of CTLL-2 proliferation was observed wherein there was more cell proliferation at higher concentrations of protein. The protein concentration providing a half-maximal effect (EC50) was determined (FIG. 17). Given the four-fold difference in molecular weight between rhIL-2 and the scTCR/IL-2 fusion proteins, the three proteins exhibited similar activity on an equal molar basis (EC50: rhIL-2, 0.15 nM; 264scTCR/IL-2, 0.12 nM; and MART-1scTCR/IL-2, 0.13 nM), indicating that 264scTCR/IL-2 and MART-1scTCR/IL-2 fusion proteins have equivalent bioactivity as rhIL-2 for stimulating immune cell proliferation.

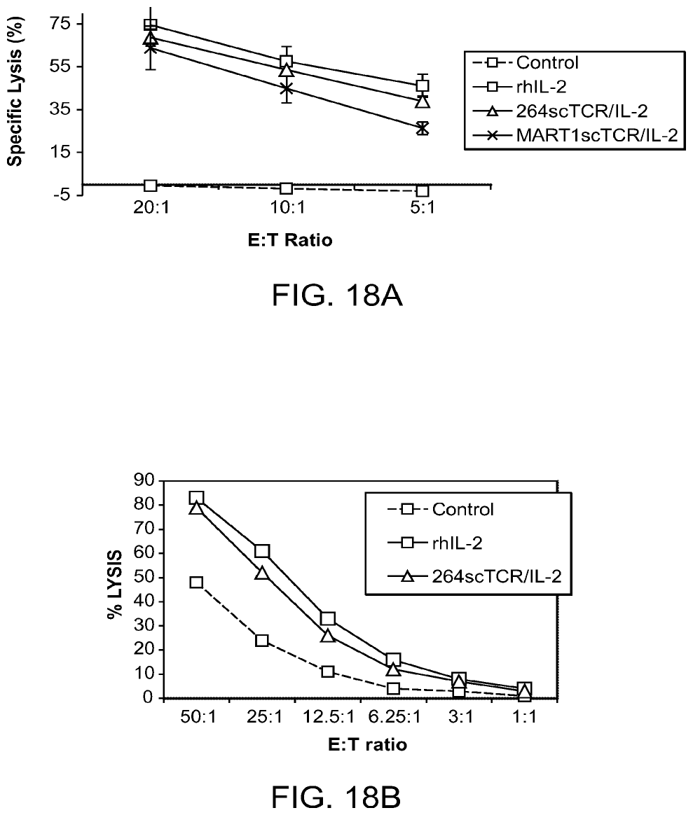

The capability of these proteins to stimulate immune effector cell responses was also examined. Athymic nude mouse splenocytes were incubated for four days in media containing equivalent molar amounts of 264scTCR/IL-2, MART-1scTCR/IL-2 or rhIL-2. Splenocytes were then mixed with NK-sensitive YAC-1 target cells at different ratios to determine cell-mediated cytotoxic activity. Equivalent levels of lytic activity was observed from the mouse splenocytes stimulated by 264scTCR/IL-2, MART-1scTCR/IL-2 or rhIL-2 (FIG. 18A). Short term stimulation of human PBMC or enriched human NK cells with 264scTCR/IL-2 or rhIL-2 also resulted in equivalent levels of cytotoxic activity (FIG. 18B). These results indicate that the scTCR/IL-2 fusion proteins have equivalent bioactivity as rhIL-2 for stimulating cytotoxic immune effector cell activity.

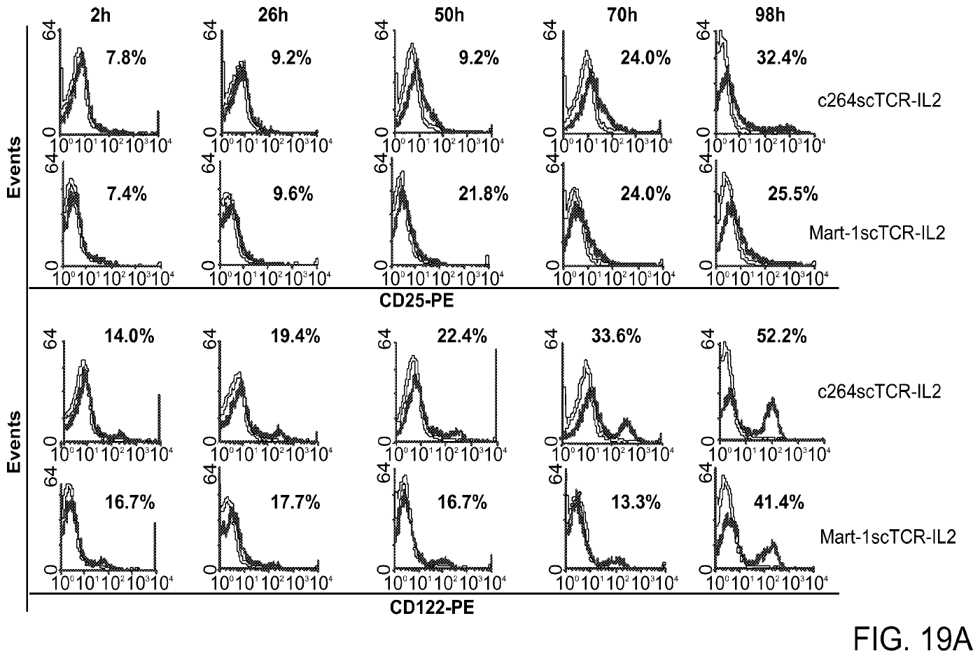

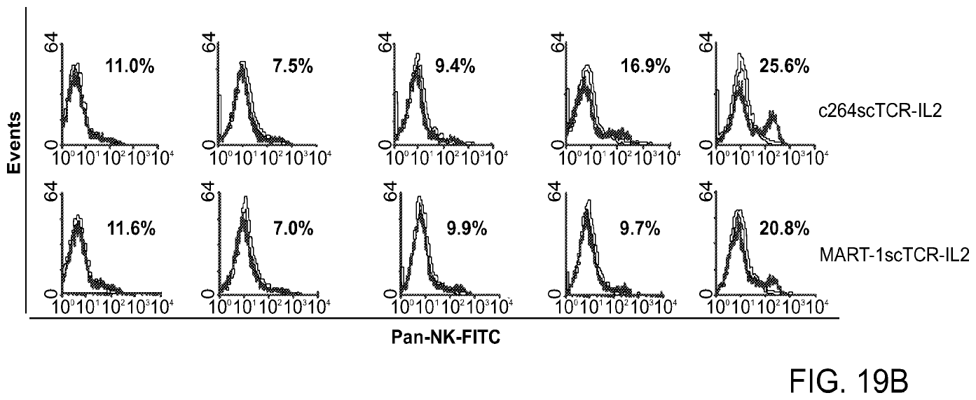

The effect of the scTCR/IL-2 fusion proteins on immune cells was also investigated in vivo. Repeated daily intravenous administration of 264scTCR/IL-2 at 32 μg/dose in A375 tumor-bearing nude mice resulted in an increase in serum IFN-γ levels and enlargement of lymph nodes and spleens, consistent with reported effects for rhIL-2 (Rosenstein et al., 1986. J. Immunol. 137:1735). Changes in IL-2R subunit expression were also assessed in immune cells isolated from the spleens, lymph nodes and bone marrow of 264scTCR/IL-2- or MART-1scTCR/IL-2-treated mice. Splenocytes expressing IL-2Rα (CD25) or IL-2≈ (CD122) increased approximately three-fold with repeated daily treatments of either scTCR/IL-2 fusion protein such that peak expression of the IL-2R subunits was observed one day after the fourth treatment (FIG. 19). In addition, repeated administration of 264scTCR/IL-2 or MART-1scTCR/IL-2 resulted in a treatment-dependent increase in NK cells with a similar time-course observed for the IL-2R markers. Elevated expression of CD122 and CD25 was also observed in cells isolated from the lymph nodes and bone marrow of mice following repeated daily 264scTCR/IL-2 treatment. These findings are also consistent with the known biological activities of IL-2 and suggest that repeated scTCR/IL-2 fusion protein administration augments further immune responsiveness to the fusion protein via IL-2R-positive cells (Puri et al. 1990 Cancer Res. 50:5543, Depper et al., 1985 PNAS 82:4230; Demaison, 1998 J. Immunol. 161:1977) Collectively, the results from these studies indicated that the 264scTCR/IL-2 and MART-1scTCR/IL-2 showed equivalent IL-2-like biologic activity in vitro and in vivo.

### Example 22

Conjugation of Cells Mediated by 264scTCR/IL-2 Fusion Protein

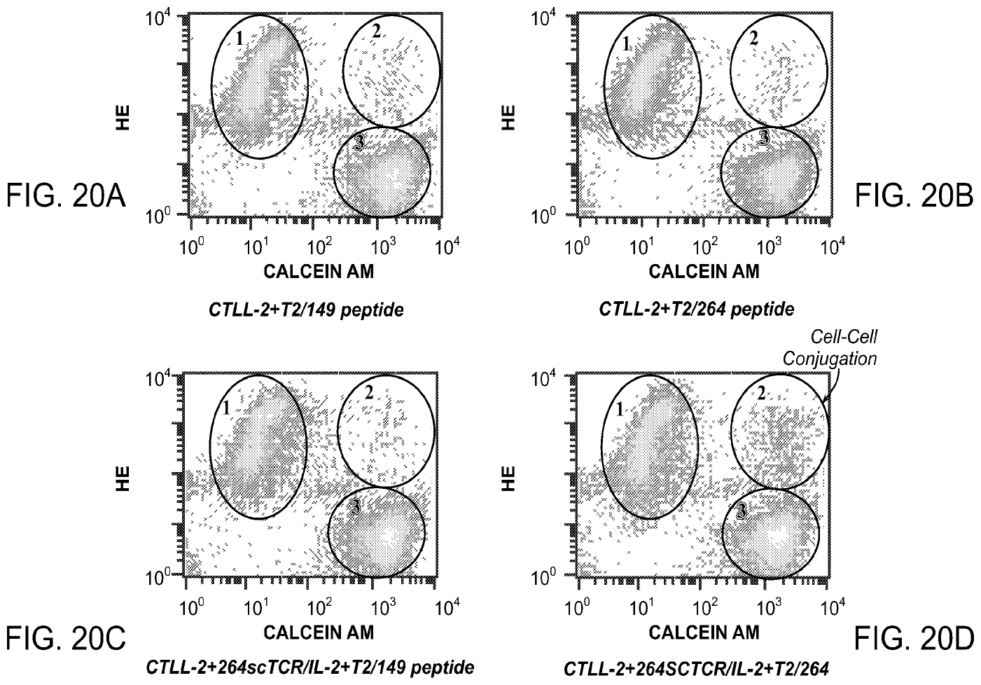

In order for the 264scTCR/IL-2 fusion protein to be useful as a therapeutic agent, it must be able to bring together target and effector cells through its TCR and cytokine portions, respectively. To demonstrate that the 264scTCR/IL-2 fusion protein can effectively conjugate cells, T2 cells were loaded with either p53 (aa 264-272) or p53 (aa 149-157) peptides and then labeled with dihydroethidium (HE). CTLL-2 cells were labeled with calcein AM and the two labeled cell populations were mixed and incubated in the presence or absence of 264scTCR/IL-2 fusion protein. Samples were analyzed by flow cytometry. When the two cell populations were incubated in the absence of the 264scTCR/IL-2 fusion protein or when the T2 cells were loaded with control peptide and incubated with the CTLL-2 cells in the presence of 264scTCR/IL-2 fusion protein, the cells remained as two distinct populations on the flow cytometry histograms representing approximately 45% of the total population each (FIG. 20, regions 1 and 3) with only approximately 0.9% of the total population falling in the double stained cell window (FIG. 20 region 2). However, when the T2 cells were loaded with p53 (aa 264-272) peptide and incubated with the CTLL-2 cells in the presence of the 264scTCR/IL-2 fusion protein, a double staining population of cells appears, representing 3.25% of the total population (FIG. 20 region 2, conjugated cells), demonstrating that p53 (aa 264-272)/HLA-A2-positive T2 cells were conjugated to CTLL-2 cells via the 264scTCR/IL-2 fusion protein. Thus, the scTCR/IL-2 fusion protein could be useful in a therapeutic approach for bringing together immune effector cells and tumor cells and stimulating immune cell-mediated activity against the tumor.

### Example 23

Pharmacokinetic Profile of 264scTCR/IL-2 Fusion Proteins in Mice and Humans

The pharmacokinetic parameters of 264scTCR/IL-2, MART-1scTCR/IL-2 and rhIL-2 were evaluated in the HLA-A2.1/K^{b}-transgenic mouse strain. The presence of the HLA-A2.1 domain, for which c264scTCR/IL-2 is restricted, may influence the pharmacokinetics of this fusion protein and should give a more relevant “humanized” view of the pharmacokinetics than other mouse strains. Mice were injected intravenous with 1.6 mg/kg 264scTCR/IL-2 or MART-1scTCR/IL-2 or 0.4 mg/kg (molar equivalent dose) rhIL-2 and blood was collected at various time points from 5 minutes to two weeks post injection. Serum concentrations of 264scTCR/IL-2 and MART-1scTCR/IL-2 were evaluated using two ELISA formats, one (anti-IL-2 Ab) which detects the intact bi-functional protein and the other (W4F Ab) which detects only the TCR domain. Concentrations of rhIL-2 were detected with a standard IL-2-specific ELISA.

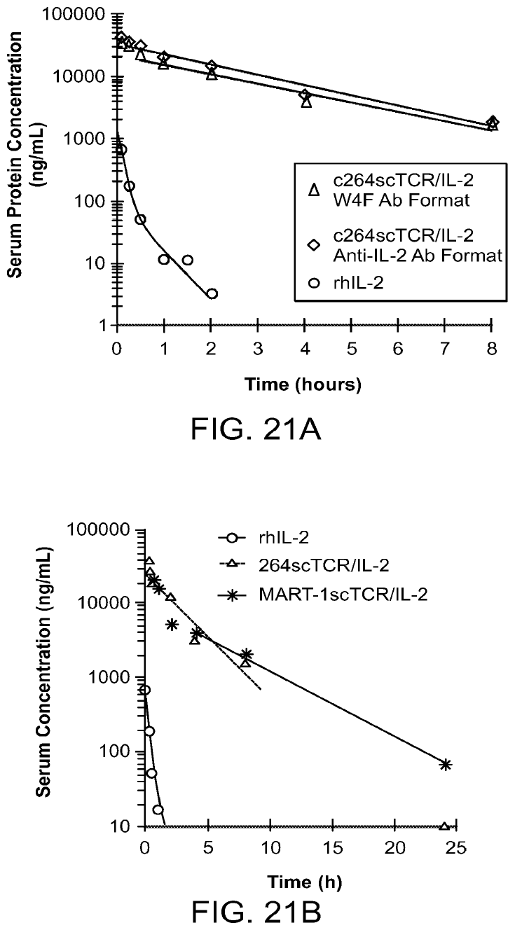

The predicted fit and actual data for 264scTCR/IL-2 and rhIL-2 serum concentrations following the single intravenous bolus injection are shown in FIG. 21A. The estimated half-life of 264scTCR/IL-2 using anti-IL2 Ab or W4F Ab ELISAs was 1.82 hrs or 2.00 hrs, respectively. These results indicate that the bi-functional fusion protein was not cleaved apart in vivo. The clearance (CI) of 264scTCR/IL-2 ranged from 0.376 to 0.493 and the volume of distribution at steady state (Vss) from 0.96 to 1.33 depending on the assay format. The average Vss is about 16% larger than the plasma volume suggesting modest distribution of 264scTCR/IL-2 into tissues. In comparison, rhIL-2 had an absorption half-life (t_{1/2}-abs) of ˜0.07 hrs and a terminal half-life of 0.39 hrs. The clearance of rhIL-2 was 42.0 ml/hr and the volume of distribution at steady state was 9.93 ml. These results indicate that 264scTCR/IL-2 fusion protein displays a five-fold longer terminal half-life and is cleared >90-fold slower than rhIL-2.

The results comparing the pharmacokinetics of 264scTCR/IL-2, MART-1scTCR/IL-2 and rhIL-2 are shown in FIG. 21B. In this study the terminal half-lives of rhIL-2, 264scTCR/IL-2 and MART-1scTCR/IL-2 were determined to be about 0.33, 1.84 and 3.50 h, respectively. Thus, MART-1scTCR/IL-2 has a serum half-life about 11 times longer than rhIL-2. Consistent with the results observed for 264scTCR/IL-2, pharmacokinetics of MART-1scTCR/IL-2 were equivalent whether assessed by the anti-IL-2 or W4F Ab-based ELISAs, indicating that the bi-functional fusion protein was not cleaved apart in vivo.

The results observed in HLA-A2 transgenic mice are borne out by pharmacokinetic analysis for cancer patients treated with fusion protein. The terminal half-life of 264scTCR/IL-2 following a single bolus intravenous infusion (0.015 mg/kg) averaged 4.9 hours and over 75% of the administered dose was detectable in the serum. Again analysis with different ELISA formats verified that the scTCR/IL-2 fusion protein remained intact in the patient's serum. As reported in the product label for rhIL-2 (Proleukin®), studies in humans indicated that upon completion of intravenous infusion, only 30% of the rhIL-2 dose is measurable in the serum. These results are consistent with the very rapid (<1 min) uptake of the majority of rhIL-2 in the lungs, liver, kidney and spleen observed in animal studies. Of the drug remaining in the circulation, the distribution and elimination t_{1/2} for rhIL-2 was 13 and 85 minutes, respectively (Proleukin® Drug Label). Therefore, based on these findings and the results of the animal and human studies, the scTCR/IL-2 fusion protein has a longer serum half-live and higher serum recovery than rhIL-2. Therapeutic use of rhIL-2 is limited by its very short half life such that patient receiving this drug need to be infused every 8 hours during the FDA-approved 5 day treatment cycle. The longer half-life, modest tissue distribution, slow clearance and stable bifunctionality of the scTCR/IL-2 fusion proteins provide significantly more favorable pharmacokinetic properties than would be predicted for IL-2-based therapeutic agents.

### Example 24

### Single-Chain TCR/IL-2 Fusion Proteins Exhibit Anti-Tumor Efficacy In Vivo

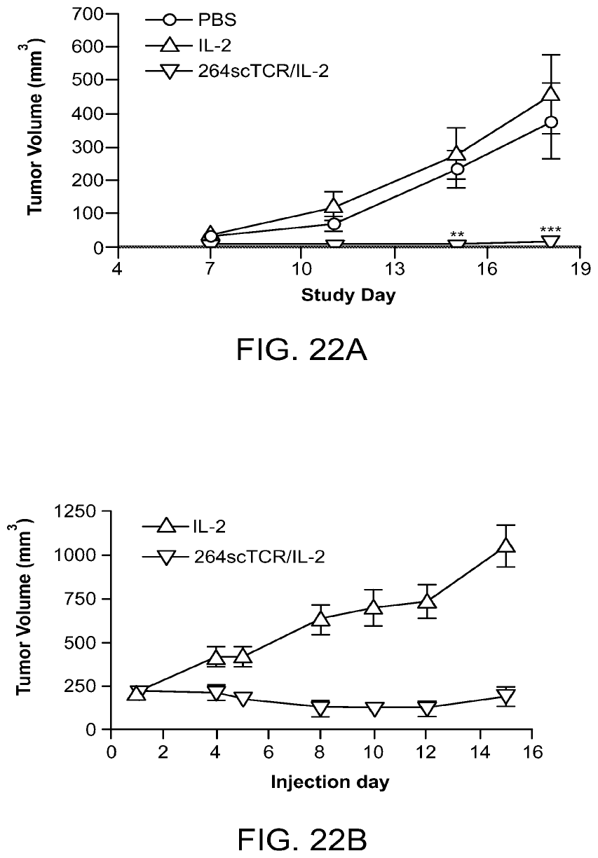

To determine the therapeutic effects of the scTCR/IL-2 fusion proteins compared to rhIL-2, we examined antitumor activity in a primary tumor growth model with the human A375 cell line in nude mice. Tumor cells were injected subcutaneously into nude mice and tumors were allowed to establish for three days before treatment began. Tumor-bearing mice were injected intravenously with 264scTCR/IL-2 (1.6 mg/kg), the molar equivalent of rhIL-2 (0.4 mg/kg), or the dose volume equivalent of PBS daily for four days, followed by treatment every other day for a total of 9 doses. During the study, tumor growth was measured and the tumor volumes were plotted (FIG. 22A). As expected, all mice treated with PBS developed tumors that grew to a large size. All of the mice treated with rhIL-2 developed tumors, while only 50% of mice treated with 264scTCR/IL-2 developed tumors and these remained small throughout the study (FIG. 22A). We also examined the effects of 264scTCR/IL-2 on large well-established A375 melanoma tumors. In this study, A375 cells were injected subcutaneously into nude mice and the tumors were allowed to establish and grow to either 75 mm³ (not shown) or 200 mm³ (FIG. 22B). The mice were then injected intravenously following the same time course as described above with 264scTCR/IL-2 or rhIL-2. Again, treatment with 264scTCR/IL-2 led to marked inhibition of tumor growth and partial to complete regression of tumors in most mice by the completion of the dosing regimen, while tumors in the rhIL-2 group continued to grow at a rapid rate increasing over 4 fold during the course of treatment (FIG. 22B). These results demonstrate that scTCR/IL-2 fusion protein has significantly greater efficacy against well-established human xenograft tumors than rhIL-2 alone.

### Example 25

Dose Dependence of Antitumor Activity of 264scTCR/IL-2 Against A375 Tumors is Dose Dependent.

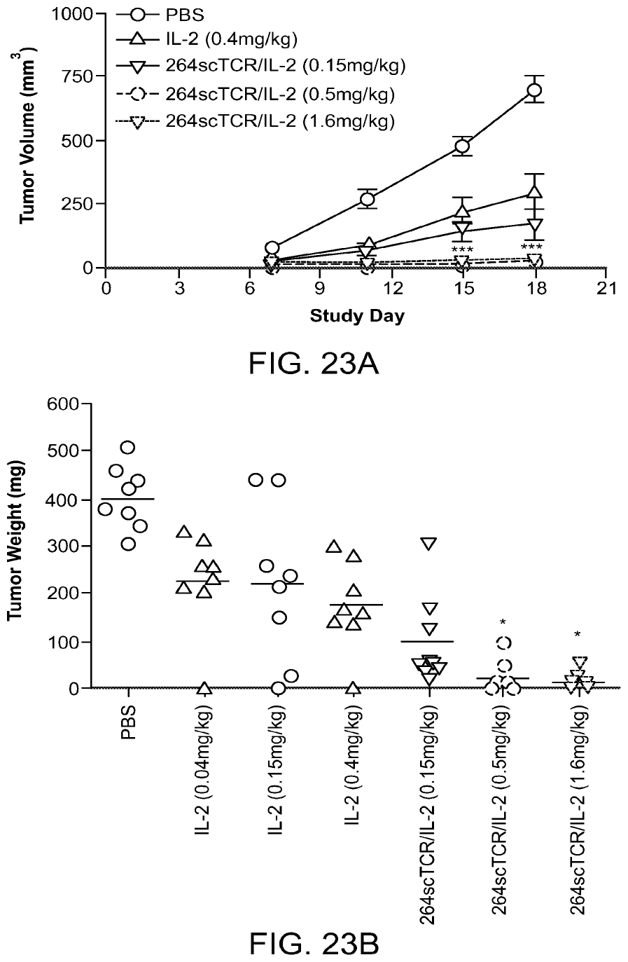

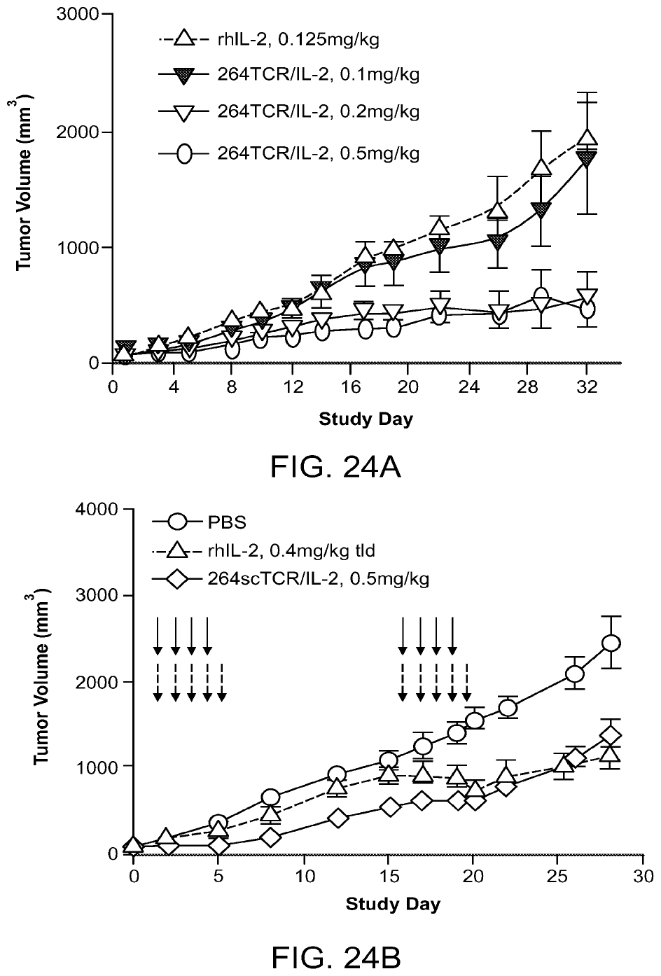

An additional study was performed to determine whether the antitumor activity of 264scTCR/IL-2 against A375 tumors is dose dependent. In this study, A375 cells were injected subcutaneously into nude mice and the tumors were allowed to establish for three days. Mice were then injected intravenously with 264scTCR/IL-2 (1.6, 0.5 or 0.15 mg/kg/dose), rhIL-2 (0.4 mg/kg/dose), or the dose volume equivalent of PBS for a total of 9 doses as described above. Only four of the eight mice treated with the 1.6 mg/kg of 264scTCR/IL-2 developed tumors and these remained small throughout the study. At the 0.5 mg/kg dose of 264scTCR/IL-2, six of the eight mice developed tumors, which again remained small throughout the study. With the 3 μg dose of 264scTCR/IL-2, all of the mice developed tumors but the average tumor size was significantly smaller than that of the PBS treated mice (FIG. 23A). Moreover, direct comparison of tumor weight at the end of the study showed that both the 1.6 mg/kg and 0.5 mg/kg doses of 264scTCR/IL-2 were significantly more effective at reducing tumor burden than the high dose of rhIL-2 (FIG. 23B). In addition, when 264scTCR/IL-2 or rhIL-2 were administered to tumor-bearing nude mice with a daily treatment schedule more similar to Proleukin® therapy (i.e., four daily injections followed by a 10-day rest period and then four more daily injections), 264scTCR/IL-2 at 0.5 or 0.2 mg/kg significantly inhibited tumor growth when compared with 0.125 mg/kg rhIL-2 (FIG. 24A). This treatment schedule also resulted in more effective antitumor activity than was observed in the 9-dose schedule and was selected as an initial dosing regimen for clinical development. To further compare 264scTCR/IL-2 treatment with that of Proleukin®-based thrice-daily high dose rhIL-2 administration, tumor-bearing nude mice treated with 0.5 mg/kg 264scTCR/IL-2 or PBS daily for four injections followed by a 10-day rest period and then four more daily injections or with 0.4 mg/kg rhIL-2 every 8 hrs for five days followed by a 9-day rest period and then every 8 hours for five more daily injections. In this study, each single dose of rhIL-2 had 3.2 times the activity as the 264scTCR/IL-2 dose and the cumulative dose of rhIL-2 was 12 fold higher than that of 264scTCR/IL-2. As shown in FIG. 11B, both 264scTCR/IL-2 and rhIL-2 treatment inhibited tumor growth to a similar degree following two dosing cycles. However, the rhIL-2 regimen also exhibited more toxicity based on clinical observations (hypoactivity, mottled skin, hunched postures) and decreased body weight than was observed in 264scTCR/IL-2 treated mice. Given that 264scTCR/IL-2 was as effective as a 12-fold higher cumulative dose of rhIL-2 in reducing tumor burden without the overt signs of toxicity, this study provides additional support that 264scTCR/IL-2 offers safer and more effective treatment for cancer than current high dose rhIL-2 therapies.

### Example 26

Pharmacological Activity of the scTCR/IL-2 Fusion Protein

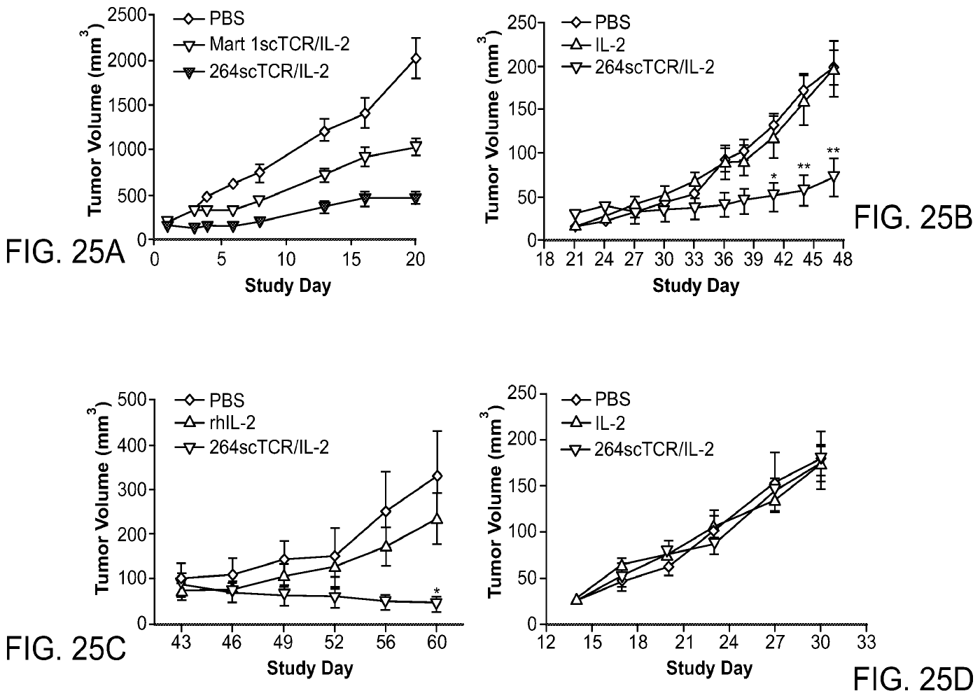

To better understand the pharmacological activity of the scTCR/IL-2 fusion protein, A375 tumor-bearing mice were treated with 264scTCR/IL-2 or a control scTCR/IL-2 fusion, MART1scTCR/IL-2, that does not recognize A375 tumor cells. In each case the treatment consisted of four daily i.v. injections followed by a 10-day rest period and then four more daily injections at 0.5 mg/kg scTCR/IL-2 fusion protein or an equivalent volume of PBS. The results shown in FIG. 25A indicate that 264scTCR/IL-2 treatment exhibits potent antitumor activity compared to PBS, whereas MART1scTCR/IL-2 provides intermediate but significant inhibition of A375 tumor growth in this model. In separate studies (data not shown), MART1scTCR/IL-2 was found to display equivalent IL-2 bioactivity as 264scTCR/IL-2 and a serum half-life of 3.2-3.6 hours in HLA-A2 transgenic mice, about twice that of 264scTCR/IL-2 and 10-fold that of rhIL-2. Thus, these results suggest that increasing the biological half-life of IL-2 without specific tumor binding, as is the case for the MART1scTCR/IL-2 fusion, leads to better antitumor activity than is seen with rhIL-2 alone in these models. However, the combination of improved IL-2 serum half-life and tumor recognition activity of the TCR domain creates an even more potent anti-tumor agent as exemplified by the 264scTCR/IL-2 fusion protein.

To further investigate whether the antitumor activity of 264scTCR/IL-2 is p53+/HLA-A2+ dependent, additional xenograft tumor studies were carried out using human tumors that vary in their expression of p53 and HLA-A2.1. The tumor lines used in this study were MBA-MD-231 (p53+/HLA-A2.1+), PANC1 (p53+/HLA-A2.1+), AsPC1(p53-/HLA-A2.1-) and HT29 (p53+/HLA-A2.1-). The expression level of p53, HLA-A2.1, as well as p53(aa264-272)/HLA-A2.1 complex displayed by these tumor lines was determined using our scTCR multimer technology in combination with antibodies as described in Zhu et al. 2006. J. Immunol. 176:3223. Primary tumors derived from these cells were established in nude mice following subcutaneous injection. Mice were then treated as previously described with 264scTCR/IL-2, rhIL-2 or PBS and tumor volumes were measured. As shown in FIG. 25B, rhIL-2 treatment had no effect on MBA-MD-231 tumor growth as compared to PBS while treatment with 264scTCR/IL-2 had a significant antitumor effect. Similar results showing significant antitumor effects of 264scTCR/IL-2 were observed in tumors derived from PANC1 cell line (FIG. 25C). In contrast, when 264scTCR/IL-2 was used to treat mice with established AsPC1 (FIG. 25D) or HT29 (not shown) tumors, tumor outgrowth was not inhibited and tumor size did not vary significantly between treatment groups. This lack of 264scTCR/IL-2 antitumor activity on tumors that do not express HLA-A2.1 further indicates that tumor targeting to the p53(aa264-272)/HLA-A2 complex is a substantial component of the efficacy observed when treating human xenograft tumors with 264scTCR/IL-2.

### Example 27

Single-Chain TCR/IL-2 Fusion Protein Directs Immune Cell Infiltration into Tumors

IL-2 has been shown to have stimulatory effects on a broad range of immune cell types including T cells, B cells, monocytes, macrophages, LAK cells and NK cells (15, 16). The primary tumors studies were carried out in mice lacking T cells, thus the anti-tumor effects of the 264scTCR/IL-2 fusion are likely due to cells of the innate immune system. To examine this in more detail, we stained A375 (p53+/HLA.A2.1+) and AsPC1 (p53-/HLA-A2.1-) tumor sections from 264scTCR/IL-2 and rhIL-2 treated mice with antibodies specific to various murine immune cell markers. While no differences were observed in A375 tumors stained with antibodies to Mac-3 (macrophage marker) or Ly-6 (neutrophil marker) (data not shown), CD45R-positive cells (i.e., NK cells and immature B cells in nude mice) observed in tumors of 264scTCR/IL-2 treated mice showed distinct differences in distribution and morphology compared to CD45R-positive cells (FIG. 26). Complete tumor infiltration of large CD45R+ cells was observed in 264scTCR/IL-2 treated A375 tumors, whereas in rhIL-2 treated A375 tumors only small CD45R+ cells were observed and they were primarily restricted to mouse tissue surrounding the tumor. Further, histological examination of the 264scTCR/IL-2 treated mouse tumor sections confirmed that B cells were not present, suggesting that the infiltration was due primarily to NK cell activation at the site of tumor burden. Conversely, in the AsPC1 tumors, which do not express HLA-A2.1 and did not respond to 264scTCR/IL-2 therapy, no differences were observed between treatment groups, and the cellular profiles resembled those seen for A375 tumors treated with rhIL-2 (FIG. 26). These data indicate that treatment with the scTCR/IL-2 fusion protein leads to NK cell activation and localization and penetration of these cells into tumors displaying the scTCR target. These effects were not observed following rhIL-2 administration.

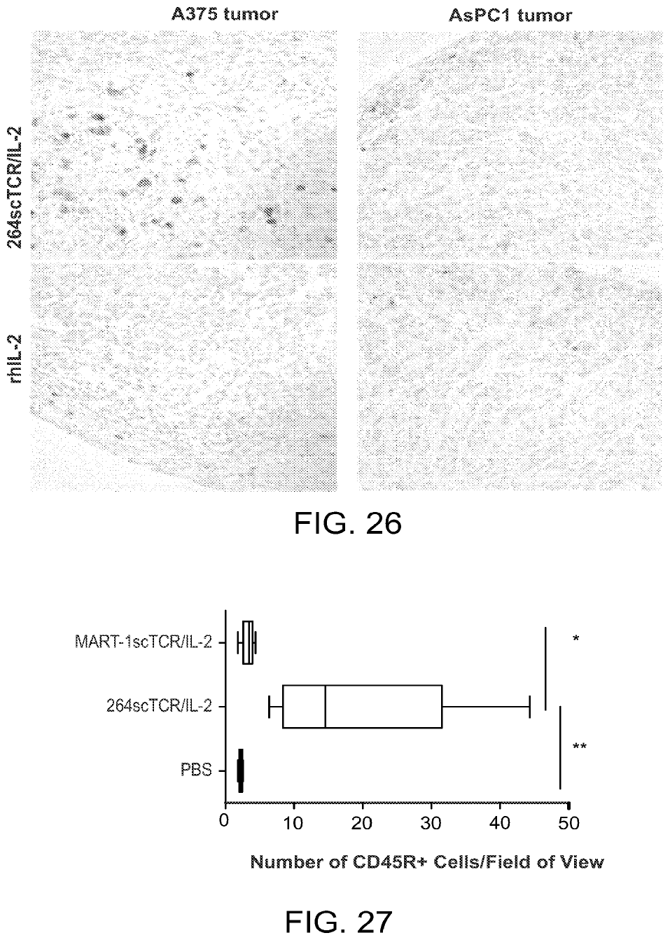

In a subsequent study, tumor infiltration of CD45R+ cells was compared in A375 tumor-bearing nude mice treated with 264scTCR/IL-2 (10 μg/dose), control MART-1scTCR/IL-2 (10 μg/dose) or PBS. Following four days of daily treatment, significantly more CD45R+ cell infiltration was observed in tumors of 264scTCR/IL-2 treated mice (14.5 CD45R+ cells/field of view) compared with that seen in tumors from mice treated with either MART-1scTCR/IL-2 (4.0 CD45R+ cells/field of view) or PBS (2.5 CD45R+ cells/field of view) (FIG. 27). These findings suggest that administration of the tumor-specific 264scTCR/IL-2 fusion protein first leads to the activation of immune cells and then the infiltration of CD45R+ effector cells into p53+/HLA-A2+ tumors. It should be noted that CD45R+ cell infiltration in the tumors also correlates well with the timing and relative levels of antitumor activity for the 264scTCR/IL-2 fusion protein.

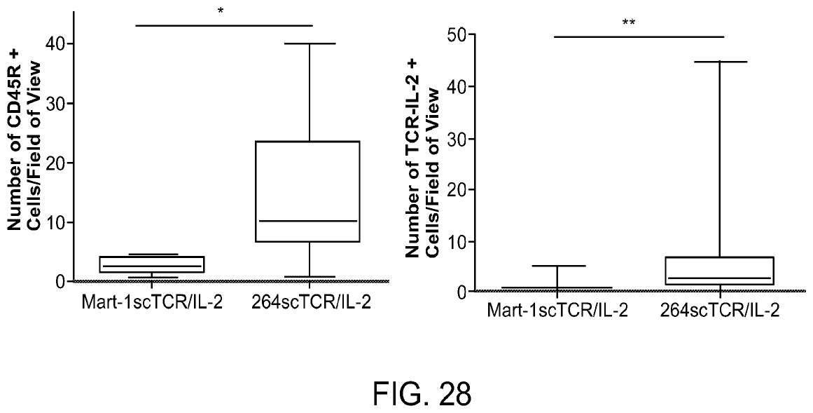

We used an adoptive cell transfer approach to directly demonstrate that the bifunctional 264scTCR/IL-2 molecules have the capability to bind and then retarget immune cells to antigen-positive tumors via the scTCR domain. Nude mouse splenocytes were activated in vivo by treating mice with 264scTCR/IL-2 or MART-1scTCR/IL-2 at 32 μg/dose daily for 4 days. The activated splenocytes were isolated and incubated with the respective scTCR/IL-2 fusion proteins in vitro to coat the surface of the cells prior to transfer into A375 tumor bearing nude mouse recipients. Tumors were then isolated 24 hrs later and levels of CD45R+NK cells were assessed. Significantly more infiltration of CD45R+ cells was observed in tumors from nude mice receiving splenocytes incubated with 264scTCR/IL-2 (10.3 CD45R+ cells/field of view) than in tumors from mice treated with splenocytes preincubated with the control MART-1scTCR/IL-2 (2.7 CD45R+ cells/field of view) (FIG. 28). In addition, when these sections were stained for either 264scTCR/IL-2 or MART-1scTCR/IL-2 fusion proteins, significantly more cells bound with detectable levels of 264scTCR/IL-2 were observed infiltrating into the tumors (264scTCR/IL-2: 3.0 BF1+ cells/field of view; MART-1scTCR/IL-2; 0.2 BF1+ cells/field of view) (FIG. 28). These data strongly support the notion that activated NK cells bound with 264scTCR/IL-2 molecules are retargeted to the tumor via the interaction between the scTCR domain of the fusion protein and the p53 peptide/HLA-A2 complexes at the tumor site. Moreover, these results bring to attention the potential use of scTCR/IL-2 fusion molecules in adoptive cell transfer strategies to provide immune effector cells with novel pre-selected tumor antigen binding capability.

### Example 28

In Vitro Treatment of Immune Cells with TCR/IL2 Fusion Protein Followed by Adoptive Cell Transfer Provide Improved Survival in Xenograft Tumor Animal Model

To demonstrate the anti-tumor efficacy of enriched allogenic mouse NK cells preincubated with ALT-801 (c264scTCR-IL2) on tumor growth, the following study was carried out using human NSCLC A549A2 tumor cells in an experimental metastasis model in nude mice.

Athymic nude mice (n=4 per group, female, 5-6 week old) were intravenously (IV) injected through the lateral tail vein with the human NSCLC tumor cell line A549-A2 at 5×10⁶ cells/mouse. The A549-A2 cell line represents a transfectant of the p53-positive A549 parental line carrying a vector expressing human HLA-A2.1 cDNA.

Spleens from A2 mice (B6 background) were collected and NK cells were isolated using a NK cell isolation kit from Miltenyi Biotech, Inc. according to the manufacturer's instruction. Briefly, a single cell suspension of splenocytes was prepared by homogenizing the spleens through a metal screen (60 mesh) in HBSS. Red blood cells were lysed in ACK red blood lysing buffer. Cells will be incubated with biotin-antibody cocktail (10 μL for 10⁷ cells) for 10 min at 4-8 C. The number of leukocytes was determined and 30 μL of buffer (PBS pH 7.2, 0.5% BSA and 2 mM EDTA) and 20 μL of anti-biotin MicroBeads per 10⁷ cells was added and the mixture was incubated at 4-8 C for 15 min. The cells were washed in 2 mL buffer and centrifuge at 300×g for 10 min. The cells were resuspended in 500 μL of buffer for loading to the MACS column. The flow through was collected and the purity of the NK cells was determined using FACScan analysis.

In order to activate the cells, NK cells (5×10⁶) were cultured at 37 C overnight in the presence or absence of TCR-IL2 fusion protein or rhIL-2 in T25 flasks in 10 ml RPMI1640 supplemented with 10% FBS. TCR-IL2 fusion protein was added at a concentration of 0.8 μg/mL and rhIL-2 was added at 0.2 μg/mL. These concentrations are equivalent to about 2600 IU/mL of IL-2 bioactivity. After overnight incubation, cells were harvested and preincubated in 0.5 mg/mL TCR-IL2 fusion protein or 0.125 mg/mL rhIL-2 in 100 μL on ice for 30 min. After wash in PBS (1 mL), cells were resuspended in PBS at 10×10⁶/mL for adoptive transfer.

On day 1, mice were injected i.v. via the tail vein with A549A2 tumor cells (5×10⁶) to establish pulmonary tumors. Fourteen days post tumor cell injection, mice were randomized and divided into 5 groups (n=4). Mice were treated with cyclophosphamide (CTX) via intraperitoneal injection at a dose of 200 mg/kg on days 14 and 21. NK cells (1×10⁶/mouse) preincubated with different TCR-IL2 fusion proteins or rhIL-2 were injected i.v. on days 16 and 22, and mice receiving PBS served as controls. A summary of the treatment schedule is a follows:

| | CTX | | NK cells | |
|---|---|---|---|---|
| | Dose | Injection | Dose | Injection |
| Group | (mg/kg) | (ip) | (×10⁶) | (iv) |
| CTX | 200 | Days 14, | 0 | Days 16, |
| | | 21 | | 22 |
| CTX+ NK/rhIL2 | 200 | Days 14, | 1 | Days 16, |
| | | 21 | | 22 |
| CTX + NK/MART- | 200 | Days 14, | 1 | Days 16, |
| 1scTCR-IL2 | | 21 | | 22 |
| CTX + NK/c264scTCR- | 200 | Days 14, | 1 | Days 16, |
| IL2 | | 21 | | 22 |

was monitored every day. Mice that became moribund were sacrificed and counted as dead. Mice surviving longer than 100 days post-tumor injection were considered as cured.

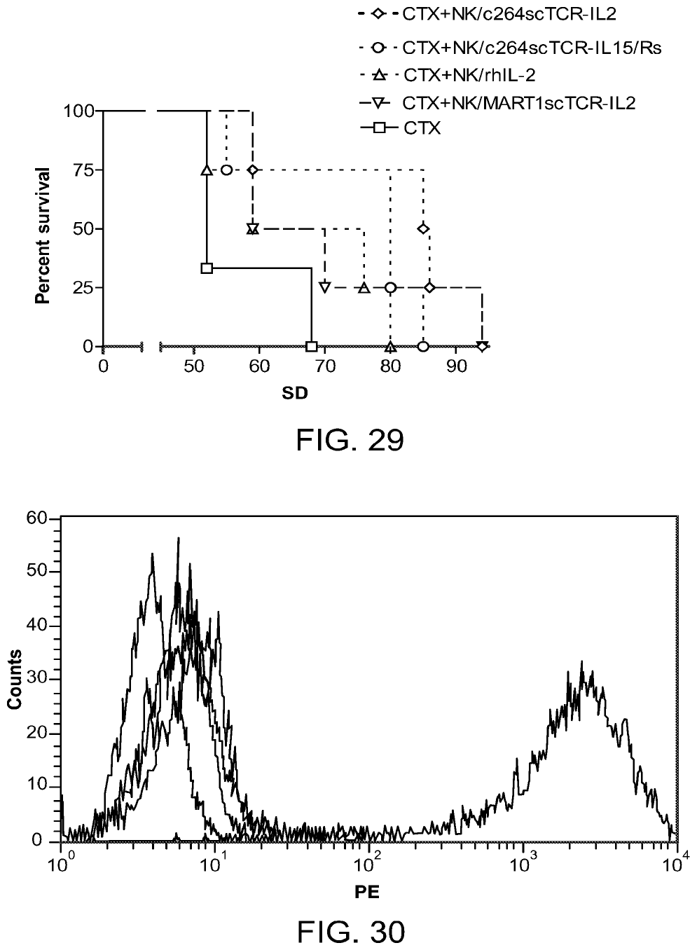

Median survivals for mice in the CTX, CTX+NK/rhIL-2, CTX+NK/MART1scTCR-IL2, and CTX+NK/c264scTCR-IL2 treatment groups are 52, 67.5, 64.5 and 85.5, respectively (FIG. 29). Thus, adoptive transfer of c264scTCR-IL2-activated NK cells resulted the longest median survival of 85.5 days whereas treatment with NK cells activated by the non-targeted MARTscTCR-IL2 or rhIL-2 resulted in a shorter and equivalent survival time. The results from this pilot experiment indicate that activation and targeting mouse NK cells with c264scTCR-IL2 may provide better antitumor activity than use of rhIL-2 or a control TCR-IL2 fusion.

### Example 29

### Construction and Characterization of Other Single-Chain TCR/Cytokine and Single-Chain TCR/Growth Factor Fusion Proteins

### Generation of Expression Vector Constructs

Fusion proteins were also constructed comprising a three domain TCR fused to either human or mouse IFNα, or human or mouse GM-CSF. For example. TCR fusion protein constructs were generated comprising the Vα and Vβ/Cβ regions from the HLA-A2 restricted TCR specific for the unmutated human p53 (aa 264-272) peptide described above. The carboxyl-terminal end of the variable region of the TCRα chain (Vαβ) was fused via a flexible linker (G₄S)₄ (SEQ ID NO: 5) to the N-terminus of the Vβ to generate the antigen binding portion of the TCR. The Cβ domain, which is directly linked to the Vβ domain, was truncated removing the transmembrane and cytoplasmic domains, to generate a soluble single chain TCR molecule. Alternatively a single-chain LCMV-CR construct was generated from the murine P14 (specific for the lymphocytic choriomeningitis virus glycoprotein peptide, gp33-41) TCR following PCR amplification of the Vα and Vβ-Cβ genes from nucleic acid materials obtained from the p14 TCR transgenic mouse. For the GM-CSF and IFNα fusion proteins, the previously defined coding regions for the mature mouse and human GM-CSF or IFNαb proteins were PCR amplified from nucleic acid materials obtained from the appropriate mouse and human immune cells. These coding regions were used to replace the IL-2 regions in the 264scTCR-IL2 fusion construct described above or as a similar construct containing the LCVMscTCR gene. All fusion protein constructs were confirmed with sequence analysis.

Expression of scTCR Fusion Proteins in Mammalian Cells

The scTCR/GM-CSF and scTCR/IFNα fusion protein constructs have been stably transfected into CHO-K1 cells. Stable transfectants secreting TCR fusion proteins were selected using ELISA assays with antibodies specific to either the TCR or cytokine portions of the fusion proteins. The highest expressing clones were expanded for static culture production of large quantities of the fusion proteins, which have been purified from cell supernates by immunoaffinity chromatography using an anti TCR Cβ monoclonal antibody. Purified proteins have been evaluated by SDS-PAGE and Coomassie staining, and each migrates at the appropriate apparent molecular weight under both reducing and non-reducing conditions. Taken together, these analyses indicate that the transfected CHO cells produce fusion proteins of the expected molecular mass and that they are properly folded, assembled, and secreted.

MHC/Peptide Binding of the TCR Portion of the 264scTCR/GM-CSF Fusion Protein

The ability of the 264scTCR/GM-CSF fusion protein to bind to peptide loaded MHC was determined by flow cytometry. T2 lymphoblast cells were loaded with p53 (aa 264-272) or p53 (aa 149-157) (control) peptide and stained with 264scTCR/GM-CSF fusion protein. Staining was detected with anti-Cβ antibodies to detect the TCR portion of the fusion protein or anti-GM-CSF to detect the GM-CSF portion of the 264scTCR/GM-CSF fusion protein. Cells loaded with p53 (aa 264-272) stained positively with 264scTCR/GM-CSF when detected with both the anti-Cβ and anti-GM-CSF antibodies (FIG. 30—pink line). T2 cells loaded with p53 (aa 149-157) did not stain with 264scTCR/GM-CSF when detected with anti-GM-CSF detection antibody. The lack of staining by 264scTCR/GM-CSF when T2 cells were loaded with the control peptide indicates that staining by this protein is mediated by the TCR component and that the staining is specific for the appropriate peptide. These data indicate that the TCR portion of the 264scTCR/GM-CSF fusion protein is capable of recognizing and specifically binding to MHC loaded with the appropriate peptide.

Bioactivity of the Human GM-CSF Portion of the 264scTCR/GM-CSF Fusion Protein

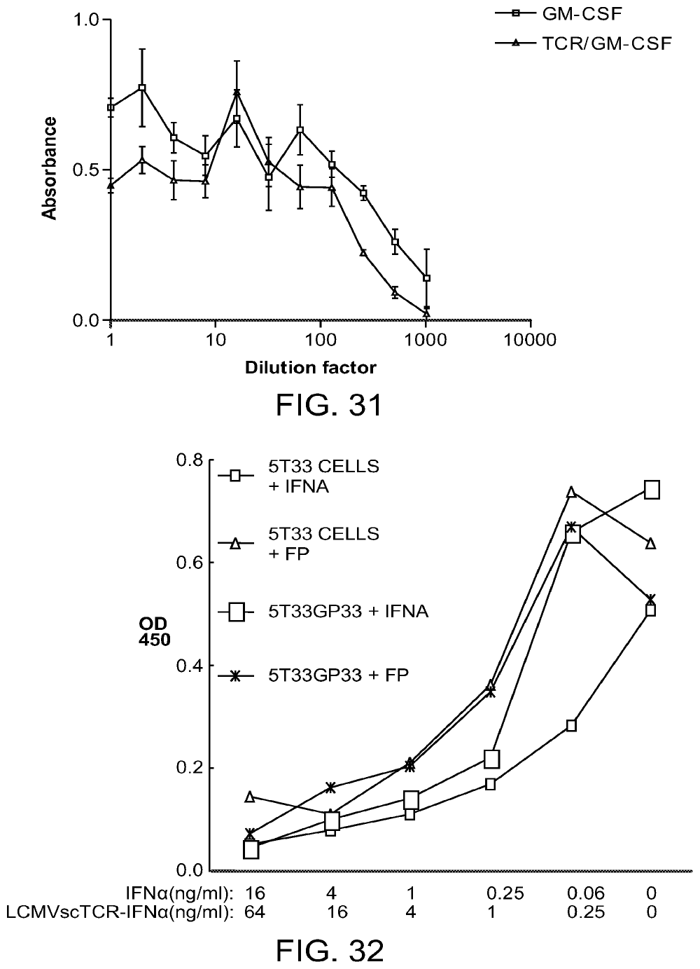

To demonstrate biological activity of the GM-CSF portion of the 264scTCR/GM-CSF fusion protein, GM-CSF dependent TF-1 cells were cultured with either 264scTCR/GM-CSF or recombinant GM-CSF at various concentrations for 21 hours and cell viability was assessed using WST-1 as described above. The ability of GM-CSF or the GM-CSF portion of 264scTCRGM-CSF to support growth of the TF-1 cells was dose dependent, wherein there was more cell proliferation at higher doses of either recombinant GM-CSF or 264scTCR/GM-CSF (FIG. 31). Further, there were similar levels of cell proliferation when equivalent molar amounts of either recombinant GM-CSF or 264scTCR/GM-CSF were used, indicating that the GM-CSF portion of 264scTCR/GM-CSF has similar activity as recombinant GM-CSF.

### Biological Activity of LCMVscTCR/IFNα Fusion Construct

The LCMVscTCR/IFNα was shown to bind the cognate peptide/MHC complex and was tested for IFNα activity in an WST1-based assay to measure its effects on the growth of murine myeloma cell lines. As shown in FIG. 32, the LCMVscTCR-IFNα fusion protein (FP) had equal or better biological activity as the equivalent molar levels of IFNα in inhibiting the growth of 5T33 or 5T33GP33 myeloma cells as assessed by the WST-1 proliferation assay described above.

Together the studies described in these example demonstrate that the cytokine and growth factor domains of the scTCR fusion proteins retain full biological activity. The scTCR domains also retain the ability to bind the cognate peptide/MHC complexes.

Although a preferred embodiment of the invention has been described using specific terms, such description is for illustrative purposes only, and it is to be understood that changes and variations may be made without departing from the spirit or scope of the following claims.

### Example 30

### Phase 1 Clinical Trial of ALT-801

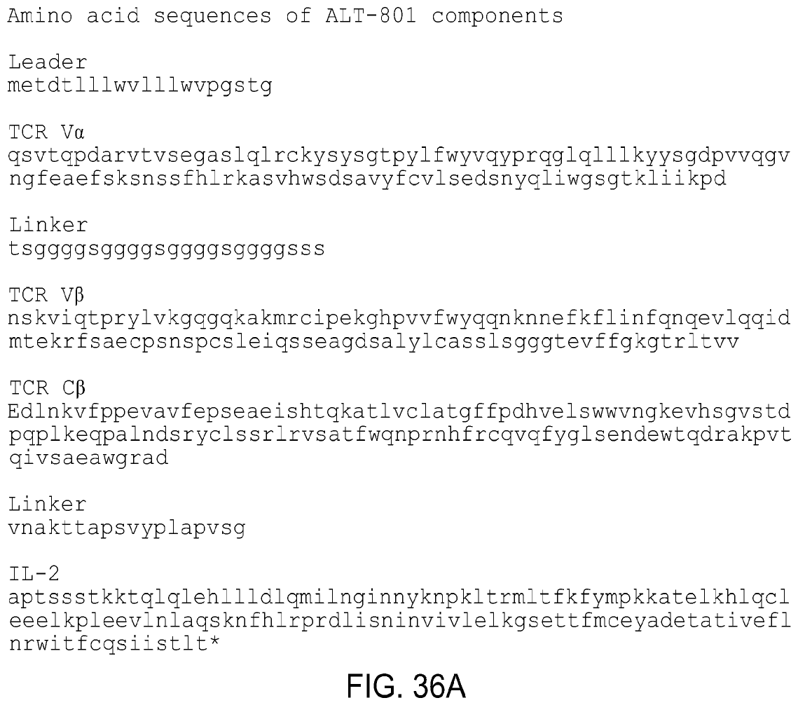

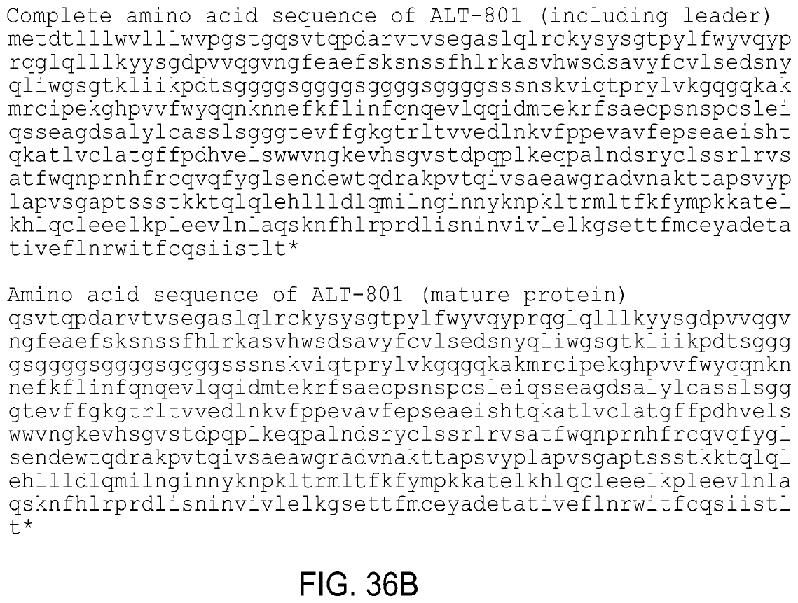

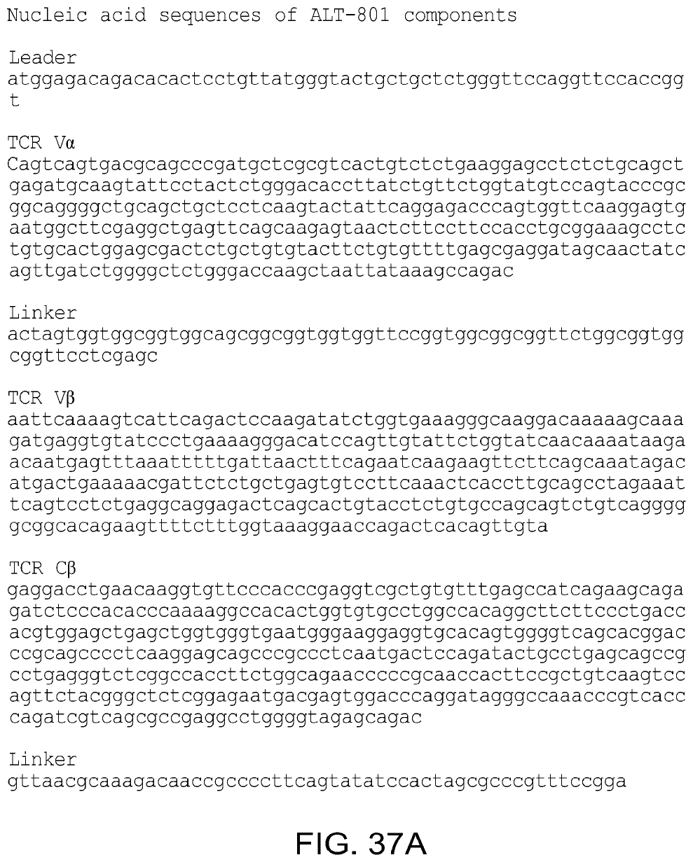

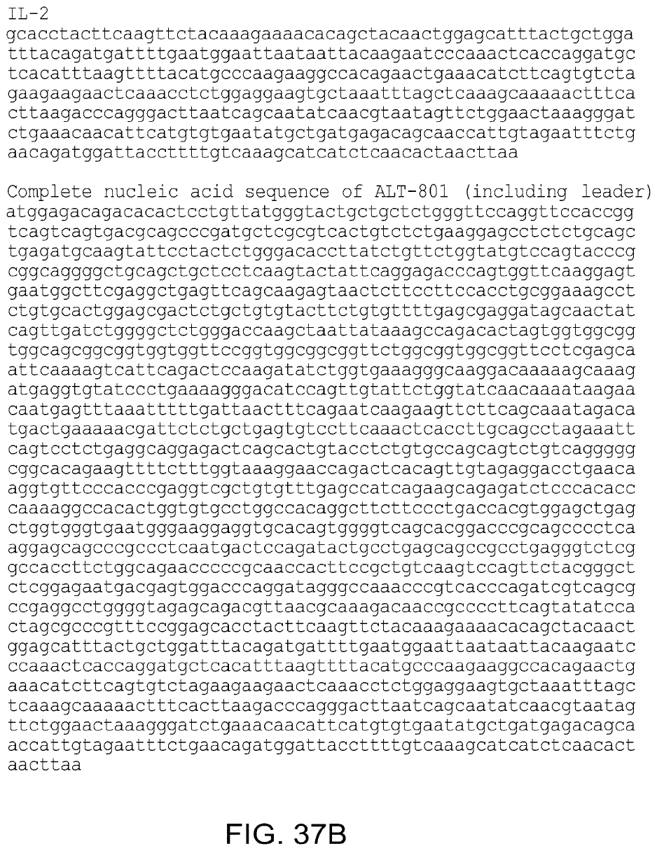

ALT-801 (c264scTCR-IL2) is a recombinant humanized soluble single-chain TCR-cytokine fusion protein, consisting of 50% human, 45% murine and 5% artificial linker amino acid sequences. The molecule is composed of human TCR β chain constant domain and murine TCR antigen-binding regions and human cytokine IL2. The chimeric T-cell receptor domain is specific for a human p53 antigen-derived peptide (aa 264-272) presented in the context of HLA-A2.1. The amino acid sequences and nucleic acid sequence of ALT-801 and its component domains are shown in FIGS. 36 and 37.

The calculated molecular weight of ACT-801 based on the amino acid sequence of the mature protein is 59,224 daltons (Apparent Molecular Weight of ˜62,000 daltons). ALT-801 is produced by a cell line that was created by transfecting a CHO-K1 cell line with the plasmid carrying the gene of the fusion protein. ALT-801 is formulated for delivery in a final composition of 2 mg/mL ALT-801 in PBS (Formulation: 1.36 g/L KH₂PO₄, 2.68 g/L Na₂HPO₄.7H₂O, 8.18 g/L NaCl pH 7.4).

ALT-801 is currently being evaluated in a multi-course dose-escalation Phase I/IIa clinical study in patients with metastatic malignancies that express HLA-A2/p53 peptide complexes, the antigenic target of ALT-801. The primary endpoints in this dose-escalation study are to evaluate the safety, determine the maximum-tolerated dose (MTD), and characterize the pharmacokinetic profile of ALT-801 administered to patients with progressive metastatic malignancies. Secondary endpoints include evaluation of ALT-801 immunogenicity and assessment of the patient's immune response and anti-tumor response. Each subject enrolled receives up to a total of 8 doses of the study drug in the following schedule: once daily as an intravenous bolus for the first 4 days (cycle 1), followed by a 10-day rest period of no study drug treatment (rest period) and then once daily as an intravenous bolus for an additional 4 days (cycle 2). The trial design includes a dose escalation phase of 3-6 subjects/cohort with starting dose of 0.015 mg/kg (equivalent to approximately 50,000 IL-2 IU/kg) and an expansion phase with 10 additional patients treated at the MTD level. At least 23 patients have been treated with at least one dose of ALT-801 at the 0.015 mg/kg (4 patients), 0.04 mg/kg (13 patients) and 0.08 mg/kg (6 patients).

The safety profile of ALT-801 in human subjects shows typical IL-2 effects though much less severe than those reported for intravenous high-dose rhIL-2 treatment. Most of the subjects who received ALT-801 at 0.015, 0.04 or 0.08 mg/kg developed fever, rigors, rash, or chills. These expected adverse events, also typically associated with uses of cytokines, were rated as mild or moderate and are controllable. Patients also experienced asymptomatic electrolyte imbalance rated as mild to severe. Table 1 provides a direct comparison of the common adverse events previously reported for high-dose rhIL-2 in patients with metastatic melanoma and renal cell carcinoma with those events observed with ALT-801 treatment at the MTD level in 10 patients. These data demonstrate that ALT-801 treatment cause significantly less hypotension and renal and liver impairment associated with capillary leak syndrome reported for high-dose IL-2-therapy. Immune response of the subjects to ALT-801 was also assessed and showed an increase in anti-ALT-801 antibody titers 4 weeks post-treatment that decreased by week 11 with no IL-2 neutralizing activity detectable at any time point. These results are comparable to those reported for patients treated with high-dose rhIL-2 (Proleukin® Drug Label).

Pharmacokinetic analysis of serum level ALT-801 in treated patients indicated that the maximum concentration achieved 15-30 minutes after drug infusion was equivalent to that expected based on the dose administered. In contrast, it has been reported that only ˜30% of the administered IL-2 dose is detectable in patients' serum in high-dose IL-2 therapy. The serum half-life of ALT-801 was approximately 3.3 hrs in patients of each of these cohorts, indicating that the fusion protein has ˜15-fold longer half life than that reported for IL-2. The ALT-801 molecule appears to be intact throughout the study, based on the results of two ELISA-based assays. These data support administration of ALT-801 as a once-daily infusion rather than the every 8 hour infusion required for high-dose IL-2 treatment. One of the major limitations with high dose IL-2 therapy is the dosing schedule and requirement for specialized ICU-level in-patient care equivalent to manage systemic side effects during drug administration. The once daily dosing schedule and reduced toxicity of ALT-801 will allow it to be administered on an out-patient basis that greatly reduces the patient care needs and cost of high dose IL-2 treatment.

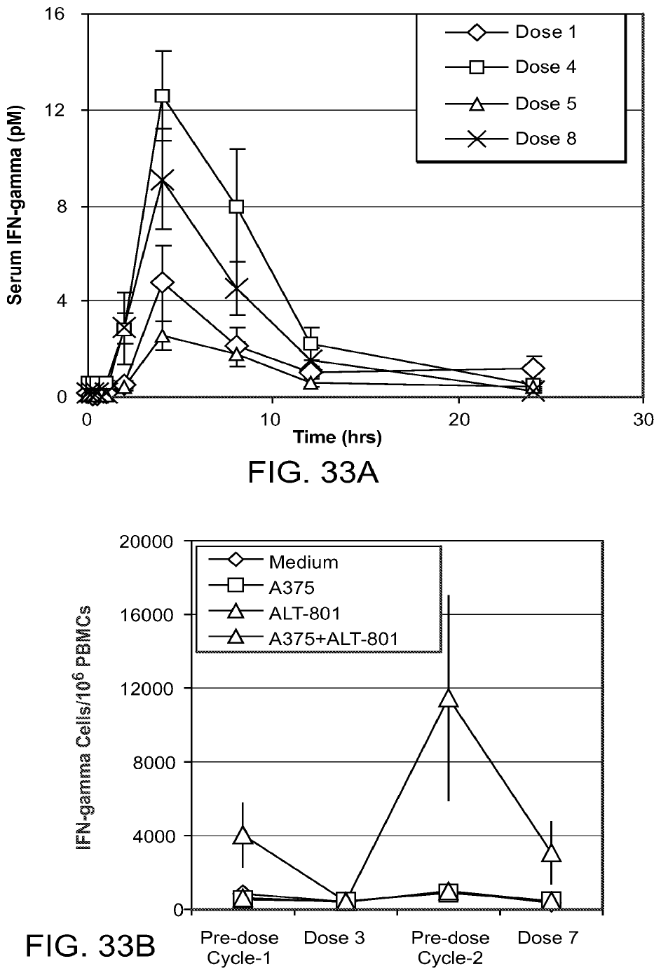

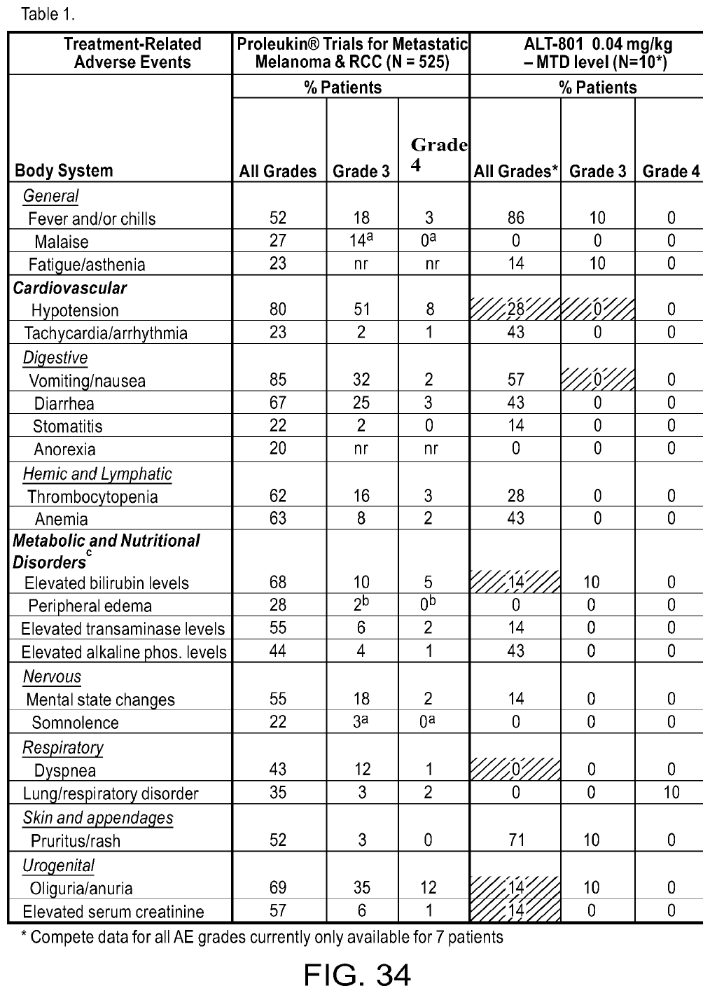

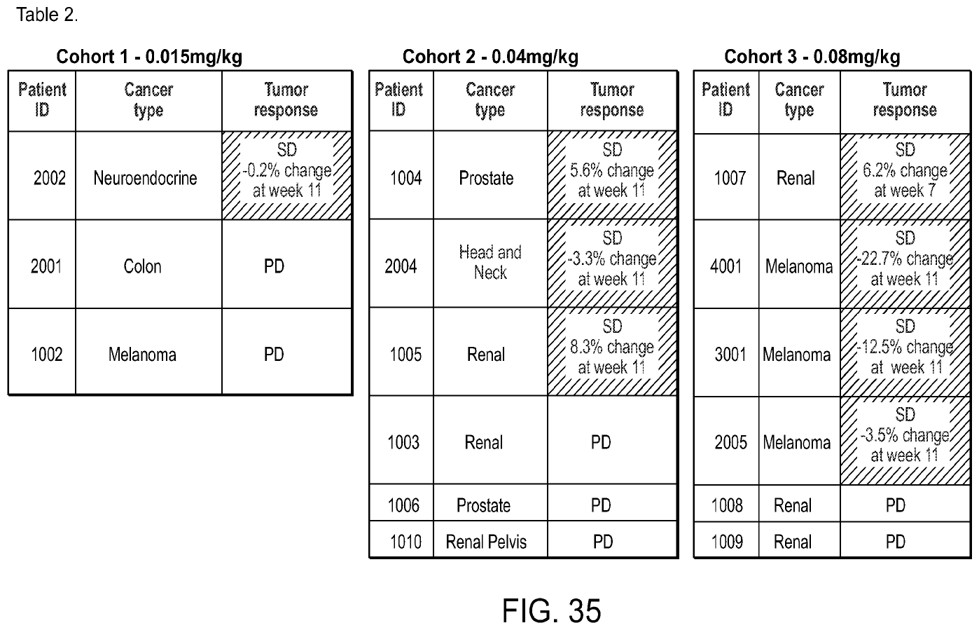

Based on PK profile, the IL-2 receptors on the surface of immune cells (i.e. NK cells and T-cells) in the patient are anticipated to be fully occupied by the ALT-801 circulating in the patient's serum when a dose of 0.015 mg/kg or higher is administered. Consistent with this assumption, an increase in serum IFN-γ concentrations was seen 4-8 hours after dosing, particularly following the fourth dose in a majority of subjects (FIG. 33A). Additionally, patients treated with 0.04 mg/kg or 0.08 mg/kg ALT-801 showed equivalent levels of serum IFN-γ induction that were higher than those observed at the 0.015 mg/kg dose level, suggesting a plateau of immune cell stimulation at the 0.04 mg/kg dose. IFN-γ is produced by activated NK and T-cells and its induction by ALT-801 likely correlates with an increase in effector cell cytolytic activity that plays a role in the antitumor responses. In contrast, little to no serum TNF-α was detected in any of the treated subjects' blood samples. TNFα is known to be induced in patients' sera following rhIL-2 treatment. The induction of TNF is also considered as one of the possible casual factors for rhIL-2 toxicity in humans. Thus the lack of treatment-related TNF-α expression may contribute to the reduced toxicity observed with ALT-801 treatment compared with high-dose IL-2.

The effects of ALT-801 on cell-mediated immune responses were also examined in treated patients. Blood samples were collected from subjects at pre-dosing on the first study drug infusion day and 2 hours after the drug infusion on the third drug infusion day during the first and second treatment cycles. Peripheral blood mononuclear cells (PBMCs) were prepared and incubated overnight in medium alone or medium containing ALT-801, p53-positive A375 tumor cells or ALT-801+A375 tumor cells. Levels of activated immune effector cells were measured using ELISPOT for interferon-gamma (IFN-γ) production. Overall, little or no increase in IFN-γ-positive cells in the subject's blood was observed during the 2-cycle course of ALT-801 treatment. However, an increase in immune cells responsive to ALT-801+A375 tumor cells was seen following completion of the first dosing cycle (Pre-dose Cycle-2 sample), suggesting that ALT-801 treatment results in stimulation and/or proliferation of tumor reactive immune cells (FIG. 33B). These cells were not found in the blood during the treatment cycle (i.e. Dose 3 and Dose 7 samples), consistent with lymphocyte migration to the tumor site. A similar pattern of stimulation was observed in subjects of each cohort with increased levels seen at higher dose levels. These results are consistent with in vitro studies demonstrating that ALT-801 at the concentrations observed in patients was able to fully activate human PBMCs and purified NK cells in vitro following overnight incubation. Together, these findings indicate that ALT-801 treatment at the MTD level provided favorable pharmacokinetics and immunostimulatory activity necessary for antitumor responses.

The effects of ALT-801 on tumors was assessed based on RECIST criteria by comparing baseline (pre-dose) CT scans with those taken in follow-up visits at week 7 and 11. As shown in Table 2, one patient with a metastatic neuroendocrine tumor achieved stable disease status after receiving a full course of ALT-801 at the 0.015 mg/kg level. Additionally, a second cohort 1 patient (renal cell carcinoma) who received only a single 0.015 mg/kg infusion was found to have stable disease based on CT scans performed both three weeks and seven weeks after dosing (data not shown). Of the six subjects in cohort 2, three (prostate cancer, head and neck cancer and renal cell carcinoma) have achieved stable disease status. Similarly, four (three with melanoma and one with renal cell carcinoma) of six evaluated subjects of cohort 3 showed stable disease with tumor shrinkage up to 23%. Thus, approximately 50% of the patients treated to date have achieved stable disease status 11 weeks after treatment. As indicated above, one subject (02004 Metastatic Head and Neck) has completed a second course of ALT-801 treatment (0.04 mg/kg) and continues to exhibit stable disease (−3.3% change in target lesion LD compared to baseline) over 9 month since initiation of ALT-801 treatment.

1-91. (canceled)

92. A soluble T cell receptor fusion molecule, the molecule comprising an amino acid sequence at least 97% identical to SEQ ID NO:16.

93. The molecule according to claim 92, wherein the amino acid sequence has at least 98% identity to SEQ ID NO:16.

94. The molecule according to claim 93, wherein the amino acid sequence has at least 99% identity to SEQ ID NO:16.

95. The molecule according to claim 94, wherein the amino acid sequence has 100% identity to SEQ ID NO:16.

96. A nucleic acid sequence encoding the molecule of claim 1.

97. The nucleic acid sequence of claim 96, wherein the nucleic acid sequence is set forth in SEQ ID NO:24.

98. A therapeutic composition, comprising a therapeutically effective amount of the molecule of claim 92 and a sterile, pharmaceutically acceptable carrier vehicle.

99. A method of treating a disorder comprising administering to a mammal a therapeutically effective amount of the composition of claim 98.

100. The method of claim 99, wherein treatment with the composition provides better efficacy than equivalent treatment with the molecule alone.

101. The method of claim 99, wherein treatment with the composition provides less toxicity than equivalent treatment with the molecule alone.

102. A heterotrimeric protein complex, the complex comprising:
a) the molecule of claim 92;
b) a human interleukin 2 (IL-2) receptor; and
c) p53.

103. The complex of claim 102, wherein the IL-2 receptor is present on the surface of a human peripheral blood mononuclear cell.

104. The complex of claim 103, wherein the p53 is present on the surface of a human tumor cell.

105. The complex of claim 104, wherein the tumor cell is a chronic myelogenous leukemia (CML) cell.
